(19) United States
(12) Patent Application Publication
(10) Pub. No.: US 2024/0336571 A1
(43) Pub. Date: Oct. 10, 2024

(54) NOVEL CYCLOPROPYL COMPOUNDS

(71) Applicants: ICAGEN, LLC, Durham, NC (US); HOFFMANN-LA ROCHE INC., Little Falls, NJ (US)

(72) Inventors: Mark CHAPMAN, Durham, NC (US); Nicholas Gareth Morse DAVIES, Wokingham (GB); Aaron GERLACH, Durham, NC (US); Christopher John GRAHAM, Durham, NC (US); Sylvain LEBRETON, Durham, NC (US); Ronghua LI, Tucson, AZ (US); Nina MA, Durham, NC (US); Daniel MADDOX, Durham, NC (US); Ingrid MECHIN, Durham, NC (US); David MOWREY, Durham, NC (US); Karthigeyan NAGARAJAN, Durham, NC (US); Anil NAIR, Durham, NC (US); Roger David NORCROSS, Aargau (CH); Roger Lluis REDONDO PENA, Basel (CH); Alena SAFAROVA, Durham, NC (US); Martin SMRCINA, Durham, NC (US)

(21) Appl. No.: 18/749,828

(22) Filed: Jun. 21, 2024

Related U.S. Application Data
(63) Continuation of application No. PCT/US2022/053308, filed on Dec. 19, 2022.
(60) Provisional application No. 63/292,482, filed on Dec. 22, 2021.

(51) Int. Cl. C07D 221/00; A61K 31/4433; A61K 31/4439; C07D 239/26; C07D 401/04; C07D 405/12; C07D 413/04; C07D 417/04

(52) U.S. Cl. CPC C07D 221/00; A61K 31/4433; A61K 31/4439; C07D 239/26; C07D 401/04; C07D 405/12; C07D 413/04; C07D 417/04

(57) ABSTRACT

The invention provides new heterocyclic compounds having the general formula (I′), or a solvate or a pharmaceutically acceptable salt thereof:

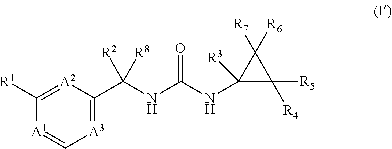

wherein R¹, R², R³, R⁴, R⁵, R⁶, R⁷ and R⁸ are as defined herein, compositions including the compounds, processes of manufacturing the compounds and methods of using the compounds.

### FIELD OF THE INVENTION

The present invention relates to novel pyridine compounds useful as Kv7.2 enhancers (or positive modulators), their manufacture, pharmaceutical compositions, kits comprising the compounds, and their use as medicaments for the therapeutic and/or prophylactic treatment of a disorder, disease, or disability associated with Kv7.2. These disorders, diseases, or disabilities can be selected from behavioral disorders, mood disorders, neurodevelopmental disorders, intellectual disability, epilepsies, neurodegenerative diseases, pain, migraine, and tinnitus.

### BACKGROUND OF THE INVENTION

The potassium channel family 7, or Q, contains five proteins that in humans are encoded by the genes KCNQ1, KCNQ2, KCNQ3, KCNQ4, and KCNQ5. The KCNQ proteins form homo- and hetero-tetrameric channels that respond to membrane voltage changes and open to let potassium ions flow out of cell membranes. Homomeric Kv7.2 channels as well as heteromeric Kv7.2 and Kv7.3 channels have been investigated because of their unique distribution and their potential role as primary regulators of neuronal excitability in many CNS and PNS pathways (Wang et al., 1998). KCNQ2 channels control the neuronal resting membrane potential, the spike frequency adaptation of neuronal firing, and presynaptic release. Impairment in their function leads to network instability even when lost exclusively in inhibitory neurons (Soh et al., 2018).

A significant percentage of childhood epilepsies are associated with KCNQ2 mutations (Lee et al., 2019). Human genetic studies identify de novo mutations in KCNQ2 as the third most robust link to epileptic encephalopathy (EE) (Zhao et al., 2020). Whether primary or secondary EEs, seizure activity worsens clinical outcomes and alters normal neurodevelopment (von Deimling, Helbig and Marsh, 2017).

Pediatric epilepsies affect about 1 in 200 children (Waaler et al., 2000) driving cognitive, behavioral and neurological deficits (Simkin and Kiskinis, 2018). In the case of specific pathogenic KCNQ2 mutations, despite most individuals eventually becoming seizure-free, developmental delays are experienced by the majority of patients (Kato et al., 2013). Targeting Kv7 channels offers a genetically validated target against epilepsy with a differentiated mode of action amongst anti-epileptics (Gunthorpe, Large and Sankar, 2012). Kv7.2 enhancers show the potential to transform neurodevelopmental trajectories by treating the neural network instability responsible for EEs (Kessi et al., 2020).

The connection between epilepsy and autism is robust (Srivastava and Sahin, 2017) and derives from the convergent phenotypes driven by a multitude of small genetic contributors, in combination with environmental factors. KCNQ2 is one of the top 5 ion channels associated with Autism Spectrum Disorder (ASD) and one of the top 30 of all de novo mutations known in ASD (Zhao et al., 2020).

Another defining feature of ASD, Atypical Sensory Processing (ASP) (Thye et al., 2018), is also driven by convergent genetics as seen in co-twin-control studies (Neufeld et al., 2021). The biology responsible for increased sensory sensitivity has been studied in preclinical models. There, multi-sensory neuronal hyper-excitability emerges regardless of the genetic manipulation that originally drives pathological neurodevelopment. Some genes whose manipulation leads to sensory sensitivity include CNTNAP2 (Peñagarikano et al., 2011), SHANK3 (Holder and Quach, 2016) and GABRB3 (Tanaka et al., 2012). Kv7.2 enhancers show the potential to correct neurodevelopmental trajectories in ASD by normalizing network stability, neural information processing, and sensory abnormalities, ultimately responsible for atypical social and repetitive behaviors in ASD. It is also interesting that KCNQ2 knock-out mice show repetitive behaviors and aberrant exploratory and social behaviors (Kim et al., 2019).

Kv7.2 enhancers also showed promise in syndromic neurodevelopmental disorders in part because of the prevalence and impact of epilepsies (Budisteanu et al., 2020). For example, epilepsy is prevalent (>80%) in Angleman syndrome, mostly starting before 3 years of age (Fiumara et al., 2010).

Another neurodevelopmental disorder, Dup15q syndrome (Dup15q), is caused by the partial duplication of Chromosome 15 that confers a considerable risk for autism spectrum disorder, epilepsy, and intellectual disability. Dup15q patient-derived induced pluripotent cells show KCNQ2 anomalies, and Retigabine, a pan-Kv7 channel opener, partially corrects their phenotype (Fink et al., 2018). Epilepsies are central to Dup15q, with Kv7.2 enhancers showing potential to transform this neurodevelopmental disorder.

In Fragile X syndrome, about 15% of individuals experience epilepsy (Berry-Kravis, 2002) together with abnormal sensory processing (McCullagh et al., 2020). KCNQ2 (Kv7.2 gene) is downregulated in the absence of Fragile X Mental Retardation Protein (FMRP) in rodent models (Zhang et al., 2018). Therefore, Kv7.2 enhancers could positively impact Fragile X by acting on both epilepsies and sensory processing.

Infantile epilepsies are associated with intellectual disabilities, and KCNQ2 de novo mutations are significantly associated with intellectual disability (Zhao et al., 2020). Kv7.2 enhancement may address the underlying biology that exacerbates the disability.

For all these neurodevelopmental disorders, early diagnosis and the identification of the correct antiepileptic treatment is at the core of the strategies aiming at normalizing neurodevelopmental trajectories.

Within behavioral disorders, Kv7.2 enhancers showed promise in attention-deficit hyperactivity disorder (ADHD) as well as major depressive disorder (MDD, depression). Some patients with KCNQ2 mutations and mild epilepsy phenotype, show cognitive delay and ADHD (Lee et al., 2019). Kv7.2 enhancers were suggested to treat the neural network instability and the behavioral impulsivity linked to ADHD. In the MDD space, Retigabine (Kv7 opener) showed antidepressant efficacy in patients by acting on the brain's reward centers (Tan et al., 2018). The significant reduction in depressive symptoms observed with retigabine places Kv7.2 enhancers as therapeutic candidates in MDD.

The therapeutic potential of Kv7.2 enhancers in pain sensitivity is supported by the localization of Kv7.2 channels in dorsal root ganglia and their established role in pain perception (Brown and Passmore, 2009). Non-selective Kv7.2 enhancers showed efficacy in reducing the excitability of human peripheral axons (Lang et al., 2008). Retigabine has already shown some efficacy in preclinical pain models (Korsgaard et al., 2005; Xu et al., 2010; Wu et al., 2017). Retigabine also shows efficacy in controlling spreading depression, a wave of cellular depolarization associated with migraines (Aiba and Noebels, 2021).

Within sensory abnormalities, aberrant plasticity of KCNQ2 channels is strongly linked to the induction of tinnitus (Li, Choi and Tzounopoulos, 2013). This link is based on the localization of Kv7.2 channels in the cochlea (Jin et al., 2009) and how cochlear damage depends on neuronal excitability driven by the closure of Kv7.2 channels (Liu, Glowatzki and Fuchs, 2015). Retigabine prevents the development of tinnitus in preclinical models (Li, Choi and Tzounopoulos, 2013). As evidence in support of how KCNQ2 pathologies are connected between indications, it is interesting to find that tinnitus and hyperacusis are more prevalent in ASD than in the general population (Danesh et al., 2015).

In neurodegenerative diseases, dysregulated K+ homeostasis in chronic neuro-inflammatory conditions is central to disease progression. For example, in amyotrophic lateral sclerosis (ALS), a fatal neurodegenerative disease of the motor nervous system (Hardiman et al., 2017), diverse genetics converge onto motorneuron excitotoxicity (Kanai et al., 2006; Pasinelli and Brown, 2006) and specifically axonal hyperexcitability predicts survival (Kanai et al., 2012). Patient-derived motor neurons show membrane hyperexcitability and the tool compound Retigabine (pan-Kv7 enhancer) rescues phenotype (Wainger et al., 2014). The motorneuron hyperexcitability was found early in presymptomatic in vivo systems (Kuo et al., 2004) where it is a contributor to disease progression. Recently, clinical trials in ALS with Retigabine showed efficacy on functional biomarkers of ALS (Wainger et al., 2021) and preclinically protects against peripheral neuropathy (Nodera et al., 2011).

In Alzheimer's disease (AD), neuronal hyperexcitability and network instability (Frere and Slutsky, 2018) are early features of both IPSC models of sporadic AD (Ghatak et al., 2019), and genetic in vivo models (Palop et al., 2007; Kazim et al., 2017; Styr and Slutsky, 2018). Network instability worsens proteinopathy (Dolev et al., 2013; Frere and Slutsky, 2018) with consequences for patients (Vossel et al., 2013; Lam et al., 2017).

Because motor neuron and cortical neuron degeneration can be meaningfully slowed down by reducing aberrant neuronal activity, Kv7.2 enhancement could be an effective way to stop such aberrant activity, changing the neurodegenerative trajectory of the disease.

Therefore, enhancing the activity of Kv7.2 is a promising strategy for the treatment or prevention of diseases associated with Kv7.2. These include neurodevelopmental disorders like autism and Fragile X, epilepsy, intellectual disability, depression, attention deficit hyperactivity disorder, motor neuron excitability, pain, migraine, and sensory processing disorders.

WO2020/163268 relates to pyridine urea derivatives as KCNQ potentiators.

U.S. Pat. No. 5,384,330 relates to pharmacologically active 1,2,4-triaminobenzene derivatives modulating potassium ion channels Kv7.2-Kv7.5 (KCNQ2-KCNQ5) for the treatment of drug-resistant epilepsy. The compounds showed tolerability issues and other side effects.

To date, no agents acting on Kv7.2 are approved for the treatment of any of the diseases, disorders, or disabilities described herein, and thus there remains a need for modulators of Kv7.2 which provide a therapeutic benefit. Further, it would be beneficial to have modulators of Kv7.2 which are highly selective over other Kv7 channels. There is a need for Kv7.2 modulators which provide for a combination of favorable pharmacological properties, such as for example potency, selectivity, and metabolic stability.

It is, therefore, an object of this invention to provide selective Kv7.2 enhancers with favorable pharmacological properties useful as Kv7.2 enhancers (or positive modulators) for the therapeutic and/or prophylactic treatment of disorders, diseases, or disabilities associated with Kv7.2.

### REFERENCES

Aiba, I. and Noebels, J. L. (2021) “Kcnq2/Kv7.2 controls the threshold and bihemispheric symmetry of cortical spreading depolarization,” Brain: a journal of neurology. doi: 10.1093/brain/awab141.
Berry-Kravis, E. (2002) “Epilepsy in fragile X syndrome,” Developmental medicine and child neurology. Wiley Online Library, 44(11), pp. 724-728.
Brown, D. A. and Passmore, G. M. (2009) “Neural KCNQ (Kv7) channels,” British journal of pharmacology, 156(8), pp. 1185-1195.
Budisteanu, M. et al. (2020) “Treatment of Epilepsy Associated with Common Chromosomal Developmental Diseases,” Open life sciences. Walter de Gruyter GmbH, 15(1), pp. 21-29.
Danesh, A. A. et al. (2015) “Tinnitus and hyperacusis in autism spectrum disorders with emphasis on high functioning individuals diagnosed with Asperger's Syndrome,” International journal of pediatric otorhinolaryngology, 79(10), pp. 1683-1688.
von Deimling, M., Helbig, I. and Marsh, E. D. (2017) “Epileptic Encephalopathies-Clinical Syndromes and Pathophysiological Concepts,” Current neurology and neuroscience reports, 17(2), p. 10.
Dolev, I. et al. (2013) “Spike bursts increase amyloid-β 40/42 ratio by inducing a presenilin-1 conformational change,” Nature neuroscience, 16(5), pp. 587-595.
Fink, J. J. et al. (2018) “Hyperexcitable phenotypes in iPSC-derived neurons from patients with 15q11-q13 duplication syndrome, a genetic form of autism,” BioRxiv. biorxiv.org. Available at: https://www.biorxiv.org/content/10.1101/286336v1.abstract.
Fiumara, A. et al. (2010) “Epilepsy in patients with Angelman syndrome,” Italian Journal of Pediatrics, p. 31. doi: 10.1186/1824-7288-36-31.
Frere, S. and Slutsky, I. (2018) “Alzheimer's Disease: From Firing Instability to Homeostasis Network Collapse,” Neuron, 97(1), pp. 32-58.
Ghatak, S. et al. (2019) “Mechanisms of hyperexcitability in Alzheimer's disease hiPSC-derived neurons and cerebral organoids vs isogenic controls,” eLife, 8. doi: 10.7554/eLife.50333.
Gunthorpe, M. J., Large, C. H. and Sankar, R. (2012) “The mechanism of action of retigabine (ezogabine), a first-in-class K+ channel opener for the treatment of epilepsy,” Epilepsia, 53(3), pp. 412-424.
Hardiman, O. et al. (2017) “Amyotrophic lateral sclerosis,” Nature reviews. Disease primers, 3, p. 17071.
Holder, J. L., Jr and Quach, M. M. (2016) “The spectrum of epilepsy and electroencephalographic abnormalities due to SHANK3 loss-of-function mutations,” Epilepsia, 57(10), pp. 1651-1659.
Jin, Z. et al. (2009) “Expression and localization of K channels KCNQ2 and KCNQ3 in the mammalian cochlea,” Audiology & neuro-otology, 14(2), pp. 98-105.
Kanai, K. et al. (2006) “Altered axonal excitability properties in amyotrophic lateral sclerosis: impaired potassium channel function related to disease stage,” Brain: a journal of neurology, 129(Pt 4), pp. 953-962.
Kanai, K. et al. (2012) “Motor axonal excitability properties are strong predictors for survival in amyotrophic lateral sclerosis,” Journal of neurology, neurosurgery, and psychiatry, 83(7), pp. 734-738.
Kato, M. et al. (2013) “Clinical spectrum of early onset epileptic encephalopathies caused by KCNQ2 mutation,” Epilepsia, 54(7), pp. 1282-1287.
Kazim, S. F. et al. (2017) “Early-Onset Network Hyperexcitability in Presymptomatic Alzheimer's Disease Transgenic Mice Is Suppressed by Passive Immunization with Anti-Human APP/Aβ Antibody and by mGluR5 Blockade,” Frontiers in aging neuroscience, 9, p. 71.
Kessi, M. et al. (2020) “Intellectual Disability and Potassium Channelopathies: A Systematic Review,” Frontiers in genetics, 11, p. 614.
Kim, E. C. et al. (2019) “Heterozygous loss of epilepsy gene KCNQ2 alters social, repetitive, and exploratory behaviors,” Genes, Brain and Behavior. doi: 10.1111/gbb.12599.
Korsgaard, M. P. G. et al. (2005) “Anxiolytic effects of Maxipost (BMS-204352) and retigabine via activation of neuronal Kv7 channels,” The Journal of pharmacology and experimental therapeutics, 314(1), pp. 282-292.
Kuo, J. J. et al. (2004) “Hyperexcitability of cultured spinal motoneurons from presymptomatic ALS mice,” Journal of neurophysiology. physiology.org, 91(1), pp. 571-575.
Lam, A. D. et al. (2017) “Silent hippocampal seizures and spikes identified by foramen ovale electrodes in Alzheimer's disease,” Nature medicine, 23(6), pp. 678-680.
Lang, P. M. et al. (2008) “Retigabine reduces the excitability of unmyelinated peripheral human axons,” Neuropharmacology, 54(8), pp. 1271-1278.
Lee, I.-C. et al. (2019) “KCNQ2 mutations in childhood nonlesional epilepsy: Variable phenotypes and a novel mutation in a case series,” Molecular genetics & genomic medicine, 7(7), p. e00816.
Li, S., Choi, V. and Tzounopoulos, T. (2013) “Pathogenic plasticity of Kv7.2/3 channel activity is essential for the induction of tinnitus,” Proceedings of the National Academy of Sciences of the United States of America. National Academy of Sciences, 110(24), pp. 9980-9985.
Liu, C., Glowatzki, E. and Fuchs, P. A. (2015) “Unmyelinated type II afferent neurons report cochlear damage,” Proceedings of the National Academy of Sciences of the United States of America, 112(47), pp. 14723-14727.
McCullagh, E. A. et al. (2020) “Mechanisms underlying auditory processing deficits in Fragile X syndrome,” FASEB journal: official publication of the Federation of American Societies for Experimental Biology. doi: 10.1096/fj.201902435R.
Neufeld, J. et al. (2021) “A co-twin-control study of altered sensory processing in autism,” Autism: the international journal of research and practice, 25(5), pp. 1422-1432.
Ng, F. L et al. (2011). Expression and function of the K+ channel KCNQ genes in human arteries. British Journal of Pharmacology, 162(1), 42-53.
Nodera, H. et al. (2011) “Neuroprotective effects of Kv7 channel agonist, retigabine, for cisplatin-induced peripheral neuropathy,” Neuroscience letters, 505(3), pp. 223-227.
Palop, J. J. et al. (2007) “Aberrant excitatory neuronal activity and compensatory remodeling of inhibitory hippocampal circuits in mouse models of Alzheimer's disease,” Neuron, 55(5), pp. 697-711.
Pasinelli, P. and Brown, R. H. (2006) “Molecular biology of amyotrophic lateral sclerosis: insights from genetics,” Nature reviews. Neuroscience, 7(9), pp. 710-723.
Peñagarikano, O. et al. (2011) “Absence of CNTNAP2 leads to epilepsy, neuronal migration abnormalities, and core autism-related deficits,” Cell, 147(1), pp. 235-246.
Simkin, D. and Kiskinis, E. (2018) “Modeling Pediatric Epilepsy Through iPSC-Based Technologies,” Epilepsy currents/American Epilepsy Society, 18(4), pp. 240-245.
Soh, H. et al. (2018) “Deletion of KCNQ2/3 potassium channels from PV+ interneurons leads to homeostatic potentiation of excitatory transmission,” eLife, 7. doi: 10.7554/eLife.38617.
Srivastava, S. and Sahin, M. (2017) “Autism spectrum disorder and epileptic encephalopathy: common causes, many questions,” Journal of neurodevelopmental disorders, 9, p. 23.
Styr, B. and Slutsky, I. (2018) “Imbalance between firing homeostasis and synaptic plasticity drives early-phase Alzheimer's disease,” Nature neuroscience, 21(4), pp. 463-473.
Tan, A. et al. (2018) “Effects of the KCNQ channel opener ezogabine on functional connectivity of the ventral striatum and clinical symptoms in patients with major depressive disorder,” Molecular psychiatry. doi: 10.1038/s41380-018-0283-2.
Tanaka, M. et al. (2012) “GABRB3, Epilepsy, and Neurodevelopment,” in Noebels, J. L. et al. (eds.) Jasper's Basic Mechanisms of the Epilepsies. Bethesda (MD): National Center for Biotechnology Information (US).
Thye, M. D. et al. (2018) “The impact of atypical sensory processing on social impairments in autism spectrum disorder,” Developmental cognitive neuroscience. Elsevier, 29, pp. 151-167.
Vossel, K. A. et al. (2013) “Seizures and epileptiform activity in the early stages of Alzheimer disease,” JAMA neurology, 70(9), pp. 1158-1166.
Waaler, P. E. et al. (2000) “Prevalence, classification, and severity of epilepsy in children in western Norway,” Epilepsia, 41(7), pp. 802-810.
Wainger, B. J. et al. (2014) “Intrinsic membrane hyperexcitability of amyotrophic lateral sclerosis patient-derived motor neurons,” Cell reports, 7(1), pp. 1-11.
Wainger, B. J. et al. (2021) “Effect of Ezogabine on Cortical and Spinal Motor Neuron Excitability in Amyotrophic Lateral Sclerosis: A Randomized Clinical Trial,” JAMA neurology, 78(2), pp. 186-196.
Wang, H. S. et al. (1998) “KCNQ2 and KCNQ3 potassium channel subunits: molecular correlates of the M-channel,” Science. American Association for the Advancement of Science, 282(5395), pp. 1890-1893.
Wu, Z. et al. (2017) “Activation of KCNQ Channels Suppresses Spontaneous Activity in Dorsal Root Ganglion Neurons and Reduces Chronic Pain after Spinal Cord Injury,” Journal of neurotrauma, 34(6), pp. 1260-1270.
Xu, W. et al. (2010) “Activation of voltage-gated KCNQ/Kv7 channels by anticonvulsant retigabine attenuates mechanical allodynia of inflammatory temporomandibular joint in rats,” Molecular pain, 6, p. 49.
Zhang, F. et al. (2018) “Fragile X mental retardation protein modulates the stability of its m6A-marked messenger RNA targets,” Human molecular genetics. Oxford Academic, 27(22), pp. 3936-3950.
Zhao, G. et al. (2020) “Gene4Denovo: an integrated database and analytic platform for de novo mutations in humans,” Nucleic acids research. Oxford Academic, 48(D1), pp. D913-D926.

### SUMMARY OF THE INVENTION

In some aspects, provided herein is a compound of formula (I′), or a solvate or a pharmaceutically acceptable salt thereof:

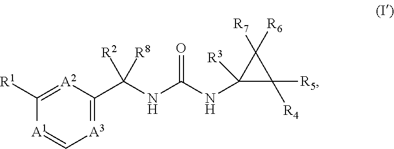

wherein R¹, R², R³, R⁴, R⁵, R⁶, R⁷, and R⁸ are as defined herein.

In a further aspect, the present invention provides a pharmaceutical composition comprising a compounds of formula (I′), or a solvate or a pharmaceutically acceptable salt thereof.

In a further aspect, the present invention provides a compound of formula (I′), or a solvate or a pharmaceutically acceptable salt thereof, or a pharmaceutical composition comprising the same, for use as therapeutically active substance.

In a further aspect, the present invention provides a compound of formula (I′), or a solvate or a pharmaceutically acceptable salt thereof, or a pharmaceutical composition comprising the same, for use in the therapeutic and/or prophylactic treatment of a disorder, disease, or disability associated with Kv7.2.

In a further aspect, the present invention provides the use of a compound of formula (I), or a solvate or a pharmaceutically acceptable salt thereof, or a pharmaceutical composition comprising the same, in the therapeutic and/or prophylactic treatment of a disorder, disease, or disability associated with Kv7.2.

In a further aspect, the present invention provides the use of a compound of formula (I′), or a solvate or a pharmaceutically acceptable salt thereof, or a pharmaceutical composition comprising the same, for the manufacture of a medicament for the therapeutic and/or prophylactic treatment of a disorder, disease, or disability associated with Kv7.2.

In a further aspect, the present invention provides a method for the therapeutic and/or prophylactic treatment of a disorder, disease, or disability associated with Kv7.2, which method comprises administering a therapeutically effective amount of a compound of formula (I′), or a solvate or a pharmaceutically acceptable salt thereof, or a pharmaceutical composition comprising the same.

In a further aspect, the present invention provides a kit for use in the therapeutic and/or prophylactic treatment of a disorder, disease, or disability associated with Kv7.2, comprising:
a) a compound of formula (I′), or a solvate or a pharmaceutically acceptable salt thereof, or a pharmaceutical composition or a pharmaceutical composition for use comprising the same; and
b) instructions for use.

The compounds of formula (I′), or a solvate or a pharmaceutically acceptable salts thereof, as described herein, provide for a combination of favorable pharmacological properties, such as for example potency, selectivity, and metabolic stability.

The compounds of formula (I′), or a solvate or a pharmaceutically acceptable salts thereof, as described herein, provide for a combination of favorable pharmacological properties, such as for example potency, selectivity, and metabolic stability. A reasonable metabolic stability is important to ensure a suitable pharmacological half life, which is best achieved with compounds that have a human liver microsomal clearance <20 μL/min/mg. Selectivity within the Kv7 family is desirable to avoid actions on tissues without therapeutic potential for the indications described in this invention. For example, actions on Kv7.4 and Kv7.5 in skeletal and smooth muscle impact the function of human arteries, where KCNQ2 expression is minimal or undetected in these tissues (Ng et al 2011).

The same specific aspects as described above also form part of the present invention for a compound of formula (I), or a solvate or a pharmaceutically acceptable salt thereof:

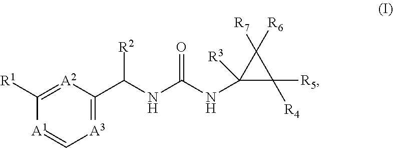

wherein R¹, R², R³, R⁴, R⁵, R⁶, and R⁷ are as defined herein.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments explicitly disclosed herein. Any embodiment described in this application can be combined with any other embodiment. The invention extends to any novel one, or any novel combination, of the features disclosed in this specification (including any accompanying claims, and abstract), or to any novel one, or any novel combination, of the any embodiment, or any steps of any method or process so disclosed.

All publications, patent applications, patents, and other references mentioned herein are incorporated by reference in their entirety.

The nomenclature used in this application is based on IUPAC systematic nomenclature, unless indicated otherwise.

Any open valency appearing on a carbon, oxygen, sulfur or nitrogen atom in the structures herein indicates the presence of a hydrogen, unless indicated otherwise. “Administer”, “administered”, or “administering” when used for the therapeutic and/or prophylactic treatment of disorders, diseases, or disabilities as described herein means the giving of a compound of this invention to a patient or subject by any method e.g. by infusion, inhalation, injection, paste, suppository, or tablet, etc.

As used herein, the terms “including”, “containing”, and “comprising” are used in their open, non-limiting sense.

The articles “a” and “an” as used in this disclosure may refer to one or more than one (e.g., to at least one) of the grammatical object of the article. By way of example, “an element” may mean one element or more than one element.

The term “substituent” denotes an atom or a group of atoms replacing a hydrogen atom on the parent molecule.

As described herein, chemical groups within the present disclosure may be “unsubstituted” or “substituted” with one or more substituents (e.g., 1, 2, 3, 4, or 5), such as those illustrated generally herein, or as exemplified by particular classes, subclasses, and species of the present disclosure. In general, the term “substituted” refers to the replacement of a hydrogen atom in a given structure with a specified substituent. In some embodiments, more than one hydrogen atom is replaced with a specified substituent (e.g. when two hydrogen atoms are replaced with one oxo substituent). Combinations of substituents envisioned by the present disclosure are typically those that result in the formation of stable or chemically feasible compounds. In one embodiment, an optionally substituted group has one substituent. In another embodiment, an optionally substituted group has two substituents. In another embodiment, an optionally substituted group has three substituents. In another embodiment, an optionally substituted group has substituents as described herein. As used herein, the term “unsubstituted” may mean that the specified group bears no substituents beyond the moiety recited (e.g., where valency is satisfied by hydrogen).

The terms “effective amount” or “therapeutically effective amount” refers to an amount of a compound of formula (I′) or (I), or a solvate or a pharmaceutically acceptable salt thereof, or a pharmaceutical composition of the afore-mentioned, being sufficient to produce a desired therapeutic outcome, such as reducing the severity of duration of, stabilizing the severity of, or elimintating one or more signs, symptoms or causes of a disease, disorder, or disability. For therapeutic use, beneficial or desired results may include, for example, decreasing one or more symptoms resulting from the disease, disorder, or disability (biochemical, histologic and/or behavioral), including its complications and intermediate pathological phenotypes presenting during development of the disease, disorder, or disability, increasing the quality of life of those suffering from the disease, disorder, or disability, decreasing the dose of other medications required to treat the disease, disorder, or disability, enhancing effect of another medication, delaying the progression of the disease, disorder, or disability and/or prolonging survival of patients.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like.

Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins, and the like.

The term “excipient” or “pharmaceutical excipients” as used herein refers to any pharmaceutically acceptable excipient that may be used in the production of a drug or pharmaceutical composition, such as a tablet containing a compound as described herein (or tautomer or pharmaceutically acceptable salt) as an active ingredient. Various substances may be embraced by the term excipient, including without limitation any substance used as a diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Disintegrant refers to excipients that expand and dissolve when wet causing the tablet to break apart in the body and release the active ingredient for absorption. Examples include cross-linked polymers like crospovidone, croscarmellose sodium, etc. and modified starches like sodium starch glycolate. Filler refers to excipients that fill out the size of a tablet by increasing the bulk volume. Fillers make it possible for the final product to have the proper volume for patient handling. Examples of fillers are plant cellulose, lactose, starch, mannitol, etc. Specific examples are lactose monohydrate like Pharmatose 200M, microcrystalline cellulose (MCC) like Avicel PH101, or Avicel PH102 and spray dried lactose like Fast Flo 316™. Binders refers to excipients that hold the ingredients in a tablet together. Binders ensure that tablets and granules can be formed with required mechanical strength. Examples of binders are, polyvinlypyrrolidon (PV), hydroxypropyl methylcellulose (HPMC), hydroxypropylcellulose (HPC), cellulose, sugar alcohols like sorbitol, proteins like gelatin and polymers like PVP, e.g. copovidone (PVP/VA 64), PEG, etc. Lubricants refer to excipients that prevent ingredients from clumping together and from sticking to the tablet punches or capsule filling machine. Lubricants also ensure that tablet formation and ejection can occur with low fraction between active ingredient and wall. Examples of lubricants are minerals like talc or silica and fats like stearin, magnesium stearate, etc. Coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch dc, sucrose, etc. Creams or lotions include, e.g., maltodextrin, carrageenans, etc. Materials for chewable tablets include, e.g. dextrose, fructose dc, lactose (monohydrate, optionally in combination with aspartame or cellulose), etc. Suspending/gelling agents include, e.g., carrageenan, sodium starch glycolate, xanthan gum, etc. Sweeteners include, e.g., aspartame, dextrose, fructose dc, sorbitol, sucrose dc, etc. Wet granulation agents include, e.g., calcium carbonate, maltodextrin, microcrystalline cellulose, etc. In some cases, the term “excipient” ecompasses pharmaceutically acceptable carriers. The skilled person knows suitable pharmaceutical compositions to be used in the treatment of patients and how to produce them.

The term a “patient” or “subject” may encompass both mammals and non-mammals. Examples of mammals may include, but are not limited to, any member of the class Mammalia: humans; nonhuman primates such as chimpanzees, monkeys, baboons, or rhesus monkeys, as well as other apes and monkey species; farm animals such as cattle, horses, sheep, goats, and swine; companion animals such as rabbits, dogs, and cats; laboratory animals including rodents, such as rats, mice and guinea pigs; and the like. Examples of non-mammals include, but are not limited to, birds, fish, and the like. “Patient” or “subject” may include both human and animals. In some preferred embodiments, the “patient” or “subject” is a human.

As used herein, the terms “treat” or “treatment” are meant to indicate a postponement of development of one or more disease(s), disorder(s), or disability(ies); preventing the development of one or more disease(s), disorder(s), or disability(ies); and/or reducing severity of one or more symptoms of a disease, disorder, or disability that will or are expected to develop. Thus, these terms may include ameliorating one or more existing disease, disorder, or disability symptoms; preventing one or more additional symptoms; ameliorating or preventing the underlying causes of one or more symptoms; inhibiting the diseases, disorder, or disability, e.g., arresting the development of the diseases, disorder, or disability; relieving the diseases, disorder, or disability; causing regression of the diseases, disorder, or disability; relieving a symptom caused by the diseases, disorder, or disability; or stopping or alleviating the symptoms of the diseases, disorder, or disability.

Compounds of the invention and disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water. In some embodiments, solvates are excluded.

The term “prophylaxis” as used herein includes: preventing or delaying the appearance of clinical symptoms of diseases, disorder, or disability developing in a patient or subject, especially a human, that may be afflicted with or predisposed to the disease, disorder, or disability as described herein, but does not yet experience or display clinical or subclinical symptoms of the disease, disorder, or disability.

As used herein, the term “about,” when referring to a value is meant to encompass variations of, for example, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller ranges is also encompassed within the invention, subject to any specifically excluded limit in the stated range. Where the stated range includes one or both of the limits, ranges excluding either or both of those included limits are also included in the invention.

Numerical ranges, as used herein, may include sequential integers. For example, a range expressed as “from 0 to 5” would include 0, 1, 2, 3, 4, and 5.

A “metabolite” is a product produced through metabolism in the body of a specified compound or salt thereof. Metabolites of a compound may be identified using routine techniques known in the art and their activities determined using tests such as those described herein. Such products may result e.g. from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

The terms “compound of this invention,” and “compounds of the present invention” “compounds of the invention”, “compounds of formula (I′) and “compounds of formula (I)” include compounds of formula (I′), compounds of formula (I), compounds selected from any of formulae (I*), (II**), (II), (III), (IV), (V), (VI), (VII), (VIII), (IX), (X), (XI) etc., compounds from Tables 1, 2, etc., stereoisomers, geometric isomers, solvates, pharmaceutically acceptable salts, tautomers, metabolites, prodrugs, polymorphs; and mixtures thereof.

The symbols “*” at the end of a bond or “- - -” drawn through a bond each refer to the point of attachment of a functional group or other chemical moiety to the rest of the molecule of which it is a part. Thus, for example:

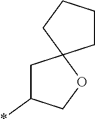 [SMILES: *C1COC2(CCCC2)C1]

means that the substituent is attached to the rest of the molecule as shown

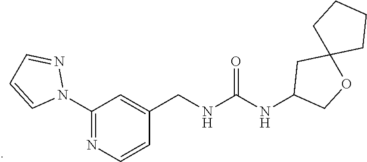 [SMILES: O=C(NCc1ccnc(-n2cccn2)c1)NC1COC2(CCCC2)C1]

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent. “Optionally substituted” means that a compound can be unsubstituted or substituted as defined herein. “The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen. In an additon, e.g. in a compound in which e.g. R² and R³ are independently selected from hydrogen and hydroxyC₁₋₆alkyl, both R² and R³ can be hydrogen, or both can be hydroxyC₁₋₆alkyl, or one of R² and R³ can be hydrogen and the other one hydroxyC₁₋₆alkyl.

In this application the units ul, uMol, C etc. mean μl, μMol, ° C. etc.

The term “EC₅₀” in this application is defined as: the agonistic effect of a compound can be determined by testing the compound in an in vitro assay as described herein, whereby the effect of the compound is measured across a range of compound concentrations. The resulting data is plotted as a concentration response curve, which typically follows a sigmoidal function, whereby the concentration of the compound is plotted on the x axis and the response (agonistic effect) is plotted on the y axis. The term “EC₅₀” is the “half maximal effective concentration” and denotes the concentration of a particular compound required to obtain 50% of the maximum response (Eₘₐₓ) which is observed for that compound in the given in vitro assay.

A compound of this invention may exist in one or more stereoisomeric forms (e.g., it contains one or more asymmetric carbon atoms). The individual stereoisomers (enantiomers and diastereomers) and mixtures of these are included within the scope of the subject matter disclosed herein.

Likewise, it is understood that a compound or salt may exist in tautomeric forms other than that shown in the formula and these are also included within the scope of the subject matter disclosed herein. It is to be understood that the subject matter disclosed herein includes combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

A compound of this invention can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “chiral” refers to molecules which have the property of non-superimposability of the mirror image partner, while the term “achiral” refers to molecules which are superimposable on their mirror image partner. Chiral separation of a racemate to its enantiomeric components may be performed to separate the eutomer and the distomer.

The term “stereoisomers” refers to compounds, which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Any organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process.

The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

It should be understood that individual enantiomers and diastereomers are included in the tables below by compound name, and their corresponding structures can be readily determined therefrom. In some instances, the enantiomers or diastereomers are identified by their respective properties, for example, retention times on a chiral HPLC or their biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

In some embodiments of the invention only one of the possible enantiomers are used. In other embodiments mixtures of the possible enantiomers having different percentages for each component are used.

In some embodiments, the compounds of this invention are isotopically-labeled by having one or more atoms therein replaced by an atom having a different atomic mass or mass number. Such isotopically-labeled (e.g., radiolabeled) compounds of formula (I′) or (I), or a solvate or a pharmaceutically acceptable salts thereof, are considered to be within the scope of this disclosure. Examples of isotopes that can be incorporated into the compounds of this inventon, include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of this invention for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of this invention can be enriched with 1, 2, 5, 10, 25, 50, 75, 90, 95, or 99 percent of a given isotope.

In some aspects, it is particularly envisaged that compounds of formula (I′) or (I), or a solvate or a pharmaceutically acceptable salts thereof, have one or more hydrogen atoms in the structure substituted for ²H (deuterium), ³H (tritium), preferably ²H (deuterium). Any of the hydrogen atoms in the structures may be substituted in this way but in some cases it is preferred that one or both of the hydrogen atoms that may be present at R² and R⁸ are substituted, particularly substituted for ²H (deuterium), ³H (tritium), preferably ²H (deuterium). In some specific aspects, R² and R⁸ are both ²H (deuterium).

Substitution with positron emitting isotopes, such as ³H, ¹¹C, ¹⁸F, ¹⁵O and ¹³N, can be useful in Positron Emission Topography (PET) studies for examining substrate receptor occupancy. Isotopically-labeled compounds of this invention can generally be prepared by conventional techniques known to those skilled in the art or by processes analogous to those described in the Examples as set out below using an appropriate isotopically-labeled reagent in place of the non-labeled reagent previously employed.

In one embodiment, the present invention provides pharmaceutically acceptable salts of the compounds of this invention especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I′) or (I), or solvates or pharmaceutically acceptable salts thereof, as described herein (i.e., as “free bases” or “free acids”, respectively).

When a bond in a compound of this invention is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

The term “alkyl” refers to a mono- or multivalent, e.g., a mono- or bivalent, linear or branched saturated hydrocarbon group, in particular a hydrocarbon group of 1 to 6 carbon atoms (“C₁₋₆alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of methyl (CH₃—), ethyl (CH₃CH₂—), n-propyl (CH₃CH₂CH₂—), 2-propyl (isopropyl, (CH₃)₂CH₂—), n-butyl (CH₃CH₂CH₂CH₂—), iso-butyl ((CH₃)₂CH₂CH₂—), sec-butyl (CH₃CH(CH₃)CH₂—), and tert-butyl ((CH₃)₃C—). Preferred, yet non-limiting, examples of alkyl are tert-butyl ((CH₃)₃C—) or methyl (CH₃—). A preferred, yet non-limiting, example of alkyl is methyl (CH₃—). Another preferred, yet non-limiting, example of alkyl is tert-butyl ((CH₃)₃C—).

The term “alkoxy” refers to an alkyl group, as previously defined, attached to the parent molecular moiety via an oxygen atom. The alkoxy group preferably contains 1 to 6 carbon atoms (“C₁₋₆alkoxy”), e.g. 1, 2, 3, 4, 5, or 6 carbon atoms. In other embodiments, the alkoxy group contains 1 to 4 carbon atoms. In still other embodiments, the alkoxy group contains 1 to 3 carbon atoms. Some non-limiting examples of alkoxy groups include CH₃O— (methoxy), CH₃CH₂O-(ethoxy), CH₃CH₂CH₂O— (n-propoxy), and (CH₃)₃CO— (tert-butoxy). A particularly preferred, yet non-limiting, example of alkoxy is methoxy (CH₃O—).

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

“Heteroaryl” as used herein refers to a 5 or 6 membered monocyclic, aromatic group comprising at least one ring heteroatom. In some embodiments, the heteroatom is independently selected from the group consisting of N-atoms, O-atoms, and S-atoms. The number of ring atoms refer to the sum of carbon and heteroatoms in the one ring. In some embodiment, heteroaryl is a 5 or 6 membered moncyclic, aromatic group with two N-atoms. In some embodiments, heteroaryl is a 5 or 6 membered monocyclic aromatic group comprising one N-atom. In some embodiments, heteroaryl is a 5 or 6 membered monocyclic aromatic group comprising one O-atom and one S-atom. In some embodiments, heteroaryl is a 5 or 6 membered monocyclic aromatic group comprising one N-atom and one O-atom. Examples of 5 membered heteroaryl groups include, but are not limited to, pyrrolyl, pyrazolyl, imidazolyl, oxazolyl, thiazolyl, triazolyl, or furanyl. In some preferred embodiments, 5 membered heteroaryl is pyrazolyl, imidazolyl, oxazolyl, or thiazolyl. Examples of 6 membered heteroaryl groups include, but are not limited to, pyrimidinyl, pyridinyl, pyrazinyl, or pyridazinyl. In some more preferred embodiments, 6 membered heteroaryl is pyrazinyl, pyridinyl or pyrimidinyl.

The term “cyano” refers to a —CN (nitrile) group.

The term “hydroxy” or “hydroxyl” refers to an OH group.

The term “haloalkyl” refers to an alkyl group wherein at least one of the hydrogen atoms of the alkyl group has been replaced by one or more halogen atoms. Preferably, “haloalkyl” refers to a C₁₋₆alkyl group wherein 1, 2 or 3 hydrogen atoms of the alkyl group have been replaced by a halogen atom, i.e. haloalkyl includes monohaloalkyl, dihaloalkyl, trihaloalkyl, perhaloalkyl and the like. Halogen atoms may be fluoro (F), chloro (Cl), or bromo (Br). Particularly preferred, yet non-limiting, examples of “halogen” within haloalkyl are fluoro (F) and chloro (Cl). More preferably, haloalkyl is substituted with fluoro (F). Preferred, yet non-limiting, examples of haloalkyl are (CH₃)₂FC— (1-fluoro-isopropyl), CF₃CH₂— (2,2,2-trifluoroethyl), CH₃CF₂— (1,1-difluoroethyl), CF₃— (trifluoromethyl), CH₂F— (fluoromethyl), or CHF₂— (difluromethyl). Particularly preferred are CH₃CF₂—, CF₃—,

“Haloalkoxy”, as used herein, refers to an alkoxy group, wherein at least one of the hydrogen atoms has been replaced by halogen atoms. Preferably, “haloalkoxy” refers to an alkoxy group, preferably a C₁₋₆alkoxy group, wherein 1, 2, or 3 hydrogen atoms of the alkyl group have been replaced by a halogen atom, i.e. haloalkoxy includes monohaloalkoxy, dihaloalkoxy, trihaloalkoxy, perhaloaloxy and the like. Halogen atoms may be fluoro (F), chloro (Cl) or bromo (Br). Particularly preferred, yet non-limiting, examples of “halogen” are fluoro (F) and chloro (Cl). More preferably, haloalkoxy is substituted with fluoro (F). Preferred, yet non-limiting, examples of haloalkoxy are CHF₂O—, CH₂FO—, CF₃CH₂O—, CF₂HCH₂O—, CH₃CF₂CH₂O—, and CH₃CFHCH₂O—. Also preferred, yet non-limiting, examples of haloalkoxy are haloC₁₋₆alkoxy selected from CHF₂O—, CH₂FO—, CF₃O—, FCH₂CFHCH₂O—, CH₂FCH₂O—, and CF₃CH₂O—. Particularly preferred are CHF₂O—, CF₃CH₂O—, CF₃O—, CH₂FO—, or FCH₂CFHCH₂O—. The haloalkoxy may be optionally substituted with C₁₋₆alkoxy, such as CF₃CH(CH₂OCH₃)O—.

The term “hydroxyalkyl” refers to an alkyl group, preferably with one to six C-atoms, wherein at least one of the hydrogen atoms of the alkyl group has been replaced by one or more hydroxy. Preferably, “hydroxyalkyl” refers to an alkyl group, preferably a C₁₋₆alkyl, wherein 1, 2 or 3 hydrogen atoms of the alkyl group have been replaced by hydroxy, i.e. hydroxyalkyl includes monohydroxyalkyl, dihydroxalkyl, trihydroxyalkyl, perhydroxyalkyl and the like. More preferably, “hydroxyalkyl” refers to an alkyl group wherein one hydrogen atom has been replaced by hydroxy. Particularly prefered, yet not limiting examples of hydroxyalkyl are HOCH₂— (hydroxymethyl), HOCH₂CH₂— (hydroxyethyl).

“4-6 membered heterocycloalkylC₀₋₆alkoxy” as used herein refers to a 4-6 membered heterocycloalkyl group which is linked to a C₀₋₆alkoxy group. C₀ means that the 4-6 membered heterocycloalkylC₀₋₆alkoxy group has the structure “4-6 membered heterocycloalkyl-O—”. In some embodiments, the heterocycloalkyl ring comprises one or more O-atom or S-atom. In some preferred embodiments, the heterocycloalkyl ring comprises one O-atom. Non-limiting examples of heterocycloalkylC₀₋₆alkoxy according to this invention are:

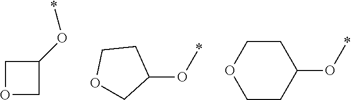

“3-6 membered cycloalkylC₀₋₆alkoxy” as used herein refers to a 3-6 membered saturated monocyclic cycloalkyl group which is linked to a C₀₋₆alkoxy group. C₀ means that the 3-6 membered cycloalkylC₀₋₆ alkoxy group has the structure “3-6 membered cycloalkyl-O—”. Non-limiting examples of heterocycloalkylC₀₋₆alkoxy according to this invention are:

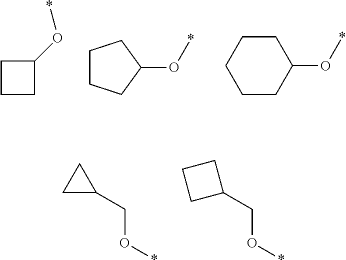

The term “saturated monocyclic 3-6 membered cycloalkyl” denotes a saturated monovalent saturated monocyclic hydrocarbon group of 3 to 6 ring carbon atoms. Examples for such monocyclic cycloalkyl are cyclopropyl, cyclobutanyl, cyclopentyl, or cyclohexyl. Preferred, but non-limiting examples are cyclopropyl, cyclobutyl, or cyclohexyl. A particularly preferred cycloalkyl is cyclohexyl. The cycloalkyl may be substituted as described herein. The cycloalkyl is preferably unsubstituted.

The term “4-6 membered heterocycloalkyl” denotes a monovalent saturated or partly unsaturated mono- or bicyclic ring system of 4 to 6 ring atoms, comprising 1, 2, or 3 ring heteroatoms selected independently from N, O and S, the remaining ring atoms being carbon. A not limiting example is oxeantyl.

“Phenoxy” as used herein refers to a phenyl group which is linked to an —O— to form an alkoxy group having the structure “phenyl-O—” optionally substituted as described herein.

The term “mood disorder” as used herein relates to a mental health problem that primarily affects a person's emotional state. It is a disorder in which a person experiences long periods of extreme happiness, extreme sadness or both. Two of the most common mood disorders are depression and bipolar disorder.

The term “depression” as used herein relates to a mood disorder that causes a persistent feeling of sadness and loss of interest. It is also known as major depressive disorder (MDD).

The term “behavioral disorder” relates to disorders that involve a pattern of disruptive behaviors in children that last for at least 6 months and cause problems in school, at home and in social situations. Behavioral disorders involve a pattern of disruptive behaviors in children that last for at least 6 months and cause problems in school, at home and in social situations. The most important behavioral disorder is Attention deficit hyperactivity disorder” (ADHD).

The term “Attention deficit hyperactivity disorder” (ADHD) as used herein relates to a behavioral disorder characterized by inattention, or excessive activity and impulsivity. ADHD occurs more frequently in people with epilepsy than in the general population. Children with ADHD have an increased risk of seizures, with approximately 14% of children with ADHD developing seizures.

The term “developmental disorder” or “neurodevelopmental disorder” as used herein relates to a group of conditions caused by an impairment in physical, learning, language, or behavior areas. These conditions begin during the developmental period, may impact day-to-day functioning, and can last through a person's lifetime. Examples of neurodevelopment disorders include Autism Spectrum Disorder (“ASD”) and syndromic developmental disorders.

The term “Autism Spectrum Disorder (ASD)” as used herein relates to a developmental disorder of variable severity that is characterized by difficulty in social interaction and communication and by restricted or repetitive patterns of thought and behavior. ASD encompasses mostly idiopathic but also syndromic forms and is currently diagnosed according to the diagnostic and statistical manual version 5 (DSM V).

The term “syndromic developmental disorder” as used herein relates to a development disorder with a clinically defined pattern of somatic abnormalities and a neurobehavioral phenotype that may include ASD. The diagnosis is typically confirmed by targeted genetic testing. Examples for syndromic development disorders include Dup15q syndrome (Dup15q), Fragile X syndrome (FXS) and Angelman syndrome.

The term “Dup15q syndrome” or “Duq15q” as used herein relates to the common name for chromosome 15q11.2-q13.1 duplication syndrome. This is a syndromic development disorder, caused by the partial duplication of Chromosome 15, which confers a strong risk for autism spectrum disorder, epilepsy and intellectual disability.

The term “Fragile X syndrome” (FXS) as used herein relates to a genetic disorder characterized by mild-to-moderate intellectual disability. This disorder is typically caused by an expansion of the CGG triplet repeat within the FMR1 (fragile X mental retardation 1) gene on the X chromosome.

The term “Angelman syndrome” as used herein relates to a genetic disorder that mainly affects the nervous system due to a lack of function of part of chromosome 15 inherited from a person's mother. Characteristic features of this condition include delayed development, intellectual disability, severe speech impairment, and problems with movement and balance (ataxia). Most affected children also have recurrent seizures (epilepsy).

The term “Intellectual disability” (ID) used herein relates to a generalized neurodevelopmental disorder characterized by significantly impaired intellectual and adaptive functioning. It is defined by an IQ under 70, in addition to deficits in two or more adaptive behaviors that affect everyday, general living. ID is also known as a general learning disability and formerly known as mental retardation (MR).

The term “epilepsy” used herein relates to a neurological disorder marked by sudden recurrent episodes of sensory disturbance, loss of consciousness, or convulsions, associated with abnormal electrical activity in the brain. Examples of epilepsies include broad pediatric epilepsies, West syndrome, Ohtahara syndrome and epileptic encephalopathy.

The term “neurodegenerative diseases” used herein relates to diseases that are related to e progressive loss of structure or function of neurons, including the death of neurons. Examples of neurodegenerative diseases include, but are not limited to, Alzheimer's disease and motor neuron diseases.

The term “motor neuron disease” used herein relates to a group of rare neurodegenerative disorders that selectively affect motor neurons. Examples of motor neuron diseases include, but are not limited to, amyotrophic lateral sclerosis (ALS).

The term “pain” as used herein relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain.

The term “migraine” as used herein relates to a moderate to severe headache disorder, causing throbbing or pulsating pain for hours or days.

The term “Tinnitus” as used herein relates to a symptom characterized by the perception of sound when no corresponding external sound is present.

Any disease, disorder, or disability described herein also includes any state or condition related to such disease, disorder, or disability.

### Compounds of the Invention

All compound names of compound structures were generated using OpenEye Lexichem, Version 1.2.0, OpenEye Scientific Software, Santa Fe, NM, USA; www.eyesopen.com.

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments described herein. Any embodiment described in this application can be combined with any other embodiment. For example, any embodiment herein relating to the compounds of this invention can be combined with any embodiment of pharmaceutical compositions, kits, medical use, or method of treatment. The invention extends to any novel one, or any novel combination, of the features disclosed in this specification (including any accompanying claims, abstract and drawings), or to any novel one, or any novel combination, of the steps of any method or process so disclosed.

In some embodiments of this invention, compounds of formula (I′) or (I) have the structures depicted from any of formulae (II′), (I*), (II**), (II), (III), (IV), (V), (VI), (VII), (VIII), (IX), (X), or (XI).

The invention provides a compound of formula (I′), or a solvate or a pharmaceutically acceptable salt thereof:

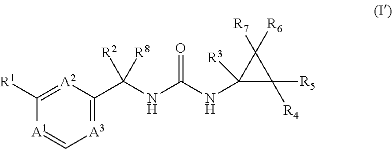

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R² is selected from H, D, and C₁₋₆alkyl;
R³ is H, C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy;
R⁷ is selected from H, C₁₋₆alkyl, haloC₁₋₆alkyl, and halogen; and
R⁸ is selected from H, D, and C₁₋₆alkyl;

In one embodiment, the invention provides a compound of formula (I), or a solvate or a pharmaceutically acceptable salt thereof:

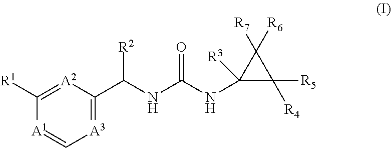

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a compound of formula (I), or a solvate or a pharmaceutically acceptable salt thereof:

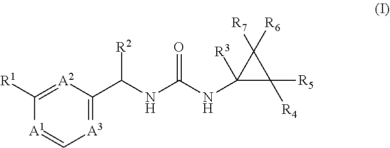

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a compound of formula (II), or a solvate or a pharmaceutically acceptable salt thereof:

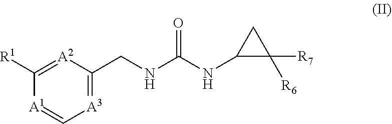

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkyl C₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a compound of formula (II′), or a solvate or a pharmaceutically acceptable salt thereof:

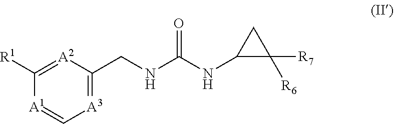

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkyl C₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

All of the features disclosed in this specification (including any accompanying claims, abstract and drawings) relating to any embodiment so disclosed, may be combined in any combination, except combinations where at least some of such features and/or steps are mutually exclusive. The invention is not restricted to the details of any embodiments described herein. Any embodiment described in this application can be combined with any other embodiment.

In one embodiment, the invention provides a compound of formula (I′), or a solvate or a pharmaceutically acceptable salt thereof:

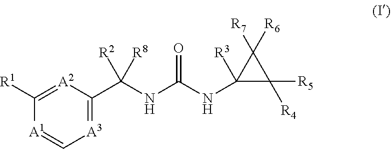

wherein
A¹ is N or CH;
A² is N or CH; and
A³ is N or CH; with the proviso that at least one of A¹, A², or A³ is N.

In one embodiment, the invention provides a compound of formula (I), or a solvate or a pharmaceutically acceptable salt thereof:

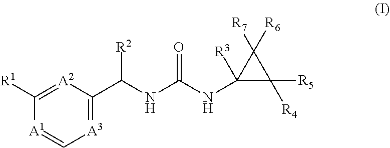

wherein
A¹ is N or CH;
A² is N or CH; and
A³ is N or CH; with the proviso that at least one of A¹, A², or A³ is N.

In this invention A¹, A², A³ in a compound of formula (I) or (I), or a solvate or a pharmaceutically acceptable salt thereof, cannot simultaneously be CH.

In one embodiment, the invention provides a compound of formula (I′) or (I), or a solvate or a pharmaceutically acceptable salt thereof, wherein one, two, or three of A¹, A², or A³ are N.

In one embodiment, the invention provides a compound of formula (I′) or (I), or a solvate or a pharmaceutically acceptable salt thereof, wherein one of A¹, A², or A³ is N. In this embodiment the other ones are CH.

In one preferred embodiment, the invention provides a compound of formula (I′) or (I), or a solvate or a pharmaceutically acceptable salt thereof, wherein only A¹ is N. In this embodiment, A² and A³ are CH.

In one embodiment, the invention provides a compound of formula (I′) or (I), or a solvate or a pharmaceutically acceptable salt thereof, wherein only A² is N. In this embodiment, A¹ and A³ are CH.

In one embodiment, the invention provides a compound of formula (I′) or (I), or a solvate or a pharmaceutically acceptable salt thereof, wherein only A³ is N. In this embodiment, A¹ and A² are CH.

In one embodiment, the invention provides a compound of formula (I′) or (I), or a solvate or a pharmaceutically acceptable salt thereof, wherein two of A¹, A², or A³ are N. In this embodiment, the remaining one is CH.

In one embodiment, the invention provides a compound of formula (I′) or (I), or a solvate or a pharmaceutically acceptable salt thereof, wherein only A¹ and A², are N. In this embodiment A³ is CH.

In one embodiment, the invention provides a compound of formula (I) or (I), or a solvate or a pharmaceutically acceptable salt thereof, wherein only A¹ and A³, are N. In this embodiment A² is CH.

In one embodiment, the invention provides a compound of formula (I′) or (I), or a solvate or a pharmaceutically acceptable salt thereof, wherein only A² and A³, are N. In this embodiment A¹ is CH.

In one embodiment, the invention provides a compound of formula (I′) or (I), or a solvate or a pharmaceutically acceptable salt thereof, wherein A¹, A², and A³ are N.

In this invention for a compound of formula (I′) or (I), or a solvate or a pharmaceutically acceptable salt thereof, at least one of A¹, A², or A³ is N and the others are independently selected from CH, CR*, CR**, and CR*** as described herein.

In some embodiments, the invention provides a compound of formula (I′) or (I), or a solvate or a pharmaceutically acceptable salt thereof, wherein R*, R**, or R*** are independently selected from halogen, haloC₁₋₆alkyl, and haloC₁₋₆alkoxy, all as described herein.

In some embodiments, the invention provides compound of formula (I′), or a solvate or a pharmaceutically acceptable salt thereof:

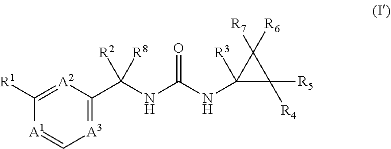

wherein
A¹ is N, CH, or CR*;
A² is N, CH; or CR**; and
A³ is N, CH, or CR***; with the proviso that at least one of A¹, A², or A³ is N,
wherein R*, R**, or R*** independently selected from halogen, haloC₁₋₆alkyl, and haloC₁₋₆alkoxy, and wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R² is selected from H, D, and C₁₋₆alkyl;
R³ H, or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy;
R⁷ is selected from H, C₁₋₆alkyl, haloC₁₋₆alkyl, and halogen; and
R⁸ is selected from H, D, and C₁₋₆alkyl

In some embodiments, the invention provides compound of formula (I), or a solvate or a pharmaceutically acceptable salt thereof:

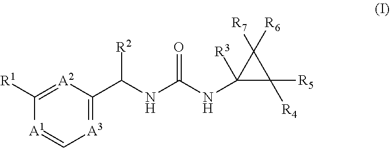

wherein
A¹ is N, CH, or CR*;
A² is N, CH; or CR**; and
A³ is N, CH, or CR***; with the proviso that at least one of A¹, A², or A³ is N,
wherein R*, R**, or R*** independently selected from halogen, haloC₁₋₆alkyl, and haloC₁₋₆ alkoxy, and wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R² is H, or C₁₋₆alkyl;
R³ is H, or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In some embodiments, the invention provides compound of formula (I), or a solvate or a pharmaceutically acceptable salt thereof:

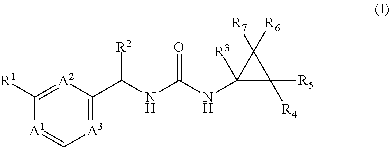

wherein
A¹ is N, CH, or CR*;
A² is N, CH; or CR**; and
A³ is N, CH, or CR***; with the proviso that at least one of A¹, A², or A³ is N,
wherein R*, R**, or R*** independently selected from halogen, haloC₁₋₆alkyl, and haloC₁₋₆alkoxy, and wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a compound of formula (I), or a solvate or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 4-6 membered cycylalkylC₀₋₆alkoxy, phenoxy, 4-6 membered heterocycloalkyl, and 4-6 membered heterocycloalkylC₀₋₆alkoxy, wherein heteroaryl, heterocycloalkyl, heterocycloalkylalkoxy, cycloalkylalkoxy, or phenoxy are optionally substituted with one, two, or three substituents independently selected from halogen, haloC₁₋₆alkyl, and haloC₁₋₆alkoxy.

In one embodiment, the invention provides a compound of formula (I), or a solvate or a pharmaceutically acceptable salt thereof, wherein R¹ is selected from cyano, halogen, 5 membered heteroaryl, C₁₋₆alkyl, C₁₋₆alkoxy, 4-6 membered cycloalkylC₀₋₆alkoxy, phenoxy, 4-6 membered heterocycloalkyl, and 4-6 membered heterocycloalkylC₀₋₆alkoxy, wherein heteroaryl, heterocycloalkyl, heterocycloalkylalkoxy, cycloalkylalkoxy or phenoxy are optionally substituted with one, two, or three substituents independently selected from halogen, haloC₁₋₆alkyl, and haloC₁₋₆alkoxy.

In one embodiment, the invention provides a compound of formula (I), or a solvate or a pharmaceutically acceptable salt thereof, wherein R¹ is selected from cyano, halogen, 5 membered heteroaryl, C₁₋₆alkyl, C₁₋₆alkoxy, 4-6 membered cycloalkylC₀₋₆alkoxy, phenoxy, 4-6 membered heterocycloalkyl, and 4-6 membered heterocycloalkylC₀₋₆alkoxy, wherein heteroaryl, heterocycloalkyl, heterocycloalkylalkoxy, cycloalkylalkoxy or phenoxy are optionally substituted with one haloC₁₋₆alkyl.

In one embodiment, the invention provides a compound of formula (I), or a solvate or a pharmaceutically acceptable salt thereof, wherein R¹ is selected from 5 membered heteroaryl, 4-6 membered cycloalkylC₀₋₆alkoxy, phenoxy, 4-6 membered heterocycloalkyl, and 4-6 membered heterocycloalkylC₀₋₆alkoxy which are optionally substituted with one haloC₁₋₆alkyl.

In one embodiment, the invention provides a compound of formula (I), or a solvate or a pharmaceutically acceptable salt thereof, wherein R¹ is selected from 5 membered heteroaryl, 4-6 membered cycloalkylC₀₋₆alkoxy, phenoxy, 4-6 membered heterocycloalkyl, and 4-6 membered heterocycloalkylC₀₋₆alkoxy which are optionally substituted with one haloC₁₋₆alkyl selected from (CH₃)₂CF—, CH₃CF₂—, CF₃—, CH₂F—, and CHF₂—.

In one embodiment, the invention provides a compound of formula (I), or a solvate or a pharmaceutically acceptable salt thereof, wherein R¹ is selected from 5 membered heteroaryl, 4-6 membered cycloalkylC₀₋₆alkoxy, phenoxy, 4-6 membered heterocycloalkyl, and 4-6 membered heterocycloalkylC₀₋₆alkoxy which are optionally substituted with one CF₃—.

In one preferred embodiment, the invention provides a compound of formula (I), or a solvate or a pharmaceutically acceptable salt thereof, wherein R¹ is cyano.

In one preferred embodiment, the invention provides a compound of formula (I), or a solvate or a pharmaceutically acceptable salt thereof, wherein R¹ is halogen selected from F—, Cl—, or Br—.

In one preferred embodiment, the invention provides a compound of formula (I), or a solvate or a pharmaceutically acceptable salt thereof, wherein R¹ is F—.

In one more preferred embodiment, the invention provides a compound of formula (I), or a solvate or a pharmaceutically acceptable salt thereof, wherein R¹ is Cl—.

In one preferred embodiment, the present invention provides a compound of formula (I), or a solvate or a pharmaceutically acceptable salt thereof, wherein R¹ is a 5 membered heteroaryl, which is a 5 membered monocyclic, aromatic group with two N-atoms, e.g. pyrazolyl or imidazolyl.

In one preferred embodiment, the present invention provides a compound of formula (I), or a solvate or a pharmaceutically acceptable salt thereof, wherein R¹ is a 5 membered heteroaryl, which is a 5 membered monocyclic, aromatic group comprising one N-atom and one O-atom, e.g. oxazolyl.

In one preferred embodiment, the present invention provides a compound of formula (I), or a solvate or a pharmaceutically acceptable salt thereof, wherein R¹ is a 5 membered heteroaryl, which is a 5 membered monocyclic, aromatic group comprising one N-atom and one S-atom, e.g. thiazolyl.

In one preferred embodiment, the present invention provides a compound of formula (I), or a solvate or a pharmaceutically acceptable salt thereof, wherein the 5 membered heteroaryl is selected from pyrazolyl, imidazolyl, oxazolyl, and thiazolyl which are optionally substituted as described herein.

In one preferred embodiment, the present invention provides a compound of formula (I), or a solvate or a pharmaceutically acceptable salt thereof, wherein the 5 membered heteroaryl is selected from pyrazolyl, imidazolyl, oxazolyl, and thiazolyl, which pyrazolyl, imidazolyl, oxazolyl, or thiazolyl are optionally substituted with one, two, or three substituents independently selected from halogen, haloC₁₋₆alkyl, and haloC₁₋₆alkoxy.

In one preferred embodiment, the present invention provides a compound of formula (I), or a solvate or a pharmaceutically acceptable salt thereof, wherein the 5 membered heteroaryl is selected from pyrazolyl, imidazolyl, oxazolyl, and thiazolyl, which pyrazolyl, imidazolyl, oxazolyl, or thiazolyl are optionally substituted with one subsituent selected from halogen, haloC₁₋₆alkyl, and haloC₁₋₆alkoxy.

In one preferred embodiment, the present invention provides a compound of formula (I), or a solvate or a pharmaceutically acceptable salt thereof, wherein the 5 membered heteroaryl is selected from pyrazolyl, imidazolyl, oxazolyl, and thiazolyl, which pyrazolyl, imidazolyl, oxazolyl, or thiazolyl are unsubstituted.

In some embodiments halogen within the substituents is selected from F—, Cl—, and Br—.

In some embodiments, halogen within the substituents is F— or Cl—.

In some preferred embodiments, halogen within the substituents is Cl—.

In some embodiments, haloC₁₋₆alkyl within the substituents is selected from (CH₃)₂CF—, CH₃CF₂—, CF₃—, CH₂F—, and CHF₂—.

In some embodiments, haloC₁₋₆alkyl within the substituents is CF₃—.

In some embodiments, haloC₁₋₆alkoxy within the substituents is selected from CHF₂O—, CH₂FO—, CF₃CH₂O—, CF₂HCH₂O—, CH₃CF₂CH₂O—, and CH₃CFHCH₂O.

In some embodiments, haloC₁₋₆alkoxy within the substituents is CHF₂O— or CF₃CH₂O—.

In some preferred embodiments, haloC₁₋₆alkoxy within the substituents is CHF₂O—.

In one preferred embodiment, the present invention provides a compound of formula (I), or a solvate or a pharmaceutically acceptable salt thereof, wherein R¹ is unsubstituted pyrazolyl.

In one embodiment, the present invention provides a compound of formula (I), or a solvate or a pharmaceutically acceptable salt thereof, wherein R¹ is pyrazolyl substituted with one, two, three substituents independently selected from halogen, haloC₁₋₆alkyl, and haloC₁₋₆alkoxy.

In one embodiment, the present invention provides a compound of formula (I), or a solvate or a pharmaceutically acceptable salt thereof, wherein R¹ is pyrazolyl substituted with one halogen selected from F—, Cl—, and Br—.

In one embodiment, the present invention provides a compound of formula (I), or a solvate or a pharmaceutically acceptable salt thereof, wherein R¹ is pyrazolyl substituted with one F—.

In one preferred embodiment, the present invention provides a compound of formula (I), or a solvate or a pharmaceutically acceptable salt thereof, wherein R¹ is pyrazolyl substituted with one Cl—.

In one embodiment, the present invention provides a compound of formula (I), or a solvate or a pharmaceutically acceptable salt thereof, wherein R¹ is pyrazolyl substituted with one haloC₁₋₆alkyl selected from (CH₃)₂CF—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is pyrazolyl substituted with one CF₃—.

In one embodiment, the present invention provides a compound of formula (I), or a solvate or a pharmaceutically acceptable salt thereof, wherein R¹ is pyrazolyl substituted with one haloC₁₋₆ alkoxy selected from CHF₂O—, CH₂FO—, CF₃CH₂O—, CF₂HCH₂O—, CH₃CF₂CH₂O—, and CH₃CFHCH₂O.

In one preferred embodiment, the present invention provides a compound of formula (I), or a solvate or a pharmaceutically acceptable salt thereof, wherein R¹ is unsubstituted imidazolyl.

In one embodiment, the present invention provides a compound of formula (I), or a solvate or a pharmaceutically acceptable salt thereof, wherein R¹ is imidazolyl substituted with one, two, three substituents independently selected from halogen, haloC₁₋₆alkyl, and haloC₁₋₆alkoxy.

In one embodiment, the present invention provides a compound of formula (I), or a solvate or a pharmaceutically acceptable salt thereof, wherein R¹ is imidazolyl substituted with one halogen selected from F—, Cl—, and Br—.

In one embodiment, the present invention provides a compound of formula (I), or a solvate or a pharmaceutically acceptable salt thereof, wherein R¹ is imidazolyl substituted with one F—.

In one preferred embodiment, the present invention provides a compound of formula (I), or a solvate or a pharmaceutically acceptable salt thereof, wherein R¹ is imidazolyl substituted with one Cl—.

In one embodiment, the present invention provides a compound of formula (I), or a solvate or a pharmaceutically acceptable salt thereof, wherein R¹ is imidazolyl substituted with one haloC₁₋₆ alkyl selected from (CH₃)₂CF—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is imidazolyl substituted with one CF₃—.

In one embodiment, the present invention provides a compound of formula (I), or a solvate or a pharmaceutically acceptable salt thereof, wherein R¹ is imidazolyl substituted with one haloC₁₋₆alkoxy selected from CHF₂O—, CH₂FO—, CF₃CH₂O—, CF₂HCH₂O—, CH₃CF₂CH₂O—, and CH₃CFHCH₂O.

In one preferred embodiment, the present invention provides a compound of formula (I), or a solvate or a pharmaceutically acceptable salt thereof, wherein R¹ is unsubstituted oxazolyl.

In one embodiment, the present invention provides a compound of formula (I), or a solvate or a pharmaceutically acceptable salt thereof, wherein R¹ is oxazolyl substituted with one, two, three substituents independently selected from halogen, haloC₁₋₆alkyl, and haloC₁₋₆alkoxy.

In one embodiment, the present invention provides a compound of formula (I), or a solvate or a pharmaceutically acceptable salt thereof, wherein R¹ is oxazolyl substituted with one halogen selected from F—, Cl—, and Br—.

In one embodiment, the present invention provides a compound of formula (I), or a solvate or a pharmaceutically acceptable salt thereof, wherein R¹ is oxazolyl substituted with one F—.

In one preferred embodiment, the present invention provides a compound of formula (I), or a solvate or a pharmaceutically acceptable salt thereof, wherein R¹ is oxazolyl substituted with one Cl—.

In one embodiment, the present invention provides a compound of formula (I), or a solvate or a pharmaceutically acceptable salt thereof, wherein R¹ is oxazolyl substituted with one haloC₁₋₆ alkyl selected from (CH₃)₂CF—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is oxazolyl substituted with one CF₃—.

In one embodiment, the present invention provides a compound of formula (I), or a solvate or a pharmaceutically acceptable salt thereof, wherein R¹ is oxazolyl substituted with one haloC₁₋₆ alkoxy selected from CHF₂O—, CH₂FO—, CF₃CH₂O—, CF₂HCH₂O—, CH₃CF₂CH₂O—, and CH₃CFHCH₂O.

In one preferred embodiment, the present invention provides a compound of formula I), or a solvate or a pharmaceutically acceptable salt thereof, wherein R¹ is unsubstituted thiazolyl.

In one embodiment, the present invention provides a compound of formula (I), or a solvate or a pharmaceutically acceptable salt thereof, wherein R¹ is thiazolyl substituted with one, two, three substituents independently selected from halogen, haloC₁₋₆alkyl, and haloC₁₋₆alkoxy.

In one embodiment, the present invention provides a compound of formula (I), or a solvate or a pharmaceutically acceptable salt thereof, wherein R¹ is thiazolyl substituted with one halogen selected from F—, Cl—, and Br—.

In one embodiment, the present invention provides a compound of formula (I), or a solvate or a pharmaceutically acceptable salt thereof, wherein R¹ is thiazolyl substituted with one F—.

In one preferred embodiment, the present invention provides a compound of formula (I), or a solvate or a pharmaceutically acceptable salt thereof, wherein R¹ is thiazolyl substituted with one Cl—.

In one embodiment, the present invention provides a compound of formula (I), or a solvate or a pharmaceutically acceptable salt thereof, wherein R¹ is thiazolyl substituted with one haloC₁₋₆ alkyl selected from (CH₃)₂CF—, CH₃CF₂—, CF₃—, CH₂F—, and CHF₂—.

In one preferred embodiment, the present invention provides a compound of formula (I), or a solvate or a pharmaceutically acceptable salt thereof, wherein R¹ is thiazolyl substituted with one CF₃—.

In one embodiment, the present invention provides a compound of formula (I), or a solvate or a pharmaceutically acceptable salt thereof, wherein R¹ is thiazolyl substituted with one haloC₁₋₆ alkoxy selected from CHF₂O—, CH₂FO—, CF₃CH₂O—, CF₂HCH₂O—, CH₃CF₂CH₂O—, and CH₃CFHCH₂O.

In one embodiment, the present invention provides a compound of formula (I), or a solvate or a pharmaceutically acceptable salt thereof, wherein R¹ is C₁₋₆alkoxy.

In one embodiment, the present invention provides a compound of formula (I), or a solvate or a pharmaceutically acceptable salt thereof, wherein R¹ is C₁₋₆alkoxy selected from CH₃O—, CH₃CH₂O—, CH₃CH₂CH₂O—, (CH₃)₂CHO—, and (CH₃)₃CO—.

In one embodiment, the present invention provides a compound of formula (I), or a solvate or a pharmaceutically acceptable salt thereof, wherein R¹ is CH₃O—, or (CH₃)₂CHO—.

In one embodiment, the present invention provides a compound of formula (I), or a solvate or a pharmaceutically acceptable salt thereof, wherein R¹ is a 3-6 membered cycloalkylC₀₋₆alkoxy which is optionally substituted with one, two, or three substituents independently selected from halogen, haloC₁₋₆alkyl, and haloC₁₋₆alkoxy.

In one embodiment, the pesent invention provides a compound of formula (I), or a solvate or a pharmaceutically acceptable salt thereof, wherein R¹ is a 3-6 membered cycloalkylC₀₋₆alkoxy selected from:

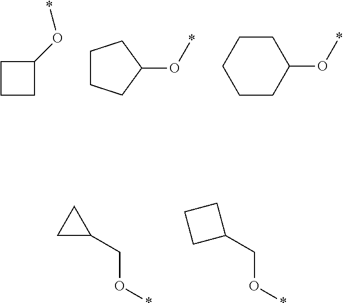

In one embodiment, the present invention provides a compound of formula (I), or a solvate or a pharmaceutically acceptable salt thereof, wherein R¹ a 3-6 membered cycloalkylC₀₋₆alkoxy having the structures:

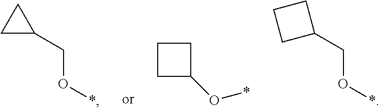

In one embodiment, the present invention provides a compound of formula (I), or a solvate or pharmaceutically acceptable salt thereof, wherein R¹ is a 3-6 membered cycloalkylC₀₋₆alkoxy having the structure

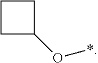

In one embodiment, the pesent invention provides a compound of formula (I), or a solvate or a pharmaceutically acceptable salt thereof, wherein R¹ is a 4-6 membered heterocycloalkylC₀₋₆alkoxy which is optionally substituted as described herein.

In one embodiment, the pesent invention provides a compound of formula (I), or a solvate or a pharmaceutically acceptable salt thereof, wherein R¹ is a 4-6 membered heterocycloalkylC₀₋₆alkoxy which is unsubstituted.

In one embodiment, the present invention provides a compound of formula (I), or a solvate or a pharmaceutically acceptable salt thereof, wherein R¹ is a 4-6 membered heterocycloalkylC₀₋₆alkoxy selected from:

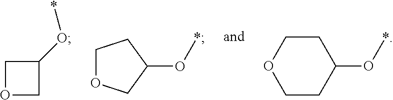

In one embodiment, the present invention provides a compound of formula (I), or a solvate or a pharmaceutically acceptable salt thereof, wherein R¹ is a 4-6 membered heterocycloalkylC₀₋₆alkoxy selected from:

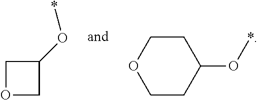

In one embodiment, the present invention provides a compound of formula (I), or a solvate or a pharmaceutically acceptable salt thereof, wherein R¹ has the structure:

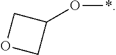

In one embodiment, the present invention provides a compound of formula (I), or a solvate or a pharmaceutically acceptable salt thereof, wherein R¹ has the structure:

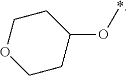

In one embodiment, the pesent invention provides a compound of formula (I), or a solvate or a pharmaceutically acceptable salt thereof, wherein R¹ is phenoxy.

In one embodiment, the present invention provides a compound of formula (I), or a solvate or a pharmaceutically acceptable salt thereof, wherein R¹ is phenoxy which is optionally substituted with one, two, or three substituents independently selected from halogen, haloC₁₋₆alkyl, and haloC₁₋₆alkoxy.

In one embodiment, the present invention provides a compound of formula (I), or a solvate or a pharmaceutically acceptable salt thereof, wherein R¹ is phenoxy which is optionally substituted as described herein, wherein halogen is selected from F— and Cl—.

In one embodiment, the present invention provides a compound of formula (I), or a solvate or a pharmaceutically acceptable salt thereof, wherein R¹ is phenoxy which is optionally substituted as described herein, wherein haloC₁₋₆alkyl is selected from (CH₃)₂FC—, CF₃CH₂—, CH₃CF₂—, CF₃—, CH₂F—, and CHF₂—.

In one embodiment, the present invention provides a compound of formula (I), or a solvate or a pharmaceutically acceptable salt thereof, wherein R¹ is phenoxy which is optionally substituted as described herein, wherein haloC₁₋₆alkoxy is selected from CHF₂O—, CF₃O—, FCH₂CFHCH₂O—, and CF₃CH₂O—.

In one embodiment, the present invention provides a compound of formula (I), or a solvate or a pharmaceutically acceptable salt thereof, wherein R¹ is phenoxy which is optionally substituted with one CF₃—, Cl—, or F—.

In one embodiment, the present invention provides a compound of formula (I), or a solvate or a pharmaceutically acceptable salt thereof, wherein R¹ is unsubstituted phenoxy.

In one embodiment, the present invention provides a compound of formula (I), or a solvate or a pharmaceutically acceptable salt thereof, wherein R¹ is unsubstituted phenoxy with the structure:

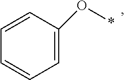 [SMILES: *Oc1ccccc1]

In one preferred embodiment, the invention provides a compound of formula (I), or a solvate or a pharmaceutically acceptable salt thereof, wherein R¹ is haloC₁₋₆alkoxy, or haloC₁₋₆alkyl.

In one preferred embodiment, the invention provides a compound of formula (I), wherein wherein R¹ is haloC₁₋₆alkoxy selected from CHF₂O—, CH₂FO—, CF₃O—, FCH₂CFHCH₂O—, CH₂FCH₂O—, CH₂FCH₂O—, and CF₃CH₂O—.

In one particularly preferred embodiment, the invention provides a compound of formula (I), wherein, wherein R¹ is CH₂FO—.

In one particularly preferred embodiment, the invention provides a compound of formula (I), wherein, wherein R¹ is CF₃O—.

In one particularly preferred embodiment, the invention provides a compound of formula (I), wherein, wherein R¹ is FCH₂CFHCH₂O—.

In one particularly preferred embodiment, the invention provides a compound of formula (I), wherein, wherein R¹ is CF₃CH₂O—.

In one particularly preferred embodiment, the invention provides a compound of formula (I), wherein, wherein R¹ is CHF₂O—.

In one particularly preferred embodiment, the invention provides a compound of formula (I), wherein, wherein R¹ is CH₂FCH₂O—.

In one embodiment, the present invention provides a compound of formula (I), or a solvate or a pharmaceutically acceptable salt thereof, wherein R¹ is haloC₁₋₆alkoxy as defined herein which is optionally substituted with C₁₋₆alkoxy as defined herein.

In one embodiment, the present invention provides a compound of formula (I), or a solvate or a pharmaceutically acceptable salt thereof, wherein R¹ is haloC₁₋₆alkoxy as defined herein which is substituted with C₁₋₆alkoxy as defined herein.

In one embodiment, the present invention provides a compound of formula (I), or a solvate or a pharmaceutically acceptable salt thereof, wherein R¹ is haloC₁₋₆alkoxy is substituted with C₁₋₆alkoxy and has the structure:

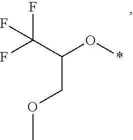 [SMILES: *OC(COC)C(F)(F)F]

In one preferred embodiment, the invention provides a compound of formula (I), or a solvate, or a pharmaceutically acceptable salt thereof, wherein R² is H.

In one preferred embodiment, the invention provides a compound of formula or (I), or a solvate, or a pharmaceutically acceptable salt thereof, wherein R² is C₁₋₆alkyl.

In this invention when R² is H or C₁₋₆alkyl in compounds of formula (I) the other substituent at this C-atom is H.

In one preferred embodiment, the invention provides a compound of formula (I), or a solvate, or a pharmaceutically acceptable salt thereof, wherein R² is C₁₋₆alkyl selected from CH₃—, CH₃CH₂—, CH₃CH₂CH₂—, (CH₃)₂CH₂—, (CH₃CH₂CH₂CH₂—), (CH₃)₂CH₂CH₂—, CH₃CH(CH₃)CH₂—, and (CH₃)₃C—.

In one preferred embodiment, the invention provides a compound of formula (I), or a solvate, or a pharmaceutically acceptable salt thereof, wherein R² is CH₃—.

In one embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, and R² is H or C₁₋₆alkyl as described herein.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, and R² is H.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are unsubstituted, and R² is H.

In one preferred embodiment, the invention provides a compound of formula (I), or a solvate, or a pharmaceutically acceptable salt thereof, wherein R¹ is halogen, and R² is H.

In one preferred embodiment, the invention provides a compound of formula (I), or a solvate, or a pharmaceutically acceptable salt thereof, wherein R¹ is Cl— and R² is H.

In one preferred embodiment, the invention provides a compound of formula (I), or a solvate, or a pharmaceutically acceptable salt thereof, wherein R¹ is cyano, and R² is H.

In one preferred embodiment, the invention provides a compound of formula (I), or a solvate, or a pharmaceutically acceptable salt thereof, wherein R¹ is 5 membered heteroaryl which is optionally substituted as described herein, and R² is H.

In one preferred embodiment, the invention provides a compound of formula (I), or a solvate, or a pharmaceutically acceptable salt thereof, wherein R¹ is 5 membered heteroaryl selected from pyrazolyl, imidazolyl, oxazolyl, and thiazolyl, which are optionally substituted as described herein, and R² is H.

In one preferred embodiment, the invention provides a compound of formula (I), or a solvate, or a pharmaceutically acceptable salt thereof, wherein R¹ is 5 membered heteroaryl selected from pyrazolyl, imidazolyl, oxazolyl, and thiazolyl, which pyrazolyl, imidazolyl, oxazolyl, or thiazolyl are optionally substituted with one subsituent selected from halogen, haloC₁₋₆alkyl, and haloC₁₋₆alkoxy, and R² is H.

In some embodiments described above, halogen within the substituents is selected from F—, Cl—, and Br—, and R² is H.

In some embodiments described above, halogen within the substituents is F— or Cl—, and R² is H.

In some embodiments as described above, halogen within the substituents is Cl—, and R² is H.

In some embodiments as described above, haloC₁₋₆alkyl within the substituents is selected from (CH₃)₂CF—, CH₃CF₂—, CF₃—, CH₂F—, and CHF₂—, and R² is H.

In some embodiments as described above, haloC₁₋₆alkyl within the substituents is CF₃—, and R² is H.

In some embodiments, haloC₁₋₆alkoxy within the substituents is selected from CHF₂O—, CH₂FO—, CF₃O—, FCH₂CFHCH₂O—, CH₂FCH₂O—, and CF₃CH₂O—, and R² is H.

In some embodiments as described above, haloC₁₋₆alkoxy within the substituents is CHF₂O— or CF₃CH₂O—, and R² is H.

In some preferred embodiments as described above, haloC₁₋₆alkoxy within the substituents is CHF₂O—, CH₂FO—, CF₃O—, FCH₂CFHCH₂O—, CH₂FCH₂O—, and R² is H.

In some preferred embodiments as described above, haloC₁₋₆alkoxy within the substituents is CHF₂O—, and R² is H.

In some preferred embodiments as described above, haloC₁₋₆alkoxy within the substituents is CH₂FO—, and R² is H.

In some preferred embodiments as described above, haloC₁₋₆alkoxy within the substituents is CF₃O—, and R² is H.

In some preferred embodiments as described above, haloC₁₋₆alkoxy within the substituents is FCH₂CFHCH₂O—, and R² is H.

In some preferred embodiments as described above, haloC₁₋₆alkoxy within the substituents is CH₂FCH₂O—, and R² is H.

In one preferred embodiment, the invention provides a compound of formula (I), or a solvate, or a pharmaceutically acceptable salt thereof, wherein R¹ is 5 membered heteroaryl selected from pyrazolyl, imidazolyl, oxazolyl, and thiazolyl which are unsubstituted, and R² is H.

In one preferred embodiment, the invention provides a compound of formula (I), or a solvate, or a pharmaceutically acceptable salt thereof, wherein R¹ is pyrazolyl which is unsubstituted, and R² is H.

In one preferred embodiment, the invention provides a compound of formula (I), or a solvate, or a pharmaceutically acceptable salt thereof, wherein R¹ is imidazolyl which is unsubstituted, and R² is H.

In one preferred embodiment, the invention provides a compound of formula (I), or a solvate, or a pharmaceutically acceptable salt thereof, wherein R¹ is oxazolyl which is unsubstituted, and R² is H.

In one preferred embodiment, the invention provides a compound of formula (I), or a solvate, or a pharmaceutically acceptable salt thereof, wherein R¹ is thiazolyl which is unsubstituted, and R² is H.

In one particularly preferred embodiment, the invention provides a compound of formula (I), or a solvate, or a pharmaceutically acceptable salt thereof, wherein R¹ is haloC₁₋₆alkoxy, and R² is H.

In one particularly preferred embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and R² is H.

In some embodiments, the haloC₁₋₆alkoxy is substituted with C₁₋₆alkoxy as described herein.

In one particularly preferred embodiment, the invention provides a compound of formula (I), or a solvate, or a pharmaceutically acceptable salt thereof, wherein R¹ is CHF₂O—, and R² is H.

In one particularly preferred embodiment, the invention provides a compound of formula (I), or a solvate, or a pharmaceutically acceptable salt thereof, wherein R¹ is CH₂FO—, and R² is H.

In one particularly preferred embodiment, the invention provides a compound of formula (I), or a solvate, or a pharmaceutically acceptable salt thereof, wherein R¹ is CF₃O—, and R² is H.

In one particularly preferred embodiment, the invention provides a compound of formula (I), or a solvate, or a pharmaceutically acceptable salt thereof, wherein R¹ is FCH₂CFHCH₂O—, and R² is H.

In one particularly preferred embodiment, the invention provides a compound of formula (I), or a solvate, or a pharmaceutically acceptable salt thereof, wherein R¹ is CH₂FCH₂O—, and R² is H.

In one preferred embodiment, the invention provides a compound of formula (I), or a solvate, or a pharmaceutically acceptable salt thereof, wherein R¹ is haloC₁₋₆alkyl.

In one preferred embodiment, the invention provides a compound of formula (I), or a solvate, or a pharmaceutically acceptable salt thereof, wherein R¹ is haloC₁₋₆alkyl selected from (CH₃)₂FC—, CF₃CH₂—, CH₃CF₂—, CF₃—, CH₂F—, and CHF₂—, and R² is H.

In one preferred embodiment, the invention provides a compound of formula (I), or a solvate, or a pharmaceutically acceptable salt thereof, wherein R¹ is (CH₃)₂FC—, and R² is H.

In one preferred embodiment, the invention provides a compound of formula (I), or a solvate, or a pharmaceutically acceptable salt thereof, wherein R¹ is CF₃CH₂—, and R² is H.

In one preferred embodiment, the invention provides a compound of formula (I), or a solvate, or a pharmaceutically acceptable salt thereof, wherein R¹ is CH₃CF₂—, and R² is H.

In one preferred embodiment, the invention provides a compound of formula (I), or a solvate, or a pharmaceutically acceptable salt thereof, wherein R¹ is CF₃—, and CHF₂—, and R² is H.

In one preferred embodiment, the invention provides a compound of formula (I), or a solvate, or a pharmaceutically acceptable salt thereof, wherein R¹ is CHF₂—, and R² is H.

In one preferred embodiment, the invention provides a compound of formula (I), or a solvate, or a pharmaceutically acceptable salt thereof, wherein R¹ is a 4-6 membered heterocycloalkylC₀₋₆alkoxy, and R² is H.

In one preferred embodiment, the invention provides a compound of formula (I), or a solvate, or a pharmaceutically acceptable salt thereof, wherein R¹ is a 4-6 membered heterocycloalkylC₀₋₆alkoxy selected from:

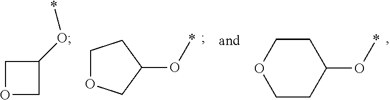

### and R² is H.

In one preferred embodiment, the invention provides a compound of formula (I), or a solvate, or a pharmaceutically acceptable salt thereof, wherein R¹ is

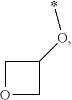 [SMILES: *OC1COC1]

### and R² is H.

In one preferred embodiment, the invention provides a compound of formula (I), or a solvate, or a pharmaceutically acceptable salt thereof, wherein R¹ is

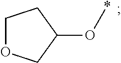 [SMILES: *OC1CCOC1]

### and R² is H.

In one preferred embodiment, the invention provides a compound of formula (I), or a solvate, or a pharmaceutically acceptable salt thereof, wherein R¹ is

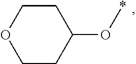 [SMILES: *OC1CCOCC1]

### and R² is H.

In one embodiment, the present invention provides a compound of formula (I), or a solvate or a pharmaceutically acceptable salt thereof, wherein R¹ is phenoxy which is optionally substituted with one, two, or three substituents independently selected from halogen, haloC₁₋₆alkyl, and haloC₁₋₆alkoxy, and R² is H.

In one embodiment, the present invention provides a compound of formula (I), or a solvate or a pharmaceutically acceptable salt thereof, wherein R¹ is phenoxy which is unsubstituted, and R² is H.

In one embodiment, the present invention provides a compound of formula (I), or a solvate or a pharmaceutically acceptable salt thereof, wherein R¹ is a 3-6 membered cycloalkylC₀₋₆alkoxy which is optionally substituted with one, two, or three substituents independently selected from halogen, haloC₁₋₆alkyl, and haloC₁₋₆alkoxy, and R² is H.

In one embodiment, the pesent invention provides a compound of formula (I), or a solvate or a pharmaceutically acceptable salt thereof, wherein R¹ is a 3-6 membered cycloalkylC₀₋₆alkoxy selected from:

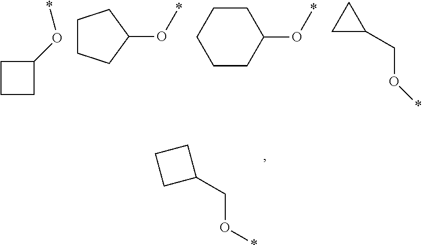

### and R² is H.

In one embodiment, the present invention provides a compound of formula (I), or a solvate or a pharmaceutically acceptable salt thereof, wherein R¹ a 3-6 membered cycloalkylC₀₋₆alkoxy having the structure:

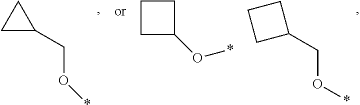

### and R² is H.

In one embodiment, the present invention provides a compound of formula (I), or a solvate or pharmaceutically acceptable salt thereof, wherein R¹ is a 3-6 membered cycloalkylC₀₋₆alkoxy having the structure

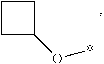 [SMILES: *OC1CCC1]

### and R² is H.

In one embodiment, the pesent invention provides a compound of formula (I), or a solvate or a pharmaceutically acceptable salt thereof, wherein R¹ is a 4-6 membered heterocycloalkyl which is optionally substituted as described herein, and R² is H.

In one embodiment, the pesent invention provides a compound of formula (I), or a solvate or a pharmaceutically acceptable salt thereof, wherein R¹ is a 4-6 membered heterocycloalkyl which is unsubstituted, and R² is H.

In one embodiment, the present invention provides a compound of formula (I), or a solvate or a pharmaceutically acceptable salt thereof, wherein R¹ is a 4-6 membered heterocycloalkyl selected from:

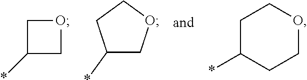

### and R² is H.

In one embodiment, the present invention provides a compound of formula (I), or a solvate or a pharmaceutically acceptable salt thereof, wherein R¹ is

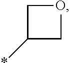 [SMILES: *C1COC1]

### and R² is H.

In one embodiment, the present invention provides a compound of formula (I), or a solvate or a pharmaceutically acceptable salt thereof, wherein R¹ is C₁₋₆alkyl or haloC₁₋₆alkoxy, both as described herein, R² is C₁₋₆alkyl as described herein.

In one embodiment, the present invention provides a compound of formula (I), or a solvate or a pharmaceutically acceptable salt thereof, wherein R¹ is C₁₋₆alkyl or haloC₁₋₆alkoxy, both as described herein, and R² is CH₃—.

In one embodiment, the invention provides a compound of formula (I), or a solvate, or a pharmaceutically acceptable salt thereof, wherein R³ is H or C₁₋₆alkyl.

In one preferred embodiment, the invention provides a compound of formula (I), or a solvate, or a pharmaceutically acceptable salt thereof, wherein R³ is H.

In one embodiment, the invention provides a compound of formula (I), or a solvate, or a pharmaceutically acceptable salt thereof, wherein R³ is C₁₋₆alkyl.

In one embodiment, the invention provides a compound of formula (I), or a solvate, or a pharmaceutically acceptable salt thereof, wherein R³ is CH₃—.

In one embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, and R³ is C₁₋₆alkyl selected from CH₃—, CH₃CH₂—, CH₃CH₂CH₂—, (CH₃)₂CH₂—, (CH₃CH₂CH₂CH₂—), (CH₃)₂CH₂CH₂—, CH₃CH(CH₃)CH₂—, and (CH₃)₃C—.

In one embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkyl C₀₋₆alkoxy, 4-6 membered heterocycloalkyl, 4-6 membered heterocycloalkyl C₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, and R³ is CH₃—.

In one embodiment, the invention provides a compound of formula (I), or a solvate, or a pharmaceutically acceptable salt thereof, wherein R¹ is haloC₁₋₆alkoxy as described herein, R² is H, and R³ is CH₃—.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkyl C₀₋₆alkoxy, 4-6 membered heterocycloalkyl, 4-6 membered heterocycloalkyl C₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, and R³ is H.

In one embodiment, the invention provides a compound of formula (I), or a solvate, or a pharmaceutically acceptable salt thereof, wherein R¹ is cyano, R² is H, and R³ is H.

In one preferred embodiment, the invention provides a compound of formula (I), or a solvate, or a pharmaceutically acceptable salt thereof, wherein R¹ is haloC₁₋₆alkyl as described herein, R² is H, and R³ is H.

In one preferred embodiment, the invention provides a compound of formula (I), or a solvate, or a pharmaceutically acceptable salt thereof, wherein R¹ is halogen as described herein, R² is H, and R³ is H.

In one preferred embodiment, the invention provides a compound of formula (I), or a solvate, or a pharmaceutically acceptable salt thereof, wherein R¹ is 5 membered heteroaryl which is optionally substituted with one, two, or three substituents independently selected from halogen, haloC₁₋₆alkyl, and haloC₁₋₆alkoxy, R² is H, and R³ is H.

In one preferred embodiment, the invention provides a compound of formula (I), or a solvate, or a pharmaceutically acceptable salt thereof, wherein R¹ is C₁₋₆alkyl as described herein, R² is H, and R³ is H.

In one preferred embodiment, the invention provides a compound of formula (I), or a solvate, or a pharmaceutically acceptable salt thereof, wherein R¹ is C₁₋₆alkoxy as described herein, R² is H, and R³ is H.

In one preferred embodiment, the invention provides a compound of formula (I), or a solvate, or a pharmaceutically acceptable salt thereof, wherein R¹ is 3-6 membered cycloalkylC₀₋₆alkoxy which is optionally substituted with one, two, or three substituents independently selected from halogen, haloC₁₋₆alkyl, and haloC₁₋₆alkoxy, R² is H, and R³ is H.

In one preferred embodiment, the invention provides a compound of formula (I), or a solvate, or a pharmaceutically acceptable salt thereof, wherein R¹ is 3-6 membered cycloalkylC₀₋₆alkoxy which is unsubstituted, R² is H, and R³ is H.

In one preferred embodiment, the invention provides a compound of formula (I), or a solvate, or a pharmaceutically acceptable salt thereof, wherein R¹ is 4-6 membered heterocycloalkyl, which is optionally substituted with one, two, or three substituents independently selected from halogen, haloC₁₋₆alkyl, and haloC₁₋₆alkoxy, R² is H, and R³ is H.

In one preferred embodiment, the invention provides a compound of formula (I), or a solvate, or a pharmaceutically acceptable salt thereof, wherein R¹ is 4-6 membered heterocycloalkyl, which is unsubstituted, R² is H, and R³ is H.

In one preferred embodiment, the invention provides a compound of formula (I), or a solvate, or a pharmaceutically acceptable salt thereof, wherein R¹ is 4-6 membered heterocycloalkylC₀₋₆alkoxy, which is optionally substituted with one, two, or three substituents independently selected from halogen, haloC₁₋₆alkyl, and haloC₁₋₆alkoxy, R² is H, and R³ is H.

In one preferred embodiment, the invention provides a compound of formula (I), or a solvate, or a pharmaceutically acceptable salt thereof, wherein R¹ is 4-6 membered heterocycloalkylC₀₋₆alkoxy, which is unsubstituted, R² is H, and R³ is H.

In one preferred embodiment, the invention provides a compound of formula (I), or a solvate, or a pharmaceutically acceptable salt thereof, wherein R¹ is phenoxy which is optionally substituted with one, two, or three substituents independently selected from halogen, haloC₁₋₆alkyl, and haloC₁₋₆alkoxy, R² is H, and R³ is H.

In one preferred embodiment, the invention provides a compound of formula (I), or a solvate, or a pharmaceutically acceptable salt thereof, wherein R¹ is phenoxy which unsubstituted, R² is H, and R³ is H.

In one particularly preferred embodiment, the invention provides a compound of formula (I), or a solvate, or a pharmaceutically acceptable salt thereof, wherein R¹ is haloC₁₋₆alkoxy as described herein, R² is H, and R³ is H.

In one particularly preferred embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is H, and R³ is H.

In one particularly preferred embodiment, the invention provides a compound of formula (I), or a solvate, or a pharmaceutically acceptable salt thereof, wherein R¹ is haloC₁₋₆alkoxy is CHF₂O—, R² is H, and R³ is H.

In one particularly preferred embodiment, the invention provides a compound of formula (I), or a solvate, or a pharmaceutically acceptable salt thereof, wherein R¹ is CH₂FO—, R² is H, and R³ is H.

In one particularly preferred embodiment, the invention provides a compound of formula (I), or a solvate, or a pharmaceutically acceptable salt thereof, wherein R¹ is CF₃O—, R² is H, and R³ is H.

In one particularly preferred embodiment, the invention provides a compound of formula (I), or a solvate, or a pharmaceutically acceptable salt thereof, wherein R¹ is FCH₂CFHCH₂O—, R² is H, and R³ is H.

In one particularly preferred embodiment, the invention provides a compound of formula (I), or a solvate, or a pharmaceutically acceptable salt thereof, wherein R¹ is CH₂FCH₂O—, R² is H, and R³ is H.

In one particularly preferred embodiment, the invention provides a compound of formula (I), or a solvate, or a pharmaceutically acceptable salt thereof, wherein R¹ is CF₃CH₂O—, R² is H, and R³ is H.

In one embodiment, the invention provides a compound of formula (I), or a solvate, or a pharmaceutically acceptable salt thereof, wherein R¹ is haloC₁₋₆alkoxy as described herein, R² is C₁₋₆alkyl, and R³ is H.

In one embodiment, the invention provides a compound of formula (I), or a solvate, or a pharmaceutically acceptable salt thereof, wherein R¹ is haloC₁₋₆alkoxy as described herein, R² is CH₃—, and R³ is H.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is CH₃— and R³ is H.

In one embodiment, the invention provides a compound of formula (I), or a solvate, or a pharmaceutically acceptable salt thereof, wherein R¹ is CHF₂O—, R² is CH₃—, and R³ is H.

In one embodiment, the invention provides a compound of formula (I), or a solvate, or a pharmaceutically acceptable salt thereof, wherein R¹ is CH₂FO—, R² is CH₃—, and R³ is H.

In one embodiment, the invention provides a compound of formula (I), or a solvate, or a pharmaceutically acceptable salt thereof, wherein R¹ is CF₃O—, R² is CH₃—, and R³ is H.

In one embodiment, the invention provides a compound of formula (I), or a solvate, or a pharmaceutically acceptable salt thereof, wherein R¹ is FCH₂CFHCH₂O—, R² is CH₃—, and R³ is H.

In one embodiment, the invention provides a compound of formula (I), or a solvate, or a pharmaceutically acceptable salt thereof, wherein R¹ is CH₂FCH₂O—, R² is CH₃—, and R³ is H.

In one embodiment, the invention provides a compound of formula (I), or a solvate, or a pharmaceutically acceptable salt thereof, wherein R¹ is CF₃CH₂O—, R² is CH₃—, and R³ is H.

In one embodiment, the invention provides a compound of formula (I), or a solvate, or a pharmaceutically acceptable salt thereof, wherein (i) one of R⁴ and R⁵ is H and the other is halogen, (ii) R⁴ and R⁵ are both H, or (iii) R⁴ and R⁵ are halogen.

In one embodiment, the invention provides a compound of formula (I), or a solvate, or a pharmaceutically acceptable salt thereof, wherein R⁴ and R⁵ are both H or both halogen.

In one preferred embodiment, the invention provides a compound of formula (I), or a solvate, or a pharmaceutically acceptable salt thereof, wherein R⁴ and R⁵ are both H.

In one embodiment, the invention provides a compound of formula (I), or a solvate, or a pharmaceutically acceptable salt thereof, wherein R⁴ and R⁵ are both halogen.

In one embodiment, the invention provides a compound of formula (I), or a solvate, or a pharmaceutically acceptable salt thereof, wherein R⁴ and R⁵ are both halogen selected from F—, and Cl—.

In one embodiment, the invention provides a compound of formula (I), or a solvate, or a pharmaceutically acceptable salt thereof, wherein R⁴ and R⁵ are both F—.

In one embodiment, the invention provides a compound of formula (I), or a solvate, or a pharmaceutically acceptable salt thereof, wherein R⁴ is H and R⁵ is F—.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, and R⁴ and R⁵ are both H.

In one preferred embodiment, the invention provides a compound of formula (I), or a solvate, or a pharmaceutically acceptable salt thereof, wherein R¹ is cyano, R² is H, R³ is H, and R⁴ and R⁵ are both H.

In one preferred embodiment, the invention provides a compound of formula (I), or a solvate, or a pharmaceutically acceptable salt thereof, wherein R¹ is haloC₁₋₆alkyl as described herein, R² is H, R³ is H, and R⁴ and R⁵ are both H.

In one preferred embodiment, the invention provides a compound of formula (I), or a solvate, or a pharmaceutically acceptable salt thereof, wherein R¹ is halogen as described herein, R² is H, R³ is H, and R⁴ and R⁵ are both H.

In one preferred embodiment, the invention provides a compound of formula (I), or a solvate, or a pharmaceutically acceptable salt thereof, wherein R¹ is 5 membered heteroaryl as described herein which is optionally substituted with one, two, or three substituents independently selected from halogen, haloC₁₋₆alkyl, and haloC₁₋₆alkoxy, R² is H, R³ is H, and R⁴ and R⁵ are both H.

In one preferred embodiment, the invention provides a compound of formula (I), or a solvate, or a pharmaceutically acceptable salt thereof, wherein R¹ is C₁₋₆alkyl as described herein, R² is H, R³ is H, and R⁴ and R⁵ are both H.

In one preferred embodiment, the invention provides a compound of formula (I), or a solvate, or a pharmaceutically acceptable salt thereof, wherein R¹ is C₁₋₆alkoxy as described herein, R² is H, R³ is H, and R⁴ and R⁵ are both H.

In one preferred embodiment, the invention provides a compound of formula (I), or a solvate, or a pharmaceutically acceptable salt thereof, wherein R¹ is 3-6 membered cycloalkyC₀₋₆alkoxy which is optionally substituted with one, two, or three substituents independently selected from halogen, haloC₁₋₆alkyl, and haloC₁₋₆alkoxy, R² is H, R³ is H, and R⁴ and R⁵ are both H.

In one preferred embodiment, the invention provides a compound of formula (I), or a solvate, or a pharmaceutically acceptable salt thereof, wherein R¹ is 3-6 membered cycloalkylC₀₋₆alkoxy which is unsubstituted, R² is H, R³ is H, and R⁴ and R⁵ are both H.

In one preferred embodiment, the invention provides a compound of formula (I), or a solvate, or a pharmaceutically acceptable salt thereof, wherein R¹ is 4-6 membered heterocycloalkyl, which is optionally substituted with one, two, or three substituents independently selected from halogen, haloC₁₋₆alkyl, and haloC₁₋₆alkoxy, R² is H, R³ is H, and R⁴ and R⁵ are both H.

In one preferred embodiment, the invention provides a compound of formula (I), or a solvate, or a pharmaceutically acceptable salt thereof, wherein R¹ is 4-6 membered heterocycloalkyl, which is unsubstituted, R² is H, R³ is H, and R⁴ and R⁵ are both H.

In one preferred embodiment, the invention provides a compound of formula (I), or a solvate, or a pharmaceutically acceptable salt thereof, wherein R¹ is 4-6 membered heterocycloalkylC₀₋₆alkoxy, which is optionally substituted with one, two, or three substituents independently selected from halogen, haloC₁₋₆alkyl, and haloC₁₋₆alkoxy, R² is H, R³ is H, and R⁴ and R⁵ are both H.

In one embodiment, the invention provides a compound of formula (I), or a solvate, or a pharmaceutically acceptable salt thereof, wherein R¹ is 4-6 membered heterocycloalkylC₀₋₆alkoxy, which is unsubstituted, R² is H, R³ is H, and R⁴ and R⁵ are both H.

In one preferred embodiment, the invention provides a compound of formula (I), or a solvate, or a pharmaceutically acceptable salt thereof, wherein R¹ is phenoxy which is optionally substituted with one, two, or three substituents independently selected from halogen, haloC₁₋₆alkyl, and haloC₁₋₆alkoxy, R² is H, R³ is H, and R⁴ and R⁵ are both H.

In one preferred embodiment, the invention provides a compound of formula (I), or a solvate, or a pharmaceutically acceptable salt thereof, wherein R¹ is phenoxy which unsubstituted, R² is H, R³ is H, and R⁴ and R⁵ are both H.

In one particularly preferred embodiment, the invention provides a compound of formula (I), or a solvate, or a pharmaceutically acceptable salt thereof, wherein R¹ is haloC₁₋₆alkoxy as described herein, R² is H, R³ is H, and R⁴ and R⁵ are both H.

In one particularly preferred embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is haloC₁₋₆alkoxy is CHF₂O—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is CH₂FO—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is CF₃O—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is FCH₂CFHCH₂O—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is CH₂FCH₂O—, R² is H, R³ is H, and R⁴ and R⁵ are both H.

In one particularly preferred embodiment, the invention provides a compound of formula (I), or a solvate, or a pharmaceutically acceptable salt thereof, wherein R¹ is CF₃CH₂O—, R² is H, R³ is H, and R⁴ and R⁵ are both H.

In one embodiment, the invention provides a compound of formula (I), or a solvate, or a pharmaceutically acceptable salt thereof, wherein R¹ is haloC₁₋₆alkoxy as described herein, R² is C₁₋₆alkyl, R³ is H, and R⁴ and R⁵ are both H.

In one embodiment, the invention provides a compound of formula (I), or a solvate, or a pharmaceutically acceptable salt thereof, wherein R¹ is haloC₁₋₆alkoxy as described herein, R² is CH₃—, R³ is H, and R⁴ and R⁵ are both H.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is CH₃—, R³ is H, and R⁴ and R⁵ are both H.

In one embodiment, the invention provides a compound of formula (I), or a solvate, or a pharmaceutically acceptable salt thereof, wherein R¹ is CHF₂O—, R² is CH₃—, R³ is H, and R⁴ and R⁵ are both H.

In one embodiment, the invention provides a compound of formula (I), or a solvate, or a pharmaceutically acceptable salt thereof, wherein R¹ is CH₂FO—, R² is CH₃—, R³ is H, and R⁴ and R⁵ are both H.

In one embodiment, the invention provides a compound of formula (I), or a solvate, or a pharmaceutically acceptable salt thereof, wherein R¹ is CF₃O—, R² is CH₃—, R³ is H, and R⁴ and R⁵ are both H.

In one embodiment, the invention provides a compound of formula (I), or a solvate, or a pharmaceutically acceptable salt thereof, wherein R¹ is FCH₂CFHCH₂O—, R² is CH₃—, R³ is H, and R⁴ and R⁵ are both H.

In one embodiment, the invention provides a compound of formula (I), or a solvate, or a pharmaceutically acceptable salt thereof, wherein R¹ is CH₂FCH₂O—, R² is CH₃—, R³ is H, and R⁴ and R⁵ are both H.

In one embodiment, the invention provides a compound of formula (I), or a solvate, or a pharmaceutically acceptable salt thereof, wherein R¹ is CF₃CH₂O—, R² is CH₃—, R³ is H, and R⁴ and R⁵ are both H.

In one embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is cyano, R² is H, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkyl as described herein, R² is H, R³ is H, and R⁴ and R⁵ are both F—.

In one embodiment, the invention provides a compound of formula (I), or a solvate, or a pharmaceutically acceptable salt thereof, wherein R¹ is halogen as described herein, R² is H, R³ is H, and R⁴ and R⁵ are both F—.

In one embodiment, the invention provides a compound of formula (I), or a solvate, or a pharmaceutically acceptable salt thereof, wherein R¹ is 5 membered heteroaryl as described herein, which is optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is C₁₋₆alkyl as described herein, R² is H, R³ is H, and R⁴ and R⁵ are both F—.

In one embodiment, the invention provides a compound of formula (I), or a solvate, or a pharmaceutically acceptable salt thereof, wherein R¹ is C₁₋₆alkoxy as described herein, R² is H, R³ is H, and R⁴ and R⁵ are both F—.

In one embodiment, the invention provides a compound of formula (I), or a solvate, or a pharmaceutically acceptable salt thereof, wherein R¹ is 3-6 membered cycloalkylC₀₋₆alkoxy which is optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is 3-6 membered cycloalkylC₀₋₆alkoxy which is unsubstituted, R² is H, R³ is H, and R⁴ and R⁵ are both F—.

In one embodiment, the invention provides a compound of formula (I), or a solvate, or a pharmaceutically acceptable salt thereof, wherein R¹ is 4-6 membered heterocycloalkyl, which is optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is 4-6 membered heterocycloalkyl, which is unsubstituted, R² is H, R³ is H, and R⁴ and R⁵ are both F—.

In one embodiment, the invention provides a compound of formula (I), or a solvate, or a pharmaceutically acceptable salt thereof, wherein R¹ is 4-6 membered heterocycloalkylC₀₋₆alkoxy, which is optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is 4-6 membered heterocycloalkylC₀₋₆alkoxy, which is unsubstituted, R² is H, R³ is H, and R⁴ and R⁵ are both F—.

In one embodiment, the invention provides a compound of formula (I), or a solvate, or a pharmaceutically acceptable salt thereof, wherein R¹ is phenoxy which is optionally substituted with one, two, or three substituents independently selected from halogen, haloC₁₋₆alkyl, and haloC₁₋₆alkoxy, R² is H, R³ is H, and R⁴ and R⁵ are both F—.

In one embodiment, the invention provides a compound of formula (I), or a solvate, or a pharmaceutically acceptable salt thereof, wherein R¹ is phenoxy which unsubstituted, R² is H, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkoxy as described herein, R² is H, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is H, R³ is H, and R⁴ and R⁵ are both F—.

In one embodiment, the invention provides a compound of formula (I), or a solvate, or a pharmaceutically acceptable salt thereof, wherein R¹ is CHF₂O—, R² is H, R³ is H, and R⁴ and R⁵ are both F—.

In one embodiment, the invention provides a compound of formula (I), or a solvate, or a pharmaceutically acceptable salt thereof, wherein R¹ is CH₂FO—, R² is H, R³ is H, and R⁴ and R⁵ are both F—.

In one embodiment, the invention provides a compound of formula (I), or a solvate, or a pharmaceutically acceptable salt thereof, wherein R¹ is CF₃O—, R² is H, R³ is H, and R⁴ and R⁵ are both F—.

In one embodiment, the invention provides a compound of formula (I), or a solvate, or a pharmaceutically acceptable salt thereof, wherein R¹ is FCH₂CFHCH₂O—, R² is H, R³ is H, and R⁴ and R⁵ are both F—.

In one embodiment, the invention provides a compound of formula (I), or a solvate, or a pharmaceutically acceptable salt thereof, wherein R¹ is CH₂FCH₂O—, R² is H, R³ is H, and R⁴ and R⁵ are both F—.

In one embodiment, the invention provides a compound of formula (I), or a solvate, or a pharmaceutically acceptable salt thereof, wherein R¹ is CF₃CH₂O—, R² is H, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkoxy as described herein, R² is C₁₋₆alkyl, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkoxy as described herein, R² is CH₃—, R³ is H, and R⁴ and R⁵ are both F—.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is CHF₂O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is CH₂FO—, R² is CH₃—, R³ is H, and R⁴ and R⁵ are both F—.

In one embodiment, the invention provides a compound of formula (I), or a solvate, or a pharmaceutically acceptable salt thereof, wherein R¹ is CF₃O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is FCH₂CFHCH₂O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is CH₂FCH₂O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is CF₃CH₂O—, R² is CH₃—, R³ is H, and R⁴ and R⁵ are both F—.

In one embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is cyano, R² is H, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkyl as described herein, R² is H, R³ is H, R⁴ is H, and R⁵ is F—.

In one embodiment, the invention provides a compound of formula (I), or a solvate, or a pharmaceutically acceptable salt thereof, wherein R¹ is halogen as described herein, R² is H, R³ is H, R⁴ is H, and R⁵ is F—.

In one embodiment, the invention provides a compound of formula (I), or a solvate, or a pharmaceutically acceptable salt thereof, wherein R¹ is 5 membered heteroaryl which is optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is C₁₋₆alkyl as described herein, R² is H, R³ is H, R⁴ is H, and R⁵ is F—.

In one embodiment, the invention provides a compound of formula (I), or a solvate, or a pharmaceutically acceptable salt thereof, wherein R¹ is C₁₋₆alkoxy as described herein, R² is H, R³ is H, R⁴ is H, and R⁵ is F—.

In one embodiment, the invention provides a compound of formula (I), or a solvate, or a pharmaceutically acceptable salt thereof, wherein R¹ is 3-6 membered cycloalkylC₀₋₆alkoxy which is optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is 3-6 membered cycloalkylC₀₋₆alkoxy which is unsubstituted, R² is H, R³ is H, R⁴ is H and R⁵ is F—.

In one embodiment, the invention provides a compound of formula (I), or a solvate, or a pharmaceutically acceptable salt thereof, wherein R¹ is 4-6 membered heterocycloalkyl, which is optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is 4-6 membered heterocycloalkyl, which is unsubstituted, R² is H, R³ is H, R⁴ is H, and R⁵ is F—.

In one embodiment, the invention provides a compound of formula (I), or a solvate, or a pharmaceutically acceptable salt thereof, wherein R¹ is 4-6 membered heterocycloalkylC₀₋₆alkoxy, which is optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is 4-6 membered heterocycloalkylC₀₋₆alkoxy, which is unsubstituted, R² is H, R³ is H, R⁴ is H, and R⁵ is F—.

In one embodiment, the invention provides a compound of formula (I), or a solvate, or a pharmaceutically acceptable salt thereof, wherein R¹ is phenoxy which is optionally substituted with one, two, or three substituents independently selected from halogen, haloC₁₋₆alkyl, and haloC₁₋₆alkoxy, R² is H, R³ is H, R⁴ is H, and R⁵ is F—.

In one embodiment, the invention provides a compound of formula (I), or a solvate, or a pharmaceutically acceptable salt thereof, wherein R¹ is phenoxy which unsubstituted, R² is H, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkoxy as described herein, R² is H, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is H, R³ is H, R⁴ is H, and R⁵ is F—.

In one embodiment, the invention provides a compound of formula (I), or a solvate, or a pharmaceutically acceptable salt thereof, wherein R¹ is CHF₂O—, R² is H, R³ is H, R⁴ is H, and R⁵ is F—.

In one embodiment, the invention provides a compound of formula (I), or a solvate, or a pharmaceutically acceptable salt thereof, wherein R¹ is CH₂FO—, R² is H, R³ is H, R⁴ is H, and R⁵ is F—.

In one embodiment, the invention provides a compound of formula (I), or a solvate, or a pharmaceutically acceptable salt thereof, wherein R¹ is CF₃O—, R² is H, R³ is H, R⁴ is H, and R⁵ is F—.

In one embodiment, the invention provides a compound of formula (I), or a solvate, or a pharmaceutically acceptable salt thereof, wherein R¹ is FCH₂CFHCH₂O—, R² is H, R³ is H, R⁴ is H, and R⁵ is F—.

In one embodiment, the invention provides a compound of formula (I), or a solvate, or a pharmaceutically acceptable salt thereof, wherein R¹ is CH₂FCH₂O—, R² is H, R³ is H, R⁴ is H, and R⁵ is F—.

In one embodiment, the invention provides a compound of formula (I), or a solvate, or a pharmaceutically acceptable salt thereof, wherein R¹ is CF₃CH₂O—, R² is H, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkoxy as described herein, R² is C₁₋₆alkyl, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkoxy as described herein, R² is CH₃—, R³ is H, R⁴ is H, and R⁵ is F—.

In one embodiment, the invention provides a compound of formula (I), or a solvate, or a pharmaceutically acceptable salt thereof, wherein R¹ is haloC₁₋₆alkoxy selected from CHF₂O—, CH₂FO—, CF₃O—, FCH₂CFHCH₂O—, CH₂FCH₂O—, and CF₃CH₂O—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is CHF₂O—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is CH₂FO—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is CF₃O—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is FCH₂CFHCH₂O—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is CH₂FCH₂O—, R² is CH₃—, R³ is H, R⁴ is H, and R⁵ is F—.

In one embodiment, the invention provides a compound of formula (I), or a solvate, or a pharmaceutically acceptable salt thereof, wherein R¹ is CF₃CH₂O—, R² is CH₃—, R³ is H, R⁴ is H, and R⁵ is F—.

In one preferred embodiment, the invention provides a compound of formula (III), or a solvate, or a pharmaceutically acceptable salt thereof:

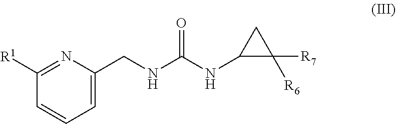

wherein R¹, R⁶ and R⁷ are as described herein.

In one particularly preferred embodiment, the invention provides a compound of formula (IV), or a solvate, or a pharmaceutically acceptable salt thereof:

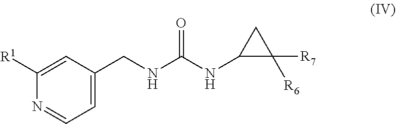

wherein R¹, R⁶ and R⁷ are as described herein.

In one particularly preferred embodiment, the invention provides a compound of formula (V), or a solvate, or a pharmaceutically acceptable salt thereof:

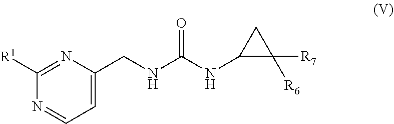

wherein R¹, R⁶ and R⁷ are as described herein.

In one embodiment, the invention provides a compound of formula (I), or a solvate, or a pharmaceutically acceptable salt thereof, wherein R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy.

In one preferred embodiment, the invention provides a compound of formula (I), or a solvate, or a pharmaceutically acceptable salt thereof, wherein R⁶ is phenyl which is optionally substituted as described herein.

In one preferred embodiment, the invention provides a compound of formula (I), or a solvate, or a pharmaceutically acceptable salt thereof, wherein R⁶ is unsubstituted phenyl.

In one preferred embodiment, the invention provides a compound of formula (I), or a solvate, or a pharmaceutically acceptable salt thereof, wherein R⁶ is a saturated monocyclic 3-6 membered saturated monocyclic cycloalkyl which is optionally substituted as described herein.

In one preferred embodiment, the invention provides a compound of formula (I), or a solvate, or a pharmaceutically acceptable salt thereof, wherein R⁶ is a saturated monocyclic 3-6 membered cycloalkyl, selected from cyclopropyl, cyclobutyl, cyclopentyl and cyclohexyl, which are optionally substituted as described herein.

In one more preferred embodiment, the invention provides a compound of formula (I), or a solvate, or a pharmaceutically acceptable salt thereof, wherein R⁶ is a saturated monocyclic 3-6 membered cycloalkyl, selected from cyclopropyl, cyclobutyl, cyclopentyl and cyclohexyl which are unsubstituted.

In one more preferred embodiment, the invention provides a compound of formula (I), or a solvate, or a pharmaceutically acceptable salt thereof, wherein R⁶ is cyclohexyl which is unsubstituted.

In one embodiment, the invention provides a compound of formula (I), or a solvate, or a pharmaceutically acceptable salt thereof, wherein R⁶ is a C₁₋₆alkyl.

In one embodiment, the invention provides a compound of formula (I), or a solvate, or a pharmaceutically acceptable salt thereof, wherein R⁶ is a C₁₋₆alkyl selected from CH₃—, CH₃CH₂—, CH₃CH₂CH₂—, (CH₃)₂CH₂—, (CH₃CH₂CH₂CH₂—), (CH₃)₂CH₂CH₂—, CH₃CH(CH₃)CH₂—, and (CH₃)₃C—.

In one embodiment, the invention provides a compound of formula (I), or a solvate, or a pharmaceutically acceptable salt thereof, wherein R⁶ is CH₃—.

In one embodiment, the invention provides a compound of formula (I), or a solvate, or a pharmaceutically acceptable salt thereof, wherein R⁶ is a 4-6 membered heterocycloalkyl.

In one embodiment, the invention provides a compound of formula (I), or a solvate, or a pharmaceutically acceptable salt thereof, wherein R⁶ is a 4-6 membered heterocycloalkyl as described herein, which is optionally substituted with one, two, or three substituents independently selected from halogen, haloC₁₋₆alkyl, and haloC₁₋₆alkoxy.

In one embodiment, the invention provides a compound of formula (I), or a solvate, or a pharmaceutically acceptable salt thereof, wherein R⁶ is a 4-6 membered heterocycloalkyl as described herein, which is unsubstituted.

In one embodiment, the present invention provides a compound of formula (I), or a solvate or a pharmaceutically acceptable salt thereof, wherein R⁶ is a 4-6 membered heterocycloalkyl selected from:

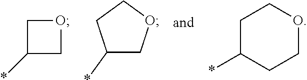

In one embodiment, the present invention provides a compound of formula (I), or a solvate or a pharmaceutically acceptable salt thereof, wherein R⁶ is:

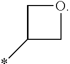 [SMILES: *C1COC1]

In one embodiment, the present invention provides a compound of formula (I), or a solvate or a pharmaceutically acceptable salt thereof, wherein R⁶ is:

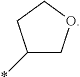 [SMILES: *C1CCOC1]

In one embodiment, the present invention provides a compound of formula (I), or a solvate or a pharmaceutically acceptable salt thereof, wherein R⁶ is:

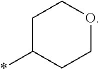 [SMILES: *C1CCOCC1]

In one particularly preferred embodiment, the invention provides a compound of formula (I), or a solvate, or a pharmaceutically acceptable salt thereof, wherein R⁶ is a haloC₁₋₆alkyl.

In one particularly preferred embodiment, the invention provides a compound of formula (I), or a solvate, or a pharmaceutically acceptable salt thereof, wherein R⁶ is a haloC₁₋₆alkyl selected from (CH₃)₂FC—, CF₃CH₂—, CH₃CF₂—, CF₃—, CH₂F—, and CHF₂—.

In one particularly preferred embodiment, the invention provides a compound of formula (I), or a solvate, or a pharmaceutically acceptable salt thereof, wherein R⁶ is CF₃—.

In one preferred embodiment, the invention provides a compound of formula (I), or a solvate, or a pharmaceutically acceptable salt thereof, wherein R⁶ is (CH₃)₂FC—.

In one preferred embodiment, the invention provides a compound of formula (I), or a solvate, or a pharmaceutically acceptable salt thereof, wherein R⁶ is CF₃CH₂—.

In one preferred embodiment, the invention provides a compound of formula (I), or a solvate, or a pharmaceutically acceptable salt thereof, wherein R⁶ is CH₃CF₂—.

In one preferred embodiment, the invention provides a compound of formula (I), or a solvate, or a pharmaceutically acceptable salt thereof, wherein R⁶ is CH₂F—.

In one preferred embodiment, the invention provides a compound of formula (I), or a solvate, or a pharmaceutically acceptable salt thereof, wherein R⁶ is CH₃CF₂—.

In one preferred embodiment, the invention provides a compound of formula (I), or a solvate, or a pharmaceutically acceptable salt thereof, wherein R⁶ is CH₃CF₂— with a structure selected from:

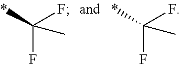

In one preferred embodiment, the invention provides a compound of formula (I), or a solvate, or a pharmaceutically acceptable salt thereof, wherein R⁶ is CH₃CF₂— with the structure:

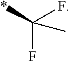

In one preferred embodiment, the invention provides a compound of formula (I), or a solvate, or a pharmaceutically acceptable salt thereof, wherein R⁶ is CH₃CF₂— with the structure:

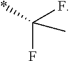

In one particularly preferred embodiment, the invention provides a compound of formula (I), or a solvate, or a pharmaceutically acceptable salt thereof, wherein R⁶ is CF₃— selected from the structures:

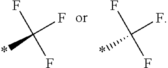

In one particularly preferred embodiment, the invention provides a compound of formula (I), or a solvate, or a pharmaceutically acceptable salt thereof, wherein R⁶ is CF₃— having the structure:

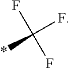

In one particularly preferred embodiment, the invention provides a compound of formula (I), or a solvate, or a pharmaceutically acceptable salt thereof, wherein R⁶ is CF₃— having the structure:

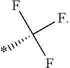

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is phenyl as described herein.

In one more preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is phenyl as described herein.

In one more preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is a saturated monocyclic 3-6 membered cycloalkyl as described herein.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is a C₁₋₆alkyl as described herein.

In one preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is a 4-6 membered heterocycloalkyl as described herein.

In one particularly preferred embodiment, the invention provides a compound of formula (I), or a solvate, or a pharmaceutically acceptable salt thereof, wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy, R² is H, R³ is H, R⁴ and R⁵ are both H, and R⁶ is a haloC₁₋₆alkyl as described herein.

In one preferred embodiment, the invention provides a compound of formula (III), or a solvate, or a pharmaceutically acceptable salt thereof:

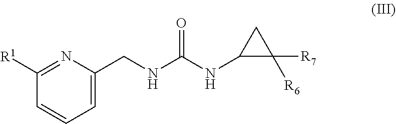

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one particularly preferred embodiment, the invention provides a compound of formula (IV), or a solvate, or a pharmaceutically acceptable salt thereof:

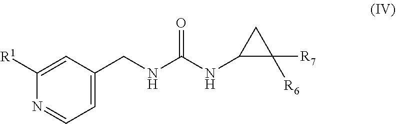

wherein R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one particularly preferred embodiment, the invention provides a compound of formula (V), or a solvate, or a pharmaceutically acceptable salt thereof:

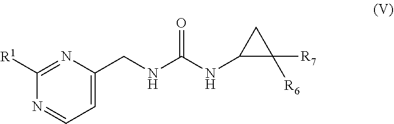

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (I), or a solvate, or a pharmaceutically acceptable salt thereof, wherein R⁷ is H or C₁₋₆alkyl.

In one more preferred embodiment, the invention provides a compound of formula (I), or a solvate, or a pharmaceutically acceptable salt thereof, wherein R⁷ is H.

In one preferred embodiment, the invention provides a compound of formula (I), or a solvate, or a pharmaceutically acceptable salt thereof, wherein R⁷ is C₁₋₆alkyl.

In one preferred embodiment, the invention provides a compound of formula (I), or a solvate, or a pharmaceutically acceptable salt thereof, wherein R⁷ is C₁₋₆alkyl selected from CH₃—, CH₃CH₂—, CH₃CH₂CH₂—, (CH₃)₂CH₂—, (CH₃CH₂CH₂CH₂—), (CH₃)₂CH₂CH₂—, CH₃CH(CH₃)CH₂—, and (CH₃)₃C—.

In one preferred embodiment, the invention provides a compound of formula (I), or a solvate, or a pharmaceutically acceptable salt thereof, wherein R⁷ is CH₃—.

In one preferred embodiment, the invention provides a compound of formula (III), or a solvate, or a pharmaceutically acceptable salt thereof:

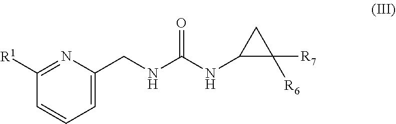

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (III), or a solvate, or a pharmaceutically acceptable salt thereof:

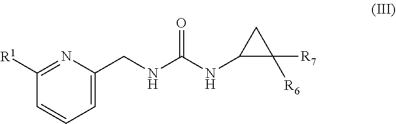

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is selected from H, and C₁₋₆alkyl.

In one more preferred embodiment, the invention provides a compound of formula (III), or a solvate, or a pharmaceutically acceptable salt thereof:

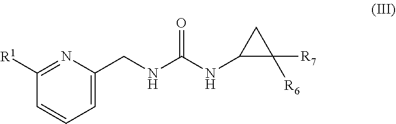

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is H.

In one preferred embodiment, the invention provides a compound of formula (III), or a solvate, or a pharmaceutically acceptable salt thereof:

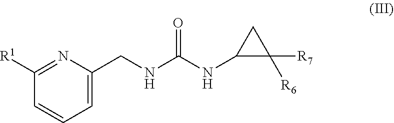

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is C₁₋₆alkyl.

In one more preferred embodiment, the invention provides a compound of formula (III), or a solvate, or a pharmaceutically acceptable salt thereof:

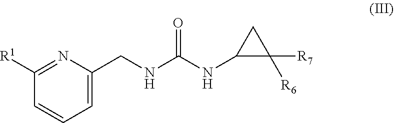

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one moe preferred embodiment, the invention provides a compound of formula (III), or a solvate, or a pharmaceutically acceptable salt thereof:

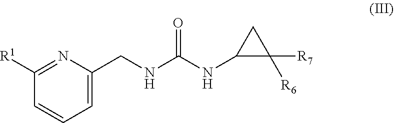

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, and C₁₋₆alkyl.

In one more preferred embodiment, the invention provides a compound of formula (III), or a solvate, or a pharmaceutically acceptable salt thereof:

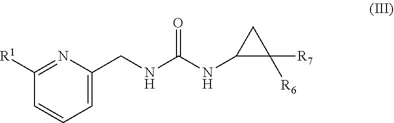

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is H.

In one more preferred embodiment, the invention provides a compound of formula (III), or a solvate, or a pharmaceutically acceptable salt thereof:

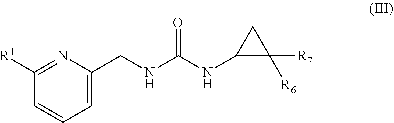

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylalkoxy, 4-6 membered heterocycloalkyl, 4-6 membered heterocycloalkylC0-6alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is C₁₋₆alkyl.

In one preferred embodiment, the invention provides a compound of formula (III), or a solvate, or a pharmaceutically acceptable salt thereof:

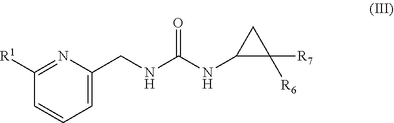

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ a C₁₋₆alkyl, and
R⁷ is selected from H, C₁₋₆alkyl, haloC₁₋₆alkyl, and halogen.

In one preferred embodiment, the invention provides a compound of formula (III), or a solvate, or a pharmaceutically acceptable salt thereof:

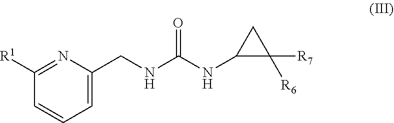

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is C₁₋₆alkyl, and
R⁷ is selected from H, and C₁₋₆alkyl.

In one preferred embodiment, the invention provides a compound of formula (III), or a solvate, or a pharmaceutically acceptable salt thereof:

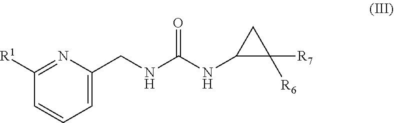

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is C₁₋₆alkyl, and
R⁷ is H.

In one preferred embodiment, the invention provides a compound of formula (III), or a solvate, or a pharmaceutically acceptable salt thereof:

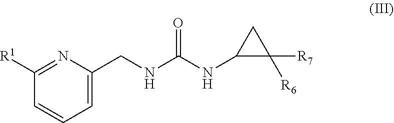

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is C₁₋₆alkyl, and
R⁷ is C₁₋₆alkyl.

In one preferred embodiment, the invention provides a compound of formula (III), or a solvate, or a pharmaceutically acceptable salt thereof:

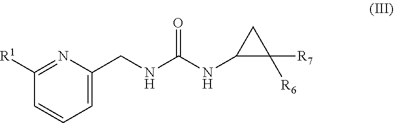

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, C₁₋₆alkyl, haloC₁₋₆alkyl, and halogen.

In one preferred embodiment, the invention provides a compound of formula (III), or a solvate, or a pharmaceutically acceptable salt thereof:

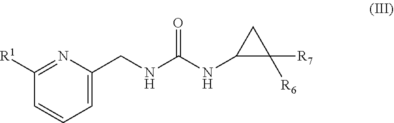

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, and C₁₋₆alkyl.

In one preferred embodiment, the invention provides a compound of formula (III), or a solvate, or a pharmaceutically acceptable salt thereof:

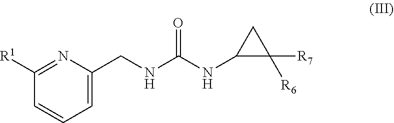

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is H.

In one preferred embodiment, the invention provides a compound of formula (III), or a solvate, or a pharmaceutically acceptable salt thereof:

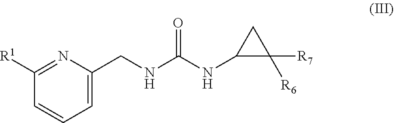

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is C₁₋₆alkyl.

In one preferred embodiment, the invention provides a compound of formula (III), or a solvate, or a pharmaceutically acceptable salt thereof:

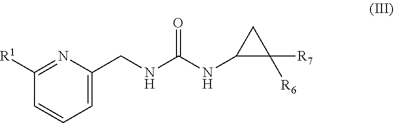

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a haloC₁₋₆alkyl as described herein, and
R⁷ is selected from H, C₁₋₆alkyl, haloC₁₋₆alkyl, and halogen.

In one more preferred embodiment, the invention provides a compound of formula (III), or a solvate, or a pharmaceutically acceptable salt thereof:

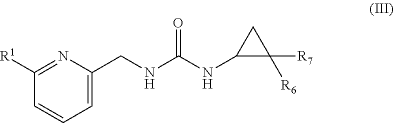

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a haloC₁₋₆alkyl as described herein, and
R⁷ is selected from H, and C₁₋₆alkyl.

In one more preferred embodiment, the invention provides a compound of formula (III), or a solvate, or a pharmaceutically acceptable salt thereof:

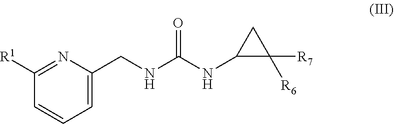

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a haloC₁₋₆alkyl as described herein, and
R⁷ is H.

In one more preferred embodiment, the invention provides a compound of formula (III), or a solvate, or a pharmaceutically acceptable salt thereof:

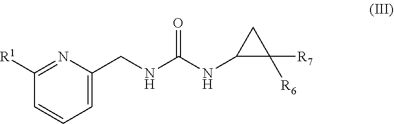

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a haloC₁₋₆alkyl as described herein, and
R⁷ is C₁₋₆alkyl.

In one preferred embodiment, the invention provides a compound of formula (IV), or a solvate, or a pharmaceutically acceptable salt thereof:

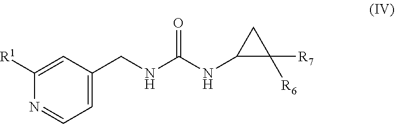

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (IV), or a solvate, or a pharmaceutically acceptable salt thereof:

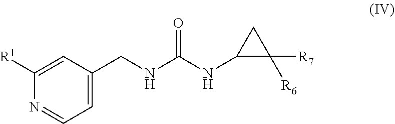

wherein
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is selected from H, and C₁₋₆alkyl.

In one preferred embodiment, the invention provides a compound of formula (IV), or a solvate, or a pharmaceutically acceptable salt thereof:

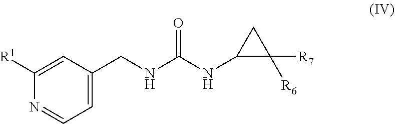

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is H.

In one preferred embodiment, the invention provides a compound of formula (IV), or a solvate, or a pharmaceutically acceptable salt thereof:

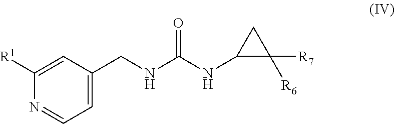

wherein
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is C₁₋₆alkyl.

In one preferred embodiment, the invention provides a compound of formula (IV), or a solvate, or a pharmaceutically acceptable salt thereof:

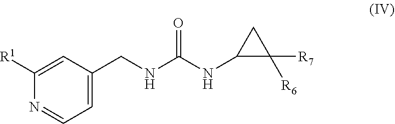

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (IV), or a solvate, or a pharmaceutically acceptable salt thereof:

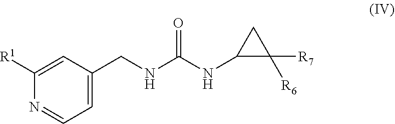

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, and C₁₋₆alkyl.

In one preferred embodiment, the invention provides a compound of formula (IV), or a solvate, or a pharmaceutically acceptable salt thereof:

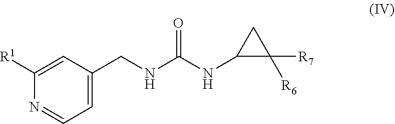

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is H.

In one preferred embodiment, the invention provides a compound of formula (IV), or a solvate, or a pharmaceutically acceptable salt thereof:

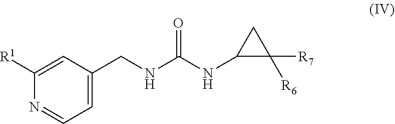

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is C₁₋₆alkyl.

In one preferred embodiment, the invention provides a compound of formula (IV), or a solvate, or a pharmaceutically acceptable salt thereof:

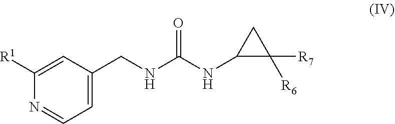

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ a C₁₋₆alkyl, and
R⁷ is selected from H, C₁₋₆alkyl, haloC₁₋₆alkyl, and halogen.

In one preferred embodiment, the invention provides a compound of formula (IV), or a solvate, or a pharmaceutically acceptable salt thereof:

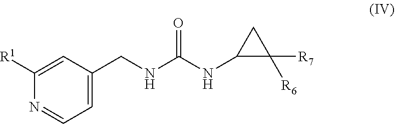

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is selected from H, and C₁₋₆alkyl.

In one preferred embodiment, the invention provides a compound of formula (IV), or a solvate, or a pharmaceutically acceptable salt thereof:

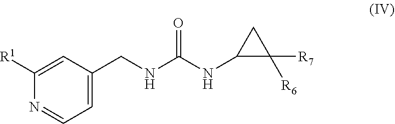

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is H.

In one preferred embodiment, the invention provides a compound of formula (IV), or a solvate, or a pharmaceutically acceptable salt thereof:

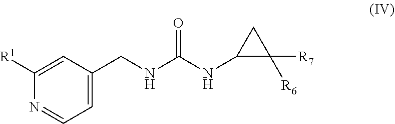

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is C₁₋₆alkyl.

In one preferred embodiment, the invention provides a compound of formula (IV), or a solvate, or a pharmaceutically acceptable salt thereof:

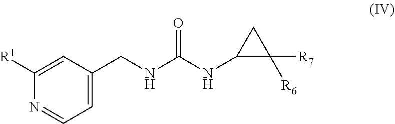

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, C₁₋₆alkyl, haloC₁₋₆alkyl, and halogen.

In one preferred embodiment, the invention provides a compound of formula (IV), or a solvate, or a pharmaceutically acceptable salt thereof:

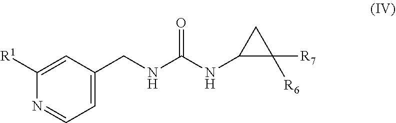

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, and C₁₋₆alkyl.

In one preferred embodiment, the invention provides a compound of formula (IV), or a solvate, or a pharmaceutically acceptable salt thereof:

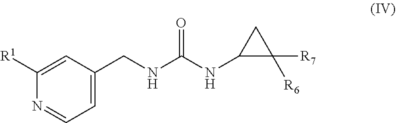

wherein
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is H.

In one preferred embodiment, the invention provides a compound of formula (IV), or a solvate, or a pharmaceutically acceptable salt thereof:

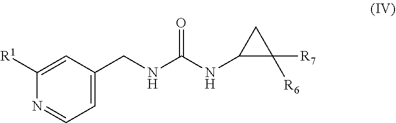

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is C₁₋₆alkyl.

In one preferred embodiment, the invention provides a compound of formula (IV), or a solvate, or a pharmaceutically acceptable salt thereof:

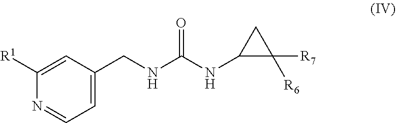

wherein
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is selected from H, C₁₋₆alkyl, haloC₁₋₆alkyl, and halogen.

In one preferred embodiment, the invention provides a compound of formula (IV), or a solvate, or a pharmaceutically acceptable salt thereof:

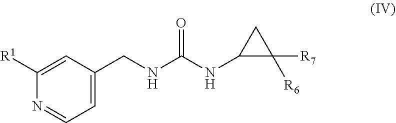

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is selected from H, and C₁₋₆alkyl.

In one particularly preferred embodiment, the invention provides a compound of formula (IV), or a solvate, or a pharmaceutically acceptable salt thereof:

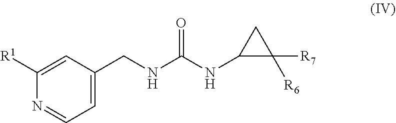

wherein
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is H.

In one particularly preferred embodiment, the invention provides a compound of formula (IV), or a solvate, or a pharmaceutically acceptable salt thereof:

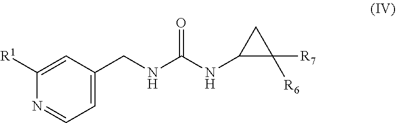

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is C₁₋₆alkyl.

In one preferred embodiment, the invention provides a compound of formula (V), or a solvate, or a pharmaceutically acceptable salt thereof:

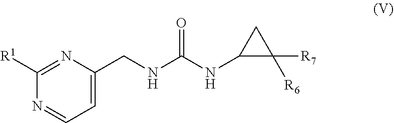

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (V), or a solvate, or a pharmaceutically acceptable salt thereof:

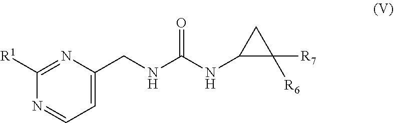

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is selected from H, and C₁₋₆alkyl.

In one preferred embodiment, the invention provides a compound of formula (V), or a solvate, or a pharmaceutically acceptable salt thereof:

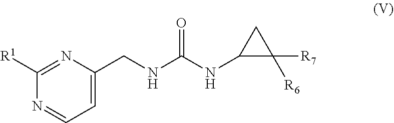

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is H.

In one preferred embodiment, the invention provides a compound of formula (V), or a solvate, or a pharmaceutically acceptable salt thereof:

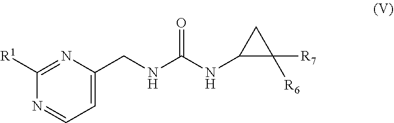

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is phenyl, which is optionally substituted with one, two, or three substituents independently selected from halogen, haloC₁₋₆alkyl, and haloC₁₋₆alkoxy, and
R⁷ is C₁₋₆alkyl.

In one preferred embodiment, the invention provides a compound of formula (V), or a solvate, or a pharmaceutically acceptable salt thereof:

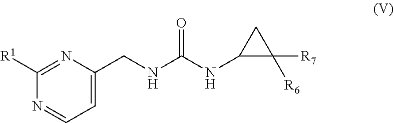

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, C₁₋₆alkyl, haloC₁₋₆alkyl, and halogen.

In one preferred embodiment, the invention provides a compound of formula (V), or a solvate, or a pharmaceutically acceptable salt thereof:

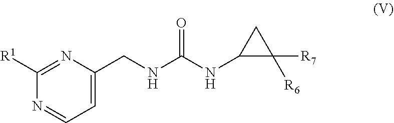

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is selected from H, and C₁₋₆alkyl.

In one preferred embodiment, the invention provides a compound of formula (V), or a solvate, or a pharmaceutically acceptable salt thereof:

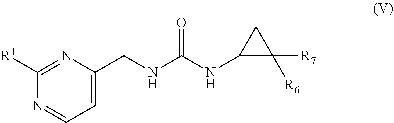

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R⁷ is H.

In one preferred embodiment, the invention provides a compound of formula (V), or a solvate, or a pharmaceutically acceptable salt thereof:

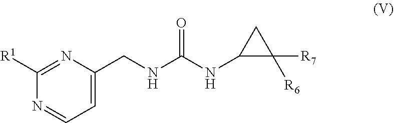

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a saturated monocyclic 3-6 membered cycloalkyl as described herein, which is optionally substituted with one, two, or three substituents independently selected from halogen, haloC₁₋₆alkyl, and haloC₁₋₆alkoxy, and
R¹ is C₁₋₆alkyl.

In one preferred embodiment, the invention provides a compound of formula (V), or a solvate, or a pharmaceutically acceptable salt thereof:

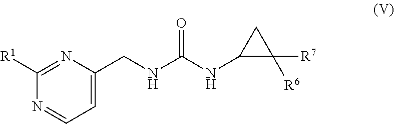

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ a C₁₋₆alkyl, and
R⁷ is selected from H, C₁₋₆alkyl, haloC₁₋₆alkyl, and halogen.

In one preferred embodiment, the invention provides a compound of formula (V), or a solvate, or a pharmaceutically acceptable salt thereof:

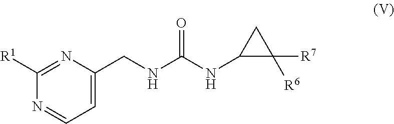

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is selected from H, and C₁₋₆alkyl.

In one preferred embodiment, the invention provides a compound of formula (V), or a solvate, or a pharmaceutically acceptable salt thereof:

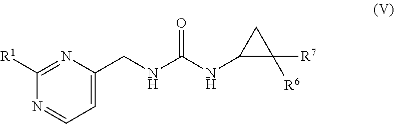

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is H.

In one preferred embodiment, the invention provides a compound of formula (V), or a solvate, or a pharmaceutically acceptable salt thereof:

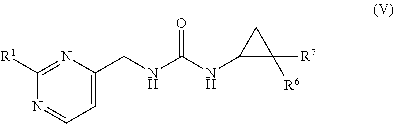

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a C₁₋₆alkyl, and
R⁷ is C₁₋₆alkyl.

In one preferred embodiment, the invention provides a compound of formula (V), or a solvate, or a pharmaceutically acceptable salt thereof:

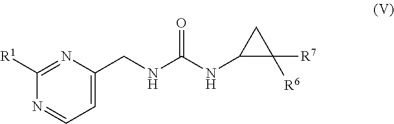

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl,
heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, C₁₋₆alkyl, haloC₁₋₆alkyl, and halogen.

In one preferred embodiment, the invention provides a compound of formula (V), or a solvate, or a pharmaceutically acceptable salt thereof:

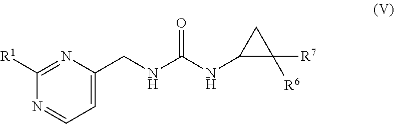

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is selected from H, and C₁₋₆alkyl.

In one preferred embodiment, the invention provides a compound of formula (V), or a solvate, or a pharmaceutically acceptable salt thereof:

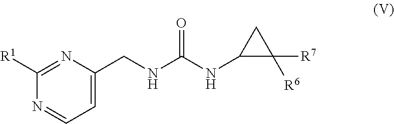

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is H.

In one preferred embodiment, the invention provides a compound of formula (V), or a solvate, or a pharmaceutically acceptable salt thereof:

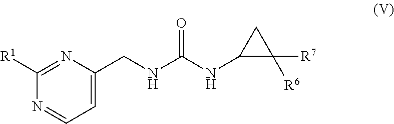

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is a 4-6 membered heterocycloalkyl as described herein, and
R⁷ is C₁₋₆alkyl.

In one preferred embodiment, the invention provides a compound of formula (V), or a solvate, or a pharmaceutically acceptable salt thereof:

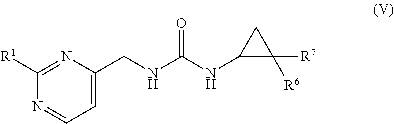

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is selected from H, C₁₋₆alkyl, haloC₁₋₆alkyl, and halogen.

In one preferred embodiment, the invention provides a compound of formula (V), or a solvate, or a pharmaceutically acceptable salt thereof:

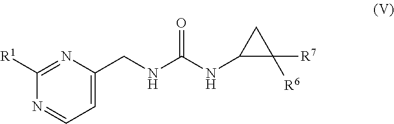

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is selected from H, and C₁₋₆alkyl.

In one preferred embodiment, the invention provides a compound of formula (V), or a solvate, or a pharmaceutically acceptable salt thereof:

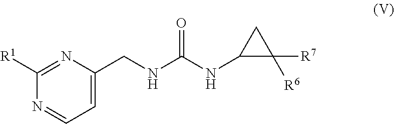

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is H.

In one preferred embodiment, the invention provides a compound of formula (V), or a solvate, or a pharmaceutically acceptable salt thereof:

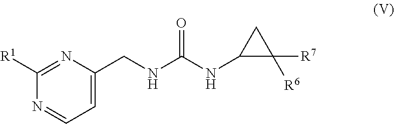

wherein
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is haloC₁₋₆alkyl as described herein, and
R⁷ is C₁₋₆alkyl.

In one preferred embodiment, the invention provides a compound of formula (I), or a solvate, or a pharmaceutically acceptable salt thereof, wherein R¹ is cyano, halogen, C₁₋₆alkoxy, haloC₁₋₆alkoxy, 5 membered heteroaryl, 4-6 membered heterocycloalkylC₀₋₆alkoxy, haloC₁₋₆alkyl, 3-6 membered cycloalkylC₀₋₆alkoxy, and phenoxy, all as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is cyano, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is halogen as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is C₁₋₆alkoxy as described herein, R² is H, R³ is H, R⁴ and R⁵ are both H, R⁶ is unsubstituted phenyl, and R⁷ is H.

In one more preferred embodiment, the invention provides a compound of formula (I), or a solvate, or a pharmaceutically acceptable salt thereof, wherein R¹ is haloC₁₋₆alkoxy as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is 5 membered heteroaryl as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is 4-6 membered heterocycloalkylC₀₋₆alkoxy as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is a 3-6 membered cycloalkylC₀₋₆alkoxy as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is phenoxy as described herein, R² is H, R³ is H, R⁴ and R⁵ are both H, R⁶ is unsubstituted phenyl, and R⁷ is H.

In one preferred embodiment, the invention provides a compound of formula (I), or a solvate, or a pharmaceutically acceptable salt thereof, wherein R¹ is cyano, halogen, C₁₋₆alkoxy, haloC₁₋₆alkoxy, 5 membered heteroaryl, 4-6 membered heterocycloalkylC₀₋₆alkoxy, haloC₁₋₆alkyl, 3-6 membered cycloalkylC₀₋₆alkoxy, and phenoxy, all as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is cyano, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is halogen as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is C₁₋₆alkoxy as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is haloC₁₋₆alkoxy as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is a 5 membered heteroaryl as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is a 4-6 membered heterocycloalkylC₀₋₆alkoxy as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is a 3-6 membered cycloalkylC₀₋₆alkoxy as described herein, R² is H, R³ is H, R⁴ and R⁵ are both H, R⁶ is a unsubstituted saturated monocyclic 3-6 membered cycloalkyl as described herein, and R⁷ is H.

In one preferred embodiment, the invention provides a compound of formula (I), or a solvate, or a pharmaceutically acceptable salt thereof, wherein R¹ is phenoxy as described herein, R² is H, R³ is H, R⁴ and R⁵ are both H, R⁶ is a unsubstituted saturated monocyclic 3-6 membered cycloalkyl as described herein, and R⁷ is H.

In one embodiment, the invention provides a compound of formula (I), or a solvate, or a pharmaceutically acceptable salt thereof, wherein R¹ is cyano, halogen, C₁₋₆alkoxy, haloC₁₋₆alkoxy, 5 membered heteroaryl, 4-6 membered heterocycloalkylC₀₋₆alkoxy, haloC₁₋₆alkyl, 3-6 membered cycloalkylC₀₋₆alkoxy, and phenoxy, all as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one preferred embodiment, the invention provides a compound of formula (I), or a solvate, or a pharmaceutically acceptable salt thereof, wherein R¹ is cyano, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one embodiment, the invention provides a compound of formula (I), or a solvate, or a pharmaceutically acceptable salt thereof, wherein R¹ is halogen as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one embodiment, the invention provides a compound of formula (I), or a solvate, or a pharmaceutically acceptable salt thereof, wherein R¹ is C₁₋₆alkoxy as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one more preferred embodiment, the invention provides a compound of formula (I), or a solvate, or a pharmaceutically acceptable salt thereof, wherein R¹ is haloC₁₋₆alkoxy as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one preferred embodiment, the invention provides a compound of formula (I), or a solvate, or a pharmaceutically acceptable salt thereof, wherein R¹ is a 5 membered heteroaryl as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one embodiment, the invention provides a compound of formula (I), or a solvate, or a pharmaceutically acceptable salt thereof, wherein R¹ is a 4-6 membered heterocycloalkylC₀₋₆alkoxy as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one preferred embodiment, the invention provides a compound of formula (I), or a solvate, or a pharmaceutically acceptable salt thereof, wherein R¹ is a 3-6 membered cycloalkylC₀₋₆alkoxy as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In one embodiment, the invention provides a compound of formula (I), or a solvate, or a pharmaceutically acceptable salt thereof, wherein R¹ is phenoxy as described herein, R² is H, R³ is H, R⁴ and R⁵ are both H, R⁶ is a unsubstituted saturated monocyclic 3-6 membered cycloalkyl selected from cyclopropyl, cyclobutyl, cyclopentyl and cyclohexyl as described herein, and R⁷ is H.

In some embodiment described herein the unsubstituted saturated monocyclic 3-6 membered cycloalkyl is unsubstituted cyclohexyl.

In one preferred embodiment, the invention provides a compound of formula (I), or a solvate, or a pharmaceutically acceptable salt thereof, wherein R¹ is cyano, halogen, C₁₋₆alkoxy, haloC₁₋₆alkoxy, 5 membered heteroaryl, 4-6 membered heterocycloalkylC₀₋₆alkoxy, haloC₁₋₆alkyl, 3-6 membered cycloalkylC₀₋₆alkoxy, and phenoxy, all as described herein, R² is H, R³ is H, R⁴ and R⁵ are both H, R⁶ is haloC₁₋₆alkyl as described herein, and R⁷ is H.

In one preferred embodiment, the invention provides a compound of formula (I), or a solvate, or a pharmaceutically acceptable salt thereof, wherein R¹ is cyano, R² is H, R³ is H, R⁴ and R⁵ are both H, R⁶ is haloC₁₋₆alkyl as described herein, and R⁷ is H.

In one preferred embodiment, the invention provides a compound of formula (I), or a solvate, or a pharmaceutically acceptable salt thereof, wherein R¹ is halogen as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is C₁₋₆alkoxy as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is haloC₁₋₆alkoxy as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is 5 membered heteroaryl, as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is 4-6 membered heterocycloalkylC₀₋₆alkoxy as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is a 3-6 membered cycloalkylalkoxy as described herein, R² is H, R³ is H, R⁴ and R⁵ are both H, R⁶ is haloC₁₋₆alkyl as described herein, and R⁷ is H.

In one more preferred embodiment, the invention provides a compound of formula (I), or a solvate, or a pharmaceutically acceptable salt thereof, wherein R¹ is phenoxy as described herein, R² is H, R³ is H, R⁴ and R⁵ are both H, R⁶ is haloC₁₋₆alkyl as described herein, and R⁷ is H.

In some of the aforementioned embodiments, R⁶ is haloC₁₋₆alkyl selected from (CH₃)₂FC—, CF₃CH₂—, CH₃CF₂—, CF₃—, CH₂F—, and CHF₂—.

In one preferred embodiment, the invention provides a compound of formula (I), or a solvate, or a pharmaceutically acceptable salt thereof, wherein R⁶ is CH₃CF₂—.

In one particularly preferred embodiment, the invention provides a compound of formula (I), or a solvate, or a pharmaceutically acceptable salt thereof, wherein R⁶ is CH₃CF₂— with a structure selected from:

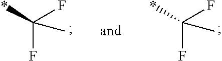

In one particularly preferred embodiment, the invention provides a compound of formula (I), or a solvate, or a pharmaceutically acceptable salt thereof, wherein R⁶ is CH₃CF₂— with the structure:

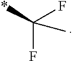

In one particularly preferred embodiment, the invention provides a compound of formula (I), or a solvate, or a pharmaceutically acceptable salt thereof, wherein R⁶ is CH₃CF₂— with a structure:

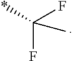

In one particularly preferred embodiment, the invention provides a compound of formula (I), or a solvate, or a pharmaceutically acceptable salt thereof, wherein R⁶ is CF₃—.

In one particularly preferred embodiment, the invention provides a compound of formula (I), or a solvate, or a pharmaceutically acceptable salt thereof, wherein R⁶ is CF₃— having the structures:

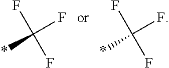

In one particularly preferred embodiment, the invention provides a compound of formula (I), or a solvate, or a pharmaceutically acceptable salt thereof, wherein R⁶ is CF₃— having the structure:

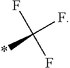

In one particularly preferred embodiment, the invention provides a compound of formula (I), or a solvate, or a pharmaceutically acceptable salt thereof, wherein R⁶ is CF₃— having the structure:

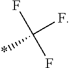

In one embodiment, the invention provides a compound of formula (I), or a solvate, or a pharmaceutically acceptable salt thereof, wherein R¹ is cyano, halogen, C₁₋₆alkoxy, haloC₁₋₆alkoxy, 5 membered heteroaryl, 4-6 membered heterocycloalkylC₀₋₆alkoxy, haloC₁₋₆alkyl, 3-6 membered cycloalkylC₀₋₆alkoxy, and phenoxy, all as described herein, R² is H, R³ is H, R⁴ and R⁵ are both H, R⁶ is haloC₁₋₆alkoxy as described herein, and R⁷ is H.

In one preferred embodiment, the invention provides a compound of formula (I), or a solvate, or a pharmaceutically acceptable salt thereof, wherein R¹ is cyano, R² is H, R³ is H, R⁴ and R⁵ are both H, R⁶ is haloC₁₋₆alkoxy as described herein, and R⁷ is H.

In one embodiment, the invention provides a compound of formula (I), or a solvate, or a pharmaceutically acceptable salt thereof, wherein R¹ is halogen as described herein, R² is H, R³ is H, R⁴ and R⁵ are both H, R⁶ is haloC₁₋₆alkoxy as described herein, and R⁷ is H.

In one embodiment, the invention provides a compound of formula (I), or a solvate, or a pharmaceutically acceptable salt thereof, wherein R¹ is C₁₋₆alkoxy as described herein, R² is H, R³ is H, R⁴ and R⁵ are both H, R⁶ is haloC₁₋₆alkoxy as described herein, and R⁷ is H.

In one preferred embodiment, the invention provides a compound of formula (I), or a solvate, or a pharmaceutically acceptable salt thereof, wherein R¹ is haloC₁₋₆alkoxy as described herein, R² is H, R³ is H, R⁴ and R⁵ are both H, R⁶ is haloC₁₋₆alkoxy as described herein, and R⁷ is H.

In one preferred embodiment, the invention provides a compound of formula (I), or a solvate, or a pharmaceutically acceptable salt thereof, wherein R¹ is 5 membered heteroaryl, as described herein, R² is H, R³ is H, R⁴ and R⁵ are both H, R⁶ is haloC₁₋₆alkoxy as described herein, and R⁷ is H.

In one embodiment, the invention provides a compound of formula (I), or a solvate, or a pharmaceutically acceptable salt thereof, wherein R¹ is 4-6 membered heterocycloalkylC₀₋₆alkoxy as described herein, R² is H, R³ is H, R⁴ and R⁵ are both H, R⁶ is haloC₁₋₆alkoxy as described herein, and R⁷ is H.

In one preferred embodiment, the invention provides a compound of formula (I), or a solvate, or a pharmaceutically acceptable salt thereof, wherein R¹ is a 3-6 membered cycloalkylC₀₋₆alkoxy as described herein, R² is H, R³ is H, R⁴ and R⁵ are both H, R⁶ is haloC₁₋₆alkoxy as described herein, and R⁷ is H.

In one embodiment, the invention provides a compound of formula (I), or a solvate, or a pharmaceutically acceptable salt thereof, wherein R¹ is phenoxy as described herein, R² is H, R³ is H, R⁴ and R⁵ are both H, R⁶ is haloC₁₋₆alkoxy as described herein, and R⁷ is H.

In some of the aforementioned embodiments, R⁶ is haloC₁₋₆alkoxy is selected from CHF₂O—, and CH₂FO—, CF₃O—, FCH₂CFHCH₂O—, CH₂FCH₂O—, and CF₃CH₂O—.

In one more preferred embodiment, the invention provides a compound of formula (VI), or a solvate, or a pharmaceutically acceptable salt thereof, wherein

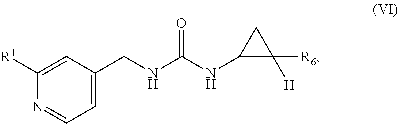

wherein R¹ and R⁶ are as described herein.

In one more preferred embodiment, the invention provides a compound of formula (VII), or a solvate, or a pharmaceutically acceptable salt thereof, wherein

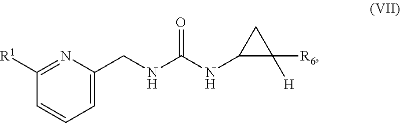

wherein R¹ and R⁶ are as described herein.

In one more preferred embodiment, the invention provides a compound of formula (VIII), or a solvate, or a pharmaceutically acceptable salt thereof, wherein

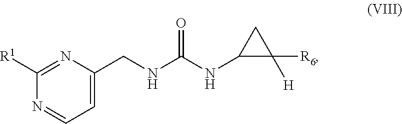

wherein R¹ and R⁶ are as described herein.

Exemplary formula (I) compounds in Table 1 were made, characterized, and tested for activation of Kv7.2 (EC₅₀ less than 10 micromolar, μM) and Kv7.2 activation max % according to the methods of this invention, and have the following structures and corresponding names (OpenEye Lexichem, Version 1.2.0, OpenEye Scientific Software, Santa Fe, NM, USA; https://www.eyesopen.com/lexichem-tk).

In some embodiments, the compound is selected from Table 1, or a solvate or a pharmaceutically acceptable salt thereof.

TABLE 1

| | | | Kv7.2 | Kv7.2 |
|---|---|---|---|---|
| | | | EC₅₀ | Emax |
| Example | Name | Structure | (μM) | (%) |
| 1 | 1-[(4-cyanopyridin-2- yl)methyl]-3-[(1R,2S)- 2- phenylcyclopropyl]urea | 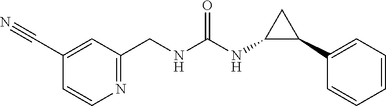 [SMILES: N#Cc1ccnc(CNC(=O)N[C@@H]2C[C@H]2c2ccccc2)c1] | 4.68 | 73 |
| 2 | 1-[rac-(1R,2S)-2- cyclohexylcyclopropyl]- 3-[(2-pyrazol-1- ylpyridin-4- yl)methyl]urea | 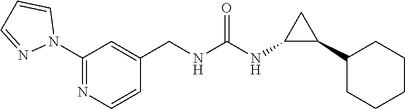 [SMILES: O=C(NCc1ccnc(-n2cccn2)c1)N[C@@H]1C[C@H]1C1CCCCC1] | 0.57 | 84 |
| 3 | 1-[(6-chloropyridin-2- yl)methyl]-3-[rac- (1R,2S)-2- cyclohexylcyclopropyl] urea | 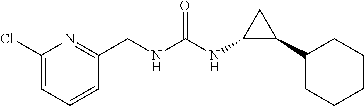 [SMILES: O=C(NCc1cccc(Cl)n1)N[C@@H]1C[C@H]1C1CCCCC1] | 1.54 | 77 |
| 4 | 1-[(2-propan-2- ylpyrimidin-4- yl)methyl]-3-[rac- (1R,2S)-2- cyclohexylcyclopropyl] urea | 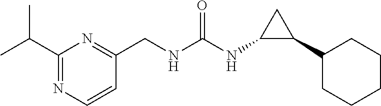 [SMILES: CC(C)c1nccc(CNC(=O)N[C@@H]2C[C@H]2C2CCCCC2)n1] | 4.40 | 71 |
| 5 | 1-[(2-methoxypyridin- 4-yl)methyl]-3-[rac- (1R,2S)-2- phenylcyclopropyl]urea | 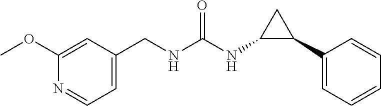 [SMILES: COc1cc(CNC(=O)N[C@@H]2C[C@H]2c2ccccc2)ccn1] | 3.75 | 44 |
| 6 | 1-[(1R,2S)-2- phenylcyclopropyl]-3- [(2-propan-2- yloxypyridin-4- yl)methyl]urea | 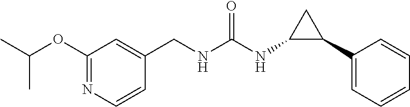 [SMILES: CC(C)Oc1cc(CNC(=O)N[C@@H]2C[C@H]2c2ccccc2)ccn1] | 0.66 | 64 |
| 7 | 1-[[2- (difluoromethoxy) pyridin-4-yl]methyl]-3- [(1R,2S)-2- phenylcyclopropyl]urea | 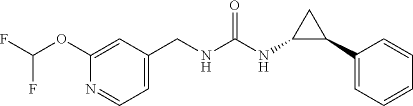 [SMILES: O=C(NCc1ccnc(OC(F)F)c1)N[C@@H]1C[C@H]1c1ccccc1] | 0.10 | 71 |
| 8 | 1-[(2-cyanopyridin-4- yl)methyl]-3-[rac- (1R,2S)-2- phenylcyclopropyl]urea | 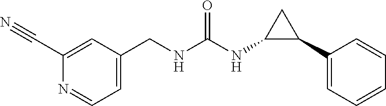 [SMILES: N#Cc1cc(CNC(=O)N[C@@H]2C[C@H]2c2ccccc2)ccn1] | 4.18 | 40 |
| 9 | 1-[(2-methoxypyridin- 4-yl)methyl]-3- [(1R,2S)-2- phenylcyclopropyl]urea | 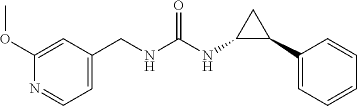 [SMILES: COc1cc(CNC(=O)N[C@@H]2C[C@H]2c2ccccc2)ccn1] | 3.41 | 78 |
| 10 | 1-[(2-cyanopyridin-4- yl)methyl]-3-[(1R,2S)- 2- phenylcyclopropyl]urea | 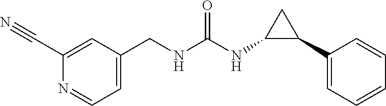 [SMILES: N#Cc1cc(CNC(=O)N[C@@H]2C[C@H]2c2ccccc2)ccn1] | 1.19 | 43 |
| 11 | 1-(2- cyclohexylcyclopropyl)- 3-[(2-imidazol-1- ylpyridin-4- yl)methyl]urea | 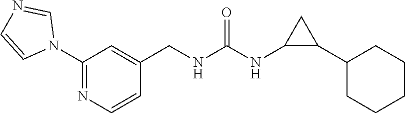 [SMILES: O=C(NCc1ccnc(-n2ccnc2)c1)NC1CC1C1CCCCC1] | 2.14 | 52 |
| 12 | 1-[(2-pyrazol-1- ylpyridin-4-yl)methyl]- 3-[rac-(1R,2S)-2- phenylcyclopropyl]urea | 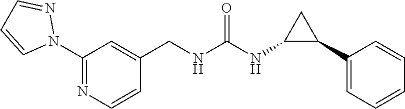 [SMILES: O=C(NCc1ccnc(-n2cccn2)c1)N[C@@H]1C[C@H]1c1ccccc1] | 0.67 | 69 |
| 13 | 1-[(1R,2S)-2- phenylcyclopropyl]-3- [(2-pyrazol-1-ylpyridin- 4-yl)methyl]urea | 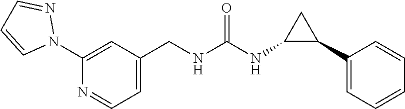 [SMILES: O=C(NCc1ccnc(-n2cccn2)c1)N[C@@H]1C[C@H]1c1ccccc1] | 1.34 | 74 |
| 14 | 1-[(1R,2S)-2- phenylcyclopropyl]-3- [[2-(2,2,2- trifluoroethoxy)pyridin- 4-yl]methyl]urea | 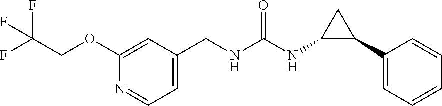 [SMILES: O=C(NCc1ccnc(OCC(F)(F)F)c1)N[C@@H]1C[C@H]1c1ccccc1] | 0.07 | 71 |
| 15 | 1-[(1S,2R)-2- cyclohexylcyclopropyl]- 3-[(2-imidazol-1- ylpyridin-4- yl)methyl]urea | 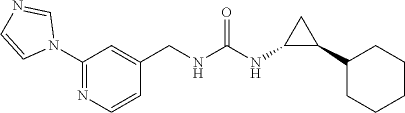 [SMILES: O=C(NCc1ccnc(-n2ccnc2)c1)N[C@@H]1C[C@H]1C1CCCCC1] | 1.57 | 56 |
| 16 | 1-[2-(oxetan-3- yloxy)pyridin-4- yl]methyl]-3-[(1R,2S)- 2- phenylcyclopropyl]urea | 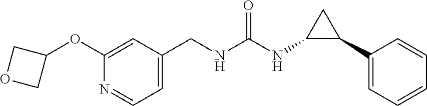 [SMILES: O=C(NCc1ccnc(OC2COC2)c1)N[C@@H]1C[C@H]1c1ccccc1] | 0.96 | 57 |
| 17 | 1-[(2-chloropyridin-4- yl)methyl]-3-[rac- (1R,2S)-2- cyclohexylcyclopropyl] urea | 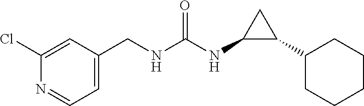 [SMILES: O=C(NCc1ccnc(Cl)c1)N[C@H]1C[C@@H]1C1CCCCC1] | 1.76 | 61 |
| 18 | 1-[(6-cyanopyridin-2- yl)methyl]-3-[(1R,2S)- 2- phenylcyclopropyl]urea | 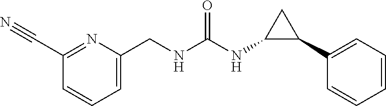 [SMILES: N#Cc1cccc(CNC(=O)N[C@@H]2C[C@H]2c2ccccc2)n1] | 3.02 | 62 |
| 19 | 1-[(2-chloropyridin-4- yl)methyl]-3-[(1R,2S)- 2- phenylcyclopropyl]urea | 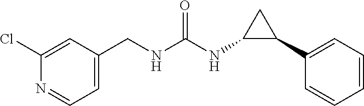 [SMILES: O=C(NCc1ccnc(Cl)c1)N[C@@H]1C[C@H]1c1ccccc1] | 6.83 | 60 |
| 20 | 1-[[2-(oxan-4- yloxy)pyridin-4- yl]methyl]-3-[(1R,2S)- 2- phenylcyclopropyl]urea | 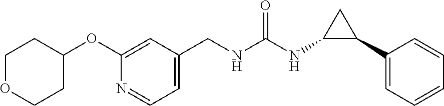 [SMILES: O=C(NCc1ccnc(OC2CCOCC2)c1)N[C@@H]1C[C@H]1c1ccccc1] | 5.93 | 58 |
| 21 | 1-[[2- (difluoromethyl)pyridin- 4-yl]methyl]-3- [(1R,2S)-2- phenylcyclopropyl]urea | 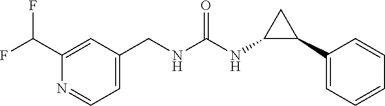 [SMILES: O=C(NCc1ccnc(C(F)F)c1)N[C@@H]1C[C@H]1c1ccccc1] | 3.20 | 72 |
| 22 | 1-[(1R,2S)-2- phenylcyclopropyl]-3- [[2- (trifluoromethyl)pyridin- 4-yl]methyl]urea | 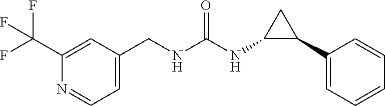 [SMILES: O=C(NCc1ccnc(C(F)(F)F)c1)N[C@@H]1C[C@H]1c1ccccc1] | 0.72 | 53 |
| 23 | 1-[[2-(1,3-oxazol-5- yl)pyridin-4-yl]methyl]- 3-[rac-(1R,2S)-2- cyclohexylcyclopropyl] urea | 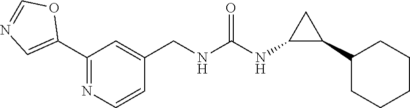 [SMILES: O=C(NCc1ccnc(-c2cnco2)c1)N[C@@H]1C[C@H]1C1CCCCC1] | 0.92 | 63 |
| 24 | 1-[rac-(1R,2S)-2- cyclohexylcyclopropyl]- 3-[[2-[4- (trifluoromethyl)imidazol- 1-yl]pyridin-4- yl]methyl]urea | 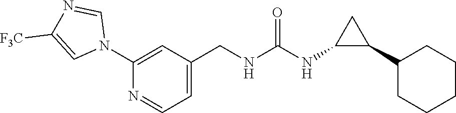 [SMILES: O=C(NCc1ccnc(-n2cnc(C(F)(F)F)c2)c1)N[C@@H]1C[C@H]1C1CCCCC1] | 0.16 | 45 |
| 25 | 1-[(2- cyclobutyloxypyridin- 4-yl)methyl]-3- [(1R,2S)-2- phenylcyclopropyl]urea | 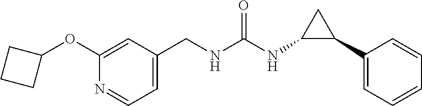 [SMILES: O=C(NCc1ccnc(OC2CCC2)c1)N[C@@H]1C[C@H]1c1ccccc1] | 1.05 | 60 |
| 26 | 1-[[2-(1,1- difluoroethyl)pyridin-4- yl]methyl]-3-[(1R,2S)- 2- phenylcyclopropyl]urea | 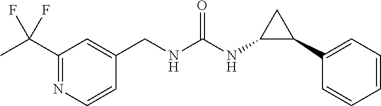 [SMILES: CC(F)(F)c1cc(CNC(=O)N[C@@H]2C[C@H]2c2ccccc2)ccn1] | 1.56 | 97 |
| 27 | 1-[(2-imidazol-1- ylpyridin-4-yl)methyl]- 3-[(1R,2S)-2- phenylcyclopropyl]urea | 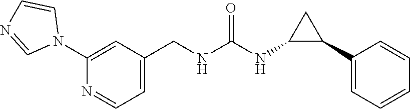 [SMILES: O=C(NCc1ccnc(-n2ccnc2)c1)N[C@@H]1C[C@H]1c1ccccc1] | 2.84 | 45 |
| 28 | 1-[(1R,2S)-2- phenylcyclopropyl]-3- [[2- (trifluoromethoxy)pyridin- 4-yl]methyl]urea | 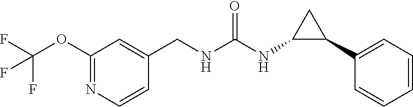 [SMILES: O=C(NCc1ccnc(OC(F)(F)F)c1)N[C@@H]1C[C@H]1c1ccccc1] | 1.36 | 62 |
| 29 | 1-[(1S)-1-(2- methoxypyridin-4- yl)ethyl]-3-[(1R,2S)-2- phenylcyclopropyl]urea | 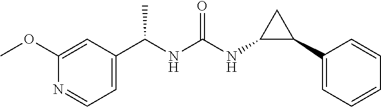 [SMILES: COc1cc([C@H](C)NC(=O)N[C@@H]2C[C@H]2c2ccccc2)ccn1] | 0.52 | 47 |
| 30 | 1-((S)-1-(2- (difluoromethoxy)pyridin- 4-yl)ethyl)-3- ((1R,2S)-2- phenylcyclopropyl)urea | 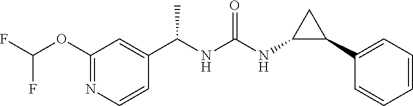 [SMILES: C[C@H](NC(=O)N[C@@H]1C[C@H]1c1ccccc1)c1ccnc(OC(F)F)c1] | 0.21 | 72 |
| 31 | 1-[(1R,2S)-2- phenylcyclopropyl]-3- [[2-[4- (trifluoromethyl)imidazol- 1-yl]pyridin-4- yl]methyl]urea | 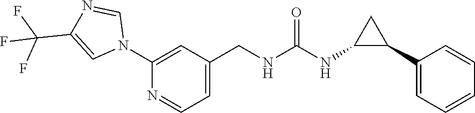 [SMILES: O=C(NCc1ccnc(-n2cnc(C(F)(F)F)c2)c1)N[C@@H]1C[C@H]1c1ccccc1] | 0.50 | 44 |
| 32 | 1-[2-(oxetan-3- yl)pyridin-4-yl]methyl]- 3-[(1R,2S)-2- phenylcyclopropyl]urea | 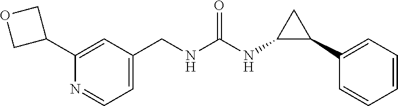 [SMILES: O=C(NCc1ccnc(C2COC2)c1)N[C@@H]1C[C@H]1c1ccccc1] | 3.02 | 40 |
| 33 | 1-[(1R,2S)-2- phenylcyclopropyl]-3- [(1S)-1-[2-[4- (trifluoromethyl)imidazol- 1-yl]pyridin-4- yl]ethyl]urea | 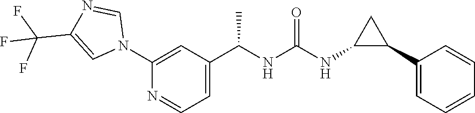 [SMILES: C[C@H](NC(=O)N[C@@H]1C[C@H]1c1ccccc1)c1ccnc(-n2cnc(C(F)(F)F)c2)c1] | 0.31 | 40 |
| 34 | 1-[(2-phenoxypyridin- 4-yl)methyl]-3- [(1R,2S)-2- phenylcyclopropyl]urea | 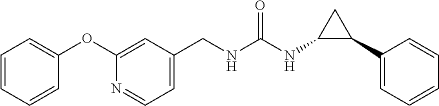 [SMILES: O=C(NCc1ccnc(Oc2ccccc2)c1)N[C@@H]1C[C@H]1c1ccccc1] | 4.78 | 62 |
| 35 | 1-[[2-(2,2- difluoroethoxy)pyridin- 4-yl]methyl]-3- [(1R,2S)-2- phenylcyclopropyl]urea | 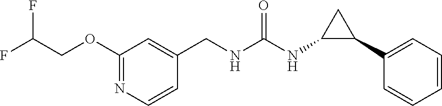 [SMILES: O=C(NCc1ccnc(OCC(F)F)c1)N[C@@H]1C[C@H]1c1ccccc1] | 0.20 | 66 |
| 36 | 1-[[2- (difluoromethoxy)pyridin- 4-yl]methyl]-3-[rac- (1R,2S)-1-methyl-2- phenylcyclopropyl]urea | 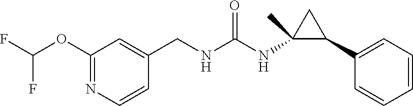 [SMILES: C[C@@]1(NC(=O)NCc2ccnc(OC(F)F)c2)C[C@H]1c1ccccc1] | 0.12 | 69 |
| 37 | 1-[2- (fluoromethoxy)pyridin- 4-yl]methyl]-3- [(1R,2S)-2- phenylcyclopropyl]urea | 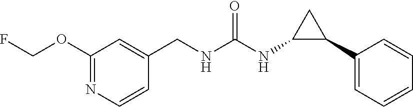 [SMILES: O=C(NCc1ccnc(OCF)c1)N[C@@H]1C[C@H]1c1ccccc1] | 0.91 | 74 |
| 38 | 1-[1-[2- (difluoromethoxy)pyridin- 4-ylethyl]-3- [(1R,2S)-2- phenylcyclopropyl]urea | 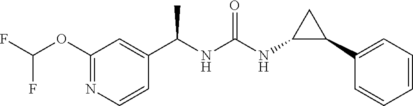 [SMILES: C[C@@H](NC(=O)N[C@@H]1C[C@H]1c1ccccc1)c1ccnc(OC(F)F)c1] | 3.81 | 42 |
| 39 | 1-[(1R,2S)-2- phenylcyclopropyl]-3- [[2-[3- (trifluoromethyl)pyrazol- 1-yl]pyridin-4- yl]methyl]urea | 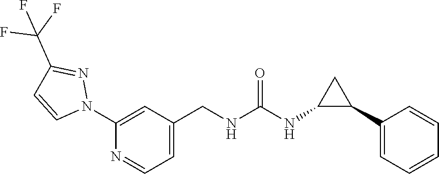 [SMILES: O=C(NCc1ccnc(-n2ccc(C(F)(F)F)n2)c1)N[C@@H]1C[C@H]1c1ccccc1] | 0.07 | 78 |
| 40 | 1-[rac-(1R,2S)-2- cyclohexylcyclopropyl]- 3-[[2-(1,2-thiazol-4- yl)pyridin-4- yl]methyl]urea | 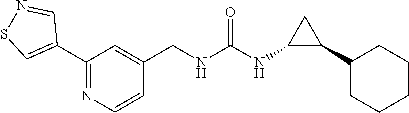 [SMILES: O=C(NCc1ccnc(-c2cnsc2)c1)N[C@@H]1C[C@H]1C1CCCCC1] | 0.78 | 57 |
| 41 | 1-[[2- (difluoromethoxy)pyridin- 4-yl]methyl]-3- [(1R,2R)-2- (trifluoromethyl) cyclopropyl]urea | 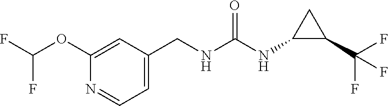 [SMILES: O=C(NCc1ccnc(OC(F)F)c1)N[C@@H]1C[C@H]1C(F)(F)F] | 0.23 | 54 |
| 42 | 1-[[2- (difluoromethoxy)pyridin- 4-yl]methyl]-3-[rac- (1R,3R)-2,2-difluoro-3- methylcyclopropyl]urea | 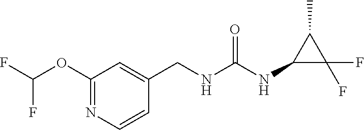 [SMILES: C[C@H]1[C@H](NC(=O)NCc2ccnc(OC(F)F)c2)C1(F)F] | 1.03 | 58 |
| 43 | 1-[[2- (difluoromethoxy)pyridin- 4-yl]methyl]-3-[rac- (1R,2R)-2-(oxolan-2- yl)cyclopropyl]urea | 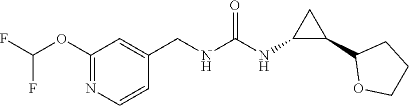 [SMILES: O=C(NCc1ccnc(OC(F)F)c1)N[C@@H]1C[C@H]1C1CCCO1] | 8.01 | 61 |
| 44 | 1-[[2- (difluoromethoxy)pyridin- 4-yl]methyl]-3-[rac- (1R,2R)-2-(1,1- difluoroethyl) cyclopropyl]urea | 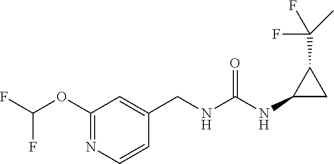 [SMILES: CC(F)(F)[C@@H]1C[C@H]1NC(=O)NCc1ccnc(OC(F)F)c1] | 0.33 | 40 |
| 45 | 1-(2- cyclohexylcyclopropyl)- 3-[[2-(oxan-4- yloxy)pyridin-4- yl]methyl]urea | 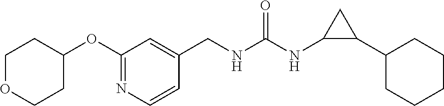 [SMILES: O=C(NCc1ccnc(OC2CCOCC2)c1)NC1CC1C1CCCCC1] | 0.88 | 77 |
| 46 | 1-[[2- (difluoromethoxy)pyridin- 4-yl]methyl]-3-[rac- (1R,2R)-2- methylcyclopropyl]urea | 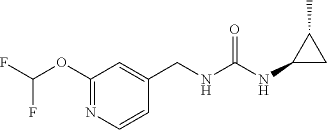 [SMILES: C[C@@H]1C[C@H]1NC(=O)NCc1ccnc(OC(F)F)c1] | 0.90 | 51 |
| 47 | 1-[[2- (difluoromethoxy)pyridin- 4-yl]methyl]-3-[rac- (1R,2R)-2-methyl-2- (trifluoromethyl) cyclopropyl]urea | 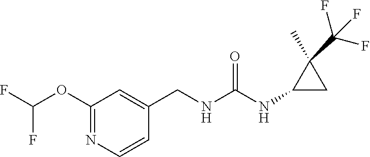 [SMILES: C[C@]1(C(F)(F)F)C[C@@H]1NC(=O)NCc1ccnc(OC(F)F)c1] | 0.05 | 77 |
| 48 | 1-[[2-(2,3- difluoropropoxy)pyridin- 4-yl]methyl]-3- [(1R,2S)-2- phenylcyclopropyl]urea | 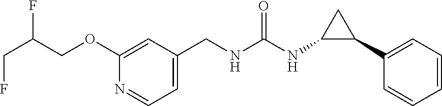 [SMILES: O=C(NCc1ccnc(OCC(F)CF)c1)N[C@@H]1C[C@H]1c1ccccc1] | 0.57 | 70 |
| 49 | 1-[[2- (difluoromethoxy) pyrimidin-4-yl]methyl]-3- [(1R,2R)-2- (trifluoromethyl) cyclopropyl]urea | 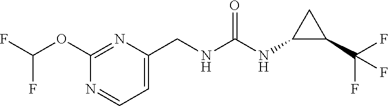 [SMILES: O=C(NCc1ccnc(OC(F)F)n1)N[C@@H]1C[C@H]1C(F)(F)F] | 3.83 | 42 |
| 50 | 1-[dideuterio-[2- (difluoromethoxy)pyridin- 4-yl]methyl]-3-[rac- (1R,2R)-2- (trifluoromethyl) cyclopropyl]urea | 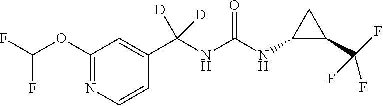 [SMILES: [2H]C([2H])(NC(=O)N[C@@H]1C[C@H]1C(F)(F)F)c1ccnc(OC(F)F)c1] | 0.62 | 47 |
| 51 | 1-[rac-(1R,2R)-2- (trifluoromethyl) cyclopropyl]-3-[[6-(2,2,2- trifluoroethoxy)pyrimidin- 4-yl]methyl]urea | 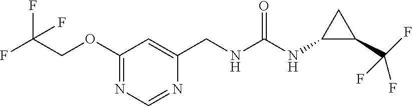 [SMILES: O=C(NCc1cc(OCC(F)(F)F)ncn1)N[C@@H]1C[C@H]1C(F)(F)F] | 0.28 | 52 |
| 52 | 1-[[6- (difluoromethoxy) pyrimidin-4-yl]methyl]-3- [(1S,2S)-2- (trifluoromethyl) cyclopropyl]urea | 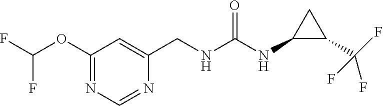 [SMILES: O=C(NCc1cc(OC(F)F)ncn1)N[C@H]1C[C@@H]1C(F)(F)F] | 1.00 | 43 |
| 53 | 1-[[2-[(2R)-1,1,1- trifluoro-3- methoxypropan-2- yl]oxypyridin-4- yl]methyl]-3-[rac- (1R,2R)-2- (trifluoromethyl) cyclopropyl]urea | 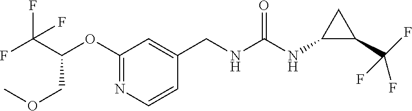 [SMILES: COC[C@@H](Oc1cc(CNC(=O)N[C@@H]2C[C@H]2C(F)(F)F)ccn1)C(F)(F)F] | 0.36 | 69 |

The compounds of the invention have shown to be agents acting on Kv7.2 and are therefore useful for the treatment and/or prophylaxis of any of the diseases, disorders, or disabilities described herein. They are in particular useful for the therapeutic and/or prophylactic treatment of a disorder, disease, or disabilities associated with Kv7.2. More particularly, they are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus. The behavioral disorder is for example Attention Deficit Hyperactivity Disorder (ADHD). The mood disorder is for example depression. The neurodevelopment disorder is for example autism spectrum disorder (ASD) or a syndromic developmental disorder. The syndromic developmental disorder is for example Dup15q syndrome (Dup15q), Fragile X syndrome (FXS), and Angelman syndrome. The epilepsies are for example broad pediatric epilepsy, West syndrome, Ohtahara syndrome and epileptic encephalopathy. Neurodegenerative diseases are for example Alzheimer's disease, or motor neuron diseases.

The compounds of the invention are therefore useful Kv7.2 modulators that provide for favorable pharmacological properties, such as potency, selectivity, and metabolic stability.

Compounds having one or more of the following combinations of features are found to display particulary beneficial Kv7.2 EC₅₀ values and/or Kv7.5_7.3/Kv7.2 selectivity ratios in the range of: (i) EC₅₀<3 μM, Select.>10×, or (ii) EC₅₀<1 μM, Select.>30×.

Compounds of this invention, in particular those with human liver microsomal clearance rate <20 μl/min/mg, are found to display beneficial metabolic stability.

In one more preferred embodiment of the invention, the compounds of this invention have the formula

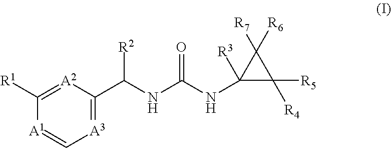

wherein
A¹ and A³ are N, and A² is CH; or
A¹ is N, A² and A³ are CH;
R¹ is haloC₁₋₆alkoxy;
R² is hydrogen;
R³ is hydrogen;
R⁴ and R⁵ are hydrogen;
R⁶ haloC₁₋₆alkyl; and
R⁷ is hydrogen.

In another more preferred embodiment of the invention, the compounds of this invention have the formula

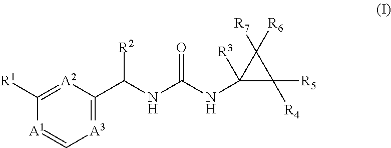

wherein
A¹ and A³ are N, and A² is CH; or
A¹ is N, A² and A³ are CH;
R¹ is haloC₁₋₆alkoxy selected from CHF₂O—, CFH₂O—, CF₃O—, CHF₂CH₂O—, FCH₂CFHCH₂O—, and CF₃CH₂O—;
R² is hydrogen;
R³ is hydrogen;
R⁴ and R⁵ are hydrogen;
R⁶ haloC₁₋₆alkyl selected from CF₃—, CHF₂—, CH₃CF₂—, and CFH₂—; and
R⁷ is hydrogen.

In another more preferred embodiment of the invention, the compounds of this invention have the formula

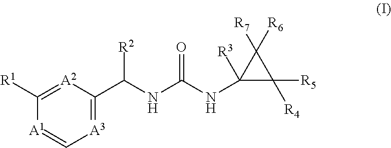

wherein
A¹ and A³ are N, and A² is CH; or
A¹ is N, A² and A³ are CH;
R¹ is haloC₁₋₆alkoxy selected from CHF₂O— and CF₃CH₂O—;
R² is hydrogen;
R³ is hydrogen;
R⁴ and R⁵ are hydrogen;
R⁶ haloC₁₋₆alkyl selected from CF₃ and CH₃CF₂—; and
R⁷ is hydrogen.

In one more preferred embodiment of the invention, the compounds of this invention have the formula (LX):

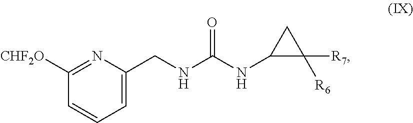

wherein R⁶ is selected from phenyl, saturated monocyclic 3-6 membered cycloalkyl, C₁₋₆alkyl, 4-6 membered heterocycloalkyl, and haloC₁₋₆alkyl, all as described herein, wherein phenyl, cycloalkyl, or heterocycloalkyl are optionally substituted with one, two, or three substituents independently selected from halogen, haloC₁₋₆alkyl, and haloC₁₋₆alkoxy;
and R⁷ is selected from H, C₁₋₆alkyl, haloC₁₋₆alkyl, and halogen.

In one particularly preferred embodiment of the invention, the compounds of this invention have the formula (X):

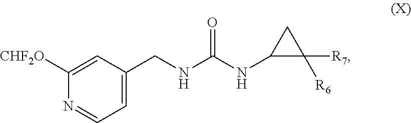

wherein R⁶ is selected from phenyl, saturated monocyclic 3-6 membered cycloalkyl and haloC₁₋₆alkyl, all as described herein; and R⁷ is selected from H, C₁₋₆alkyl, haloC₁₋₆alkyl, and halogen.

In some of the preferred embodiments, wherein the compounds have the formula (X), R⁶ is unsubstituted phenyl, and R⁷ is H or C₁₋₆alkyl.

In some of the preferred embodiments, wherein the compounds have the formula (X), R⁶ is a unsubstituted saturated monocyclic 3-6 membered cycloalkyl, and R⁷ is H or C₁₋₆alkyl.

In some of the preferred embodiments, wherein the compounds have the formula (X), R⁶ is a unsubstituted saturated monocyclic 3-6 membered cycloalkyl selected from cyclopropyl, cyclobutyl, cyclopentyl, and cyclohexyl, and R⁷ is H or C₁₋₆alkyl.

In some of the preferred embodiments, wherein the compounds have the formula (X), R⁶ is haloC₁₋₆alkyl selected from (CH₃)₂FC—, CF₃CH₂—, CH₃CF₂—, CF₃—, CH₂F—, and CHF₂—, and R⁷ is H or C₁₋₆alkyl.

In some of the preferred embodiments, wherein the compounds have the formula (X), R⁶ is CH₃CF₂—, and R⁷ is H or C₁₋₆alkyl.

In one particularly preferred embodiment, the invention provides a compound of formula (X), or a solvate, or a pharmaceutically acceptable salt thereof, wherein R⁶ is CH₃CF₂— with a structure selected from:

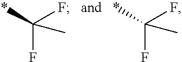

and R⁷ is H or C₁₋₆alkyl.

In one particularly preferred embodiment, the invention provides a compound of formula (X), or a solvate, or a pharmaceutically acceptable salt thereof, wherein R⁶ is CH₃CF₂— with a structure:

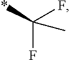

and R⁷ is H or C₁₋₆alkyl.

In one particularly preferred embodiment, the invention provides a compound of formula (X), or a solvate, or a pharmaceutically acceptable salt thereof, wherein R⁶ is CF₃—.

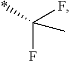

In one particularly preferred embodiment, the invention provides a compound of formula (X), or a solvate, or a pharmaceutically acceptable salt thereof, wherein R⁶ is CF₃— having the structures:

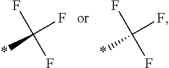

and R⁷ is H or C₁₋₆alkyl.

In one particularly preferred embodiment, the invention provides a compound of formula (X), or a solvate, or a pharmaceutically acceptable salt thereof, wherein R⁶ is CF₃— having the structure:

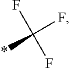

and R⁷ is H or C₁₋₆alkyl.

In one particularly preferred embodiment, the invention provides a compound of formula (X), or a solvate, or a pharmaceutically acceptable salt thereof, wherein R⁶ is CF₃— having the structure:

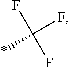

and R⁷ is H or C₁₋₆alkyl.

In one preferred embodiment of the invention, the compounds of this invention have the formula (XI):

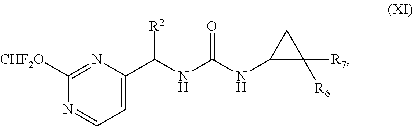

wherein R⁶ is selected from phenyl, saturated monocyclic 3-6 membered cycloalkyl, C₁₋₆alkyl, 4-6 membered heterocycloalkyl, and haloC₁₋₆alkyl, all as described herein, wherein phenyl, cycloalkyl, or heterocycloalkyl are optionally substituted with one, two, or three substituents independently selected from halogen, haloC₁₋₆alkyl, and haloC₁₋₆alkoxy; and R⁷ is selected from H, C₁₋₆alkyl, haloC₁₋₆alkyl, and halogen.

In some of the preferred embodiments, wherein the compounds have the formula (XI), wherein R⁶ is unsubstituted phenyl, and R⁷ is H or C₁₋₆alkyl.

In some of the preferred embodiments, wherein the compounds have the formula (XI), wherein R⁶ is a unsubstituted saturated monocyclic 3-6 membered cycloalkyl, and R⁷ is H or C₁₋₆alkyl.

In some of the preferred embodiments, wherein the compounds have the formula (XI), wherein R⁶ is a unsubstituted saturated monocyclic 3-6 membered cycloalkyl selected from cyclopropyl, cyclobutyl, cyclopentyl, and cyclohexyl, and R⁷ is H or C₁₋₆alkyl.

In some of the preferred embodiments, wherein the compounds have the formula (XI), R⁶ is haloC₁₋₆alkoxy selected from (CH₃)₂FC—, CF₃CH₂—, CH₃CF₂—, CF₃—, CH₂F—, and CHF₂—, and R₇ is H or C₁₋₆alkyl.

In one particularly preferred embodiment, the invention provides a compound of formula (XI), or a solvate, or a pharmaceutically acceptable salt thereof, wherein R⁶ is CF₃CH₂—, and R⁷ is H or C₁₋₆alkyl.

In one particularly preferred embodiment, the invention provides a compound of formula (XI), or a solvate, or a pharmaceutically acceptable salt thereof, wherein R⁶ is CF₃CH₂— with a structure selected from:

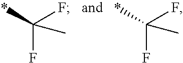

and R⁷ is H or C₁₋₆alkyl.

In one particularly preferred embodiment, the invention provides a compound of formula (XI), or a solvate, or a pharmaceutically acceptable salt thereof, wherein R⁶ is CF₃CH₂— with a structure:

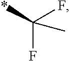

and R⁷ is H or C₁₋₆alkyl.

In one particularly preferred embodiment, the invention provides a compound of formula (XI), or a solvate, or a pharmaceutically acceptable salt thereof, wherein R⁶ is CF₃CH₂— with a structure:

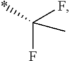

In one particularly preferred embodiment, the invention provides a compound of formula (XI), or a solvate, or a pharmaceutically acceptable salt thereof, wherein R⁶ is CF₃—, and R⁷ is H or C₁₋₆alkyl.

In one particularly preferred embodiment, the invention provides a compound of formula (XI), or a solvate, or a pharmaceutically acceptable salt thereof, wherein R⁶ is CF₃— having the structures:

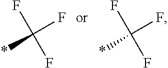

and R⁷ is H or C₁₋₆alkyl.

In one particularly preferred embodiment, the invention provides a compound of formula (XI), or a solvate, or a pharmaceutically acceptable salt thereof, wherein R⁶ is CF₃— having the structure:

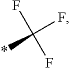

and R₇ is H or C₁₋₆alkyl.

In one particularly preferred embodiment, the invention provides a compound of formula (XI), or a solvate, or a pharmaceutically acceptable salt thereof, wherein R⁶ is CF₃— having the structure:

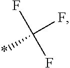

and R⁷ is H or C₁₋₆alkyl.

In some preferred embodiments, the compound of formula (I), or a solvate, or a pharmaceutically acceptable salt thereof is selected from Table 2 (Kv7.2 EC₅₀<3 μM and; selectivity Kv7.5_7.3/Kv7.2)>10×.

TABLE 2

| | | |
|---|---|---|
| 3 | 1-[(6-chloropyridin-2- yl)methyl]-3-[rac-(1R,2S)-2- cyclohexylcyclopropyl]urea | 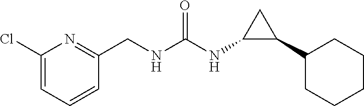 [SMILES: O=C(NCc1cccc(Cl)n1)N[C@@H]1C[C@H]1C1CCCCC1] |
| 10 | 1-[(2-cyanopyridin-4- yl)methyl]-3-[(1R,2S)-2- phenylcyclopropyl]urea | 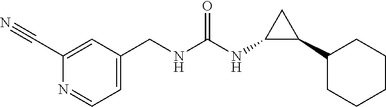 [SMILES: N#Cc1cc(CNC(=O)N[C@@H]2C[C@H]2C2CCCCC2)ccn1] |
| 11 | 1-(2- cyclohexylcyclopropyl)-3- [(2-imidazol-1-ylpyridin-4- yl)methyl]urea | 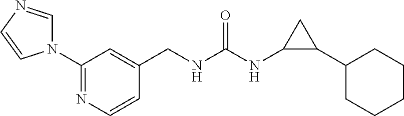 [SMILES: O=C(NCc1ccnc(-n2ccnc2)c1)NC1CC1C1CCCCC1] |
| 12 | 1-[(2-pyrazol-1- ylpyridin-4-yl)methyl]-3- [rac-(1R,2S)-2- phenylcyclopropyl]urea | 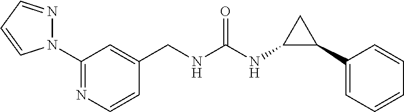 [SMILES: O=C(NCc1ccnc(-n2cccn2)c1)N[C@@H]1C[C@H]1c1ccccc1] |
| 15 | 1-[(1S,2R)-2- cyclohexylcyclopropyl]-3- [(2-imidazol-1-ylpyridin-4- yl)methyl]urea | 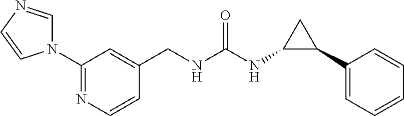 [SMILES: O=C(NCc1ccnc(-n2ccnc2)c1)N[C@@H]1C[C@H]1c1ccccc1] |
| 17 | 1-[(2-chloropyridin-4- yl)methyl]-3-[rac-(1R,2S)-2- cyclohexylcyclopropyl]urea | 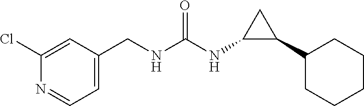 [SMILES: O=C(NCc1ccnc(Cl)c1)N[C@@H]1C[C@H]1C1CCCCC1] |
| 23 | 1-[2-(1,3-oxazol-5- yl)pyridin-4-yl]methyl]-3- [rac-(1R,2S)-2- cyclohexylcyclopropyl]urea | 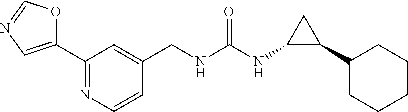 [SMILES: O=C(NCc1ccnc(-c2cnco2)c1)N[C@@H]1C[C@H]1C1CCCCC1] |
| 25 | 1-[(2- cyclobutyloxypyridin-4- yl)methyl]-3-[(1R,2S)-2- phenylcyclopropyl]urea | 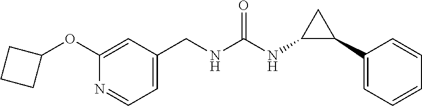 [SMILES: O=C(NCc1ccnc(OC2CCC2)c1)N[C@@H]1C[C@H]1c1ccccc1] |
| 27 | 1-[(2-imidazol-1- ylpyridin-4-yl)methyl]-3- [(1R,2S)-2- phenylcyclopropyl]urea | 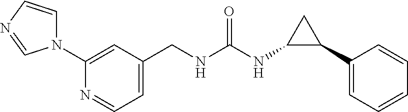 [SMILES: O=C(NCc1ccnc(-n2ccnc2)c1)N[C@@H]1C[C@H]1c1ccccc1] |
| 37 | 1-[[2- (fluoromethoxy)pyridin-4- yl]methyl]-3-[(1R,2S)-2- phenylcyclopropyl]urea | 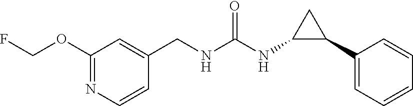 [SMILES: O=C(NCc1ccnc(OCF)c1)N[C@@H]1C[C@H]1c1ccccc1] |
| 39 | 1-[(1R,2S)-2- phenylcyclopropyl]-3-[2-[3- (trifluoromethyl)pyrazol-1- yl]pyridin-4-yl]methyl]urea | 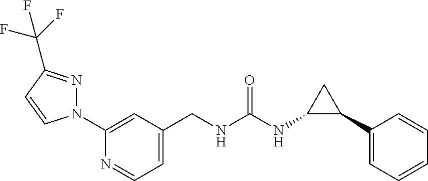 [SMILES: O=C(NCc1ccnc(-n2ccc(C(F)(F)F)n2)c1)N[C@@H]1C[C@H]1c1ccccc1] |
| 40 | 1-[rac-(1R,2S)-2- cyclohexylcyclopropyl]-3- [[2-(1,2-thiazol-4-yl)pyridin- 4-yl]methyl]urea | 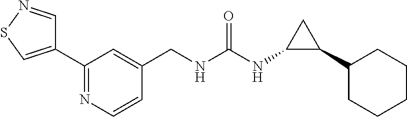 [SMILES: O=C(NCc1ccnc(-c2cnsc2)c1)N[C@@H]1C[C@H]1C1CCCCC1] |
| 41 | 1-[[2- (difluoromethoxy)pyridin-4- yl]methyl]-3-[(1R,2R)-2- (trifluoromethyl)cyclopropyl] urea | 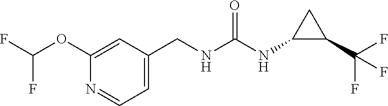 [SMILES: O=C(NCc1ccnc(OC(F)F)c1)N[C@@H]1C[C@H]1C(F)(F)F] |
| 44 | 1-[2- (difluoromethoxy)pyridin-4- yl]methyl]-3-[rac-(1R,2R)-2- (1,1- difluoroethyl)cyclopropyl]urea | 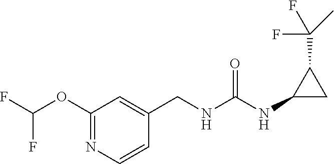 [SMILES: CC(F)(F)[C@@H]1C[C@H]1NC(=O)NCc1ccnc(OC(F)F)c1] |
| 45 | 1-(2- cyclohexylcyclopropyl)-3- [[2-(oxan-4-yloxy)pyridin-4- yl]methyl]urea | 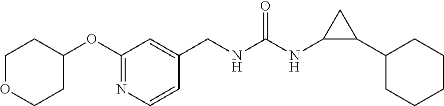 [SMILES: O=C(NCc1ccnc(OC2CCOCC2)c1)NC1CC1C1CCCCC1] |
| 46 | 1-[[2- (difluoromethoxy)pyridin-4- yl]methyl]-3-[rac-(1R,2R)-2- methylcyclopropyl]urea | 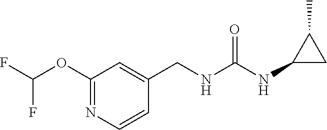 [SMILES: C[C@@H]1C[C@H]1NC(=O)NCc1ccnc(OC(F)F)c1] |
| 50 | 1-[dideuterio-[2- (difluoromethoxy)pyridin-4- yl]methyl]-3-[rac-(1R,2R)-2- (trifluoromethyl)cyclopropyl] urea | 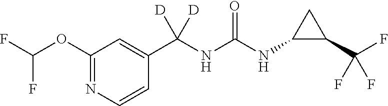 [SMILES: [2H]C([2H])(NC(=O)N[C@@H]1C[C@H]1C(F)(F)F)c1ccnc(OC(F)F)c1] |
| 51 | 1-[rac-(1R,2R)-2- (trifluoromethyl)cyclopropyl]- 3-[[6-(2,2,2- trifluoroethoxy)pyrimidin-4- yl]methyl]urea | 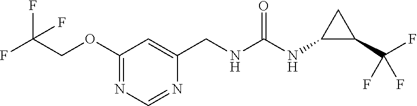 [SMILES: O=C(NCc1cc(OCC(F)(F)F)ncn1)N[C@@H]1C[C@H]1C(F)(F)F] |
| 52 | 1-[[6- (difluoromethoxy)pyrimidin- 4-yl]methyl]-3-[(1S,2S)-2- (trifluoromethyl)cyclopropyl] urea | 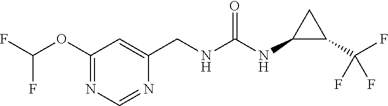 [SMILES: O=C(NCc1cc(OC(F)F)ncn1)N[C@H]1C[C@@H]1C(F)(F)F] |

Compounds of formula (I) or (I′) having one or more of the following combinations of features are found to display particulary beneficial Kv7.2 EC₅₀ values and/or Kv7.5_7.3/Kv7.2 selectivity ratios in the range of: EC₅₀<3 μM, Select.>10×, EC₅₀<1 μM, Select.>30× or EC₅₀<1 μM, Select.>30× with favorable metabolic stability.

Measurements have shown that compounds of formula (I) or formula (I′), or solvates or pharmaceutical salts thereof, have favorable pharmacological properties as described herein if the Kv7.2 EC₅₀<3 μM and selectivity ratio Kv7.5_7.3/Kv7.2>10×.

Measurements have also shown that compounds of formula (I) or formula (I′), or solvates or pharmaceutical salts thereof, have favorable pharmacological properties as described herein, and are therefore preferred, if they demonstrate one or more of the following properties: Kv7.5_7.3/Kv7.2 selectivity ratio >10×; and/or
human liver microsomal clearance rate <20 μl/min/mg; and/or
Kv7.4/Kv7.2 selectivity >10×.

In some embodiments of the invention, in the compounds of this invention one or more hydrogen atoms is (are) replaced by a deuterium. It has been surprisingly found that deuteration of the compounds of this invention offer the advantage of retaining the pharmacological profile of their hydrogen counterparts while positively impacting their metabolic outcome. Selective replacement of one or more hydrogen with deuterium, in the compounds of the present invention, improves the pharmaceutical profile of compounds of this invention, e.g. by reducing the amount of undesired metabolites when compared to its all hydrogen counterparts and by lowering rates of metabolim, and hence increasing half-life.

Methods for incorporation of deuterium into compounds are well established. Using metabolic studies established in the art, the compound of the present invention can be tested to identify sites for selective placement of a deuterium isotope, which isotope will not be metabolized or be metabolized at a lower rate. Moreover, these studies identify sites of metabolism as the location where a deuterium atom would be placed.

In one embodiment, the present invention provides a compound of this invention, wherein one or more of the hydrogen atoms are replaced with deuterium.

In one embodiment, the present invention provides a compound of this invention, the compound of formula (I*), or a solvate, or a pharmaceutically acceptable salt thereof:

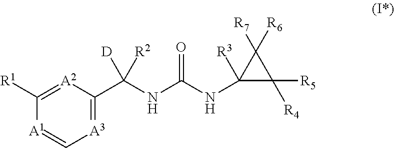

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is selected from H, D, or C₁₋₆alkyl;
R³ is H, or C₁₋₆alkyl;
R⁴ is H, D, or halogen;
R⁵ is H, D, or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one preferred embodiment, the present invention provides a compound of this invention, the compound of formula (II*), or a solvate, or a pharmaceutically acceptable salt thereof:

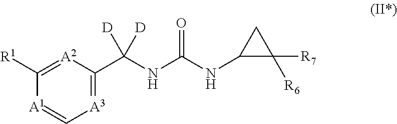

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In some preferred embodiments of the compound of formula (I*), R² is deuterium.

In some preferred embodiments of the compound of formula (I*) or (II*), R¹ is haloC₁₋₆ alkoxy as described herein, R² is deuterium, R³ is H, R⁴ and R⁵ are H, and R⁶ is selected from phenyl, saturated monocyclic 3-6 membered cycloalkyl, and haloC₁₋₆alkyl, all as described herein, and R⁷ is H or C₁₋₆alkyl.

In some preferred embodiments of the compound of formula (II*), R¹ is haloC₁₋₆alkoxy as described herein, and R⁶ is selected from phenyl, saturated monocyclic 3-6 membered cycloalkyl, and haloC₁₋₆alkyl, all as described herein, and R⁷ is H or C₁₋₆alkyl.

In some more preferred embodiments of the compound of formula (II*), R¹ is haloC₁₋₆alkoxy as described herein, and R⁶ is, and haloC₁₋₆alkyl, all as described herein, and R⁷ is H.

In one embodiment, the invention provides a compound of formula (I′), or a solvate, or a pharmaceutically acceptable salt thereof, wherein R² is selected from H, D, and C₁₋₆alkyl and wherein R¹ is selected from H, D, and C₁₋₆alkyl.

In one embodiment, the invention provides a compound of formula (I′), or a solvate, or a pharmaceutically acceptable salt thereof, wherein R² is H or D; and R⁸ is H, or D.

In one embodiment, the invention provides a compound of formula (I′), or a solvate, or a pharmaceutically acceptable salt thereof, wherein one of R² or R⁸ is C₁₋₆alkyl selected from CH₃—, CH₃CH₂—, CH₃CH₂CH₂—, (CH₃)₂CH₂—, (CH₃CH₂CH₂CH₂—), (CH₃)₂CH₂CH₂—, CH₃CH(CH₃)CH₂—, and (CH₃)₃C—.

In one embodiment, the invention provides a compound of formula (I′), or a solvate, or a pharmaceutically acceptable salt thereof, wherein one of R² or R⁸ is D.

### Additional Options for Variables

In all aspects, embodiments, claims and chemical formulae described herein, various lists of options are provided for the variables R¹ and R². In additional, distinct, aspects, embodiments and chemical formulae, these lists of options for R¹ and R² may each independently be expanded to include additional options in one or more of the lists associated with a given aspect, embodiment, or formula as provided below. These additional options may be included independently of the additional options for other variables in a formula, e.g. the additional options for one variable, such as R¹, may be included in a given Formula independently, without requiring inclusion of the additional options for any of the other variables in the molecule; and similarly for any and all of the other variables.

Throughout this specification, this has the effect of providing additional aspects, embodiments, claims and formulae in which the definitions of one or more of the variables R¹ and R² are expanded to encompass the following additional options.

In particular, these additional options are applicable to each of formulae (I′), (I), (II′), (I*), (II**), (II), (III), (IV), (V), (VI), (VII), (VIII), (IX), (X), (XI) etc.

Similarly, all options, groups, sub-groups, and combinations provided for the variables R¹, R², R³, R⁴, R⁵, R⁶ and R⁷ herein in respect of any of the formulae (I), (I*), (II**), (II), (III), (IV), (V), (VI), (VII), (VIII), (IX), (X), (XI) may also be applicable to any of formulae (I′) and (II′) alongside the options for R⁸ already stated in respect of formula (I′).

Throughout this specification, this has the effect of providing additional aspects, embodiments, claims and formulae in which the definitions of one or more of the variables R¹, R², R³, R⁴, R⁵, R⁶ and R⁷ are applied to any of formulae (I′) or (II′) alongside the options for R⁸ as already stated in respect of formula (I′).

### Additional Options for R¹

In addition to the options defined herein for R¹ in any of the various lists, including in the most general lists and in narrower lists, such as preferred, more preferred, and most preferred lists, the options for R¹ may also include:
haloC₁₋₆alkoxy optionally substituted with C₁₋₆alkoxy. The options and preferences for the haloC₁₋₆alkoxy mentioned elsewhere herein apply equally. The C₁₋₆alkoxy substituent is preferably C₁₋₃alkoxy, more preferably methoxy.

A specific additional option for R¹ is:

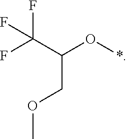

### Additional Options for R²

In addition to the options defined herein for R² in any of the various lists, including in the most general lists and in narrower lists, such as preferred, more preferred, and most preferred lists, the options for R² may also include.
D.

### Formula (I′)

All options, groups, sub-groups, and combinations provided in relation to formula (I) herein shall also apply to compounds of formula (I′) alongside the options stated for R¹. In particular preferences, R¹ is selected from H, D, and C₁₋₆alkyl. Preferably R¹ is D. Preferably R⁸ is D in combination with R² being D.

In some embodiments, if falling under the claims of this invention, the compounds of this invention do not include:

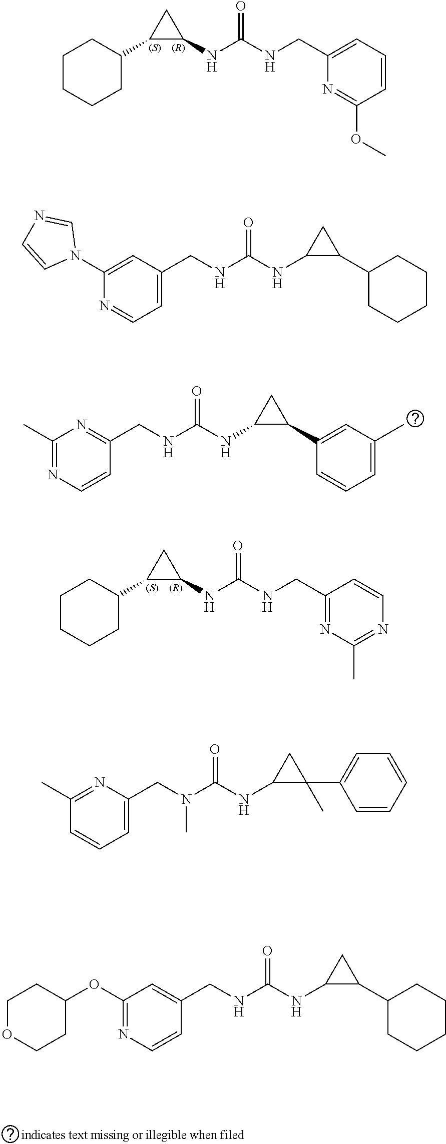

In one embodiment, the invention provides a compound of formula (I), or a solvate or a pharmaceutically acceptable salt thereof:

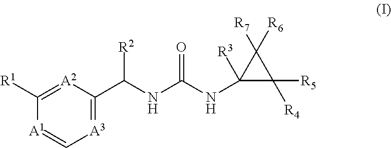

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyloxy, heterocycloalkyl, cycloalkyloxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
excluding

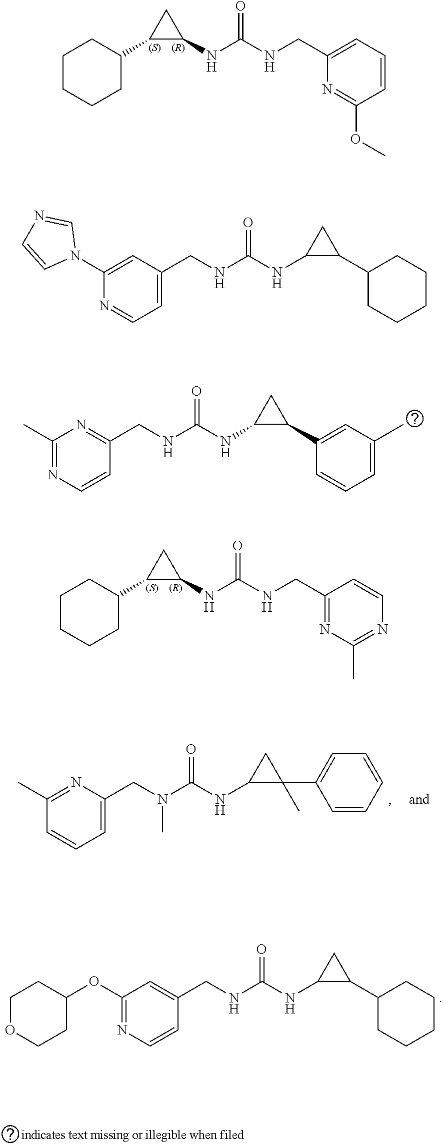

### Pharmaceutical Compositions and Administration

In one embodiment, the present invention provides a pharmaceutical composition comprising a compound of this invention as described herein.

In one embodiment, the present invention provides a pharmaceutical composition comprising a compound of this invention as described herein and one or more pharmaceutically acceptable excipients.

In one embodiment, the present invention provides a pharmaceutical composition additionally comprising one or more pharmaceutical excipients selected form diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, and wet granulation agent.

In a particular embodiment, the invention provides pharmaceutical compositions as described above which are in particular useful for the therapeutic and/or prophylactic treatment of a disorder, disease, or disabilities associated with Kv7.2.

More particularly, the pharmaceutical compositions as described herein are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus.

The compounds of this invention can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of this invention can be processed with pharmaceutically inert, inorganic or organic adjuvants for the production of tablets, coated tablets, dragées and hard gelatin capsules.

Lactose, corn starch, or derivatives thereof, talc, stearic acid or its salts etc. can be used, for example, as such adjuvants for tablets, dragées, or hard gelatin capsules.

Suitable adjuvants for soft gelatin capsules are, for example, vegetable oils, waxes, fats, semi-solid substances, or liquid polyols, etc.

Suitable adjuvants for the production of solutions and syrups are, for example, water, polyols, saccharose, invert sugar, or glucose, etc.

Suitable adjuvants for injection solutions are, for example, water, alcohols, polyols, glycerol, or vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Provided herein are pharmaceutical compositions comprising a compound of this invention. In some embodiments the pharmaceutical compositions comprise one or more pharmaceutically acceptable excipients. Conventional procedures for the selection and preparation of suitable pharmaceutical compositions are described in, for example, “Pharmaceuticals—The Science of Dosage Form Designs,” M. E. Aulton, Churchill Livingstone, 1988, which is hereby incorporated by reference in its entirety.

Further provided is a process for the preparation of a pharmaceutical composition, comprising combining one or more compounds of this invention.

Further provided is a process for the preparation of a pharmaceutical composition, comprising combining one or more compounds of this invention with one or more pharmaceutically acceptable excipients. Pharmaceutical compositions may be prepared, for example, according to conventional dissolution, mixing, granulating, or coating methods, or combinations thereof. Such pharmaceutically acceptable excipients may include, for example, sugars (e.g., lactose, glucose, sucrose); starches (e.g., corn starch, potato starch); cellulose and its derivatives (e.g., sodium carboxymethyl cellulose, ethyl cellulose, cellulose acetate); powdered tragacanth; malt; gelatin; talc; cocoa butter and suppository waxes; oils (e.g., peanut oil, cottonseed oil, safflower oil, sesame oil, olive oil, corn oil, soybean oil); glycols (e.g., propylene glycol); polyethylene glycols (PEG); esters (e.g., ethyl oleate, ethyl laurate); agar; buffering agents (e.g., magnesium hydroxide, aluminum hydroxide); alginic acid; pyrogen-free water; isotonic saline; Ringer's solution; ethyl alcohol; phosphate buffer solutions; non-toxic compatible lubricants (e.g., sodium lauryl sulfate, magnesium stearate); coloring agents; releasing agents; coating agents; sweetening; and flavoring and perfuming agents. Preservatives and antioxidants can also be present in the pharmaceutical composition, according to the judgment of the formulator.

Depending on the intended mode of administration, the disclosed pharmaceutical compositions can be in solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. These modes may include systemic or local administration such as oral, nasal, parenteral (as by intravenous (both bolus and infusion), intramuscular, or subcutaneous injection), transdermal, vaginal, buccal, rectal, or topical (as by powders, ointments, or drops) administration modes. These modes may also include intracisternally, intraperitoneally, as an oral or nasal spray, or as a liquid aerosol or dry powder pharmaceutical composition for inhalation. In some embodiments, the pharmaceutical composition provided herein comprises one or more disclosed compounds, tautomers thereof, and/or pharmaceutically acceptable salts thereof, and is for oral administration. In other embodiments, the pharmaceutical composition is for intravenous administration.

Solid dosage forms for oral administration may include capsules (e.g., soft and hard-filled gelatin capsules), tablets, pills, powders, and granules. Solid dosage forms may be prepared, in some embodiments, with one or more coatings and/or shells such as release controlling coatings, for example enteric coatings. Solid dosage forms may be formulated to release the one or more disclosed compounds (or solvate, tautomer, or pharmaceutically acceptable salt thereof) only, or mostly, or preferentially in a certain part of the gastrointestinal tract, optionally in a delayed manner. Solid dosage forms may also include, for example, micro-encapsulated forms.

Liquid dosage forms for oral administration may include, for example, pharmaceutically acceptable emulsions, microemulsions, solutions, suspensions, syrups, and elixirs. Such liquid compositions may include, for example, a pharmaceutically acceptable excipient such as water or other solvents, solubilizing agents, emulsifiers, oils, polyethylene glycols and fatty acid esters, adjuvants, sweetening agents, flavoring agents, or perfuming agents, or any combinations thereof.

Injectible pharmaceutical compositions include, for example, sterile injectable aqueous compositions (e.g., solutions, suspensions, or emulsions), or oleaginous suspensions.

Injectable pharmaceutical compositions may comprise, in some embodiments, one or more solvents and/or diluents, such as water, Ringer's solution, U.S.P. and isotonic sodium chloride solution, sterile fixed oils, fatty acid, or any combinations thereof. In some embodiments, an injectible pharmaceutical composition may be prepared as a lyophilized powder, for example a lyophilized powder that is to be mixed with a liquid diluent prior to injection.

In some embodiments, it may be desirable to prolong the effect of one or more compounds as disclosed herein, or pharmaceutically acceptable salt thereof, from administration through subcutaneous or intramuscular injection. Such delay may be accomplished, for example, through the use of a liquid suspension of crystalline or amorphous material with poor water solubility; or dissolving or suspending the compound, or solvate, tautomer, or pharmaceutically acceptable salt thereof, in an oil vehicle; or through an injectable depot form comprising microencapsule matrixes comprising one or more biodegradable polymers.

Pharmaceutical compositions for rectal or vaginal administration may include suppositories that can be prepared, for example using a suitable non-irritating excipient such as cocoa butter, polyethylene glycol, or a suppository wax; or using a fatty emulsion or suspension.

Dosage forms for topical or transdermal administration may include, for example, ointments, pastes, creams, lotions, gels, powders, solutions, sprays, inhalants, or patches. Ophthalmic pharmaceutical compositions and ear drops may also be prepared.

The pharmaceutical compositions provided herein may be packaged in unit-dose or multidose containers, for example sealed ampoules or vials, and may be stored in a freeze-dried (lyophilized) condition requiring only the addition of the sterile liquid excipient (e.g., diluent, carrier, for example water) for injection immediately prior to use. Extemporaneous injection solutions and suspensions may be prepared from sterile powders, granules, or tablets of the kind described herein. Unit dosage formulations include those containing a daily dose or unit daily sub-dose, or an appropriate fraction thereof, of the active ingredient.

The subject matter further provides veterinary compositions comprising at least one active ingredient as herein defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Medical Use

The compounds of this invention or pharmaceutical compositions comprising the same, as described herein, may be useful as pharmaceuticals for the therapeutic and/or prophylactic treatment of a disorder, disease or disability associated with Kv7.2 in a subject in need thereof.

In one embodiment, the present invention provides a method for the therapeutic and/or prophylactic treatment of a disorder, disease, or disability associated with Kv7.2 in a subject in need thereof, comprising administering an effective amount of compounds of this invention or pharmaceutical compositions thereof, as described herein.

In one embodiment, the present invention provides a method for the therapeutic and/or prophylactic treatment of a disorder, disease, or disability associated with Kv7.2 in a subject in need thereof, comprising administering an effective amount of the compounds of this invention, or pharmaceutical compositions comprising the same, as described herein, wherein the pharmaceutical composition further comprises one or more pharmaceutically acceptable excipients.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

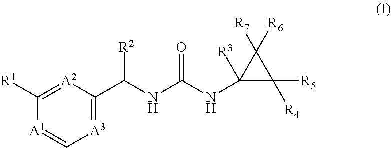

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the present invention provides a method for the therapeutic and/or prophylactic treatment of a disorder, disease, or disability associated with Kv7.2 in a subject in need thereof, comprising a compound of this invention, in particular of formulae (I)-(XI), or a solvate or a pharmaceutically acceptable salt thereof, any exemplified compound, any embodiment, or combinations of embodiments, as described herein.

In one embodiment, the present invention provides a method for the therapeutic and/or prophylactic treatment of a disorder, disease, or disability associated with Kv7.2 in a subject in need thereof, comprising a compound selected from any list of compounds as described herein, or solvates or pharmaceutically acceptable salts thereof.

In one embodiment, the present invention provides a method for the therapeutic and/or prophylactic treatment of a disorder, disease, or disability associated with Kv7.2 in a subject in need thereof, comprising a compound selected from Table 1 and 2, or a solvate or a pharmaceutically acceptable salt thereof.

In one embodiment, the present invention provides a method for the theraeutic and/or prophylactic treatment of a disorder, disease, or disability associated with Kv7.2 in a subject in need thereof, comprising a compound of formulae (I)-(XI), or a solvate or a pharmaceutically acceptable salt thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the above method for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the above method for the therapeutic and/or prophylactic treatment of a disorder, disease, or disability associated with Kv7.2 in a subject in need thereof, wherein the mood disorder is depression.

In one embodiment, the present invention provides the above method for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the above method for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the above method for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the above method for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a method for the therapeutic and/or prophylactic treatment of a disorder, disease, or disability associated with Kv7.2 in a subject in need thereof, comprising administering an effective amount of pharmaceutical compositions as described herein.

Further provided herein is a compound, a solvate, a pharmaceutically acceptable salt or a pharmaceutical composition thereof, as described herein, for use as therapeutically active substance.

In one embodiment, the present invention provides the use of a compound of formula (I), or a solvate or a pharmaceutically acceptable salt thereof:

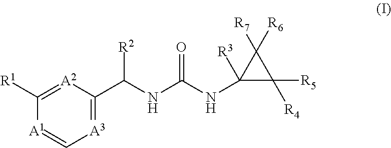

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
as therapeutically active substance.

In one embodiment, the present invention provides the use of a compound of this invention, in particular of formulae (I)-(XI), or a solvate or a pharmaceutically acceptable salt thereof, any exemplified compound, any embodiment, or combinations of embodiments, as described herein, as therapeutically active substance.

In one embodiment, the present invention provides the use of a compound selected from Table 1, and 2, or a solvate or a pharmaceutically acceptable salt thereof, as therapeutically active substance.

In one embodiment, the present invention provides the use of a compound selected from any list of compounds described herein, or a solvate or a pharmaceutically acceptable salt thereof, as therapeutically active substance.

Further provided herein is a compound, a solvate, a pharmaceutically acceptable salt, or a pharmaceutical composition thereof, for use in the therapeutic and/or prophylactic treatment of a disorder, disease, or disability associated with Kv7.2 in a subject in need thereof.

In one embodiment, the present invention provides a compound of formula (I), or a solvate or a pharmaceutically acceptable salt thereof:

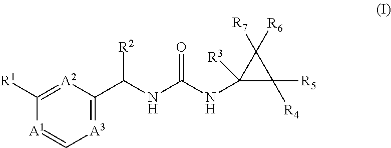

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
for use in the therapeutic and/or prophylactic treatment of a disorder, disease, or disability associated with Kv7.2 in a subject in need thereof.

In one embodiment, the present invention provides a compound of this invention, in particular of formulae (I)-(XI), or a solvate or a pharmaceutically acceptable salt thereof, any exemplified compound, any embodiment, or combinations of embodiments, as described herein, for use in the therapeutic and/or prophylactic treatment of a disorder, disease, or disability associated with Kv7.2 in a subject in need thereof.

In one embodiment, the present invention provides a compound selected from Table 1, and 2, or a solvate or a pharmaceutically acceptable salt thereof, for use in the therapeutic and/or prophylactic treatment of a disorder, disease, or disability associated with Kv7.2 in a subject in need thereof.

In one embodiment, the present invention provides a compound selected from any list of compounds of this invention, for use in the therapeutic and/or prophylactic treatment of a disorder, disease, or disability associated with Kv7.2 in a subject in need thereof.

In one embodiment, the present invention provides a compound of this invention for use in the therapeutic and/or prophylactic treatment of a disorder, disease, or disability associated with Kv7.2 in a subject in need thereof, wherein such disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a compound of this invention for use in the therapeutic and/or prophylactic treatment of a disorder, disease, or disability associated with Kv7.2 in a subject in need thereof, wherein the mood disorder is depression.

In one embodiment, the present invention provides a compound of this invention, for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a compound of this invention for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

Further provided herein is the use of a compound of this invention in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

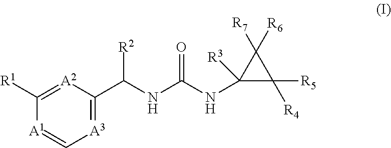

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
for the therapeutic and/or prophylactic treatment of a disorder, disease, or disability associated with Kv7.2 in a subject in need thereof.

In one embodiment, the present invention provides the use of a compound of this invention, in particular of formulae (I)-(XI), or solvate or a pharmaceutically acceptable salt thereof, any exemplified compound, any embodiment, or combinations of embodiments, as described herein, as described herein, for the therapeutic and/or prophylactic treatment of a disorder, disease, or disability associated with Kv7.2 in a subject in need thereof.

In one embodiment, the present invention provides the use of a compound selected from Table 1, and 2, or solvate or a pharmaceutically acceptable salt thereof, for the therapeutic and/or prophylactic treatment of a disorder, disease, or disability associated with Kv7.2 in a subject in need thereof.

In one embodiment, the invention provides the use a compound selected from any list of compounds described herein, or a solvate or a pharmaceutically acceptable salt thereof, for the therapeutic and/or prophylactic treatment of a disorder, disease, or disability associated with Kv7.2 in a subject in need thereof.

In one embodiment, the invention provides for the use of a compound of this invention in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the invention provides for the use of a compound of this invention in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the invention provides for the use of a compound of this invention in the therapeutic and/or prophylactic treatment of a disorder, disease, or disability associated with Kv7.2 in a subject in need thereof, wherein the mood disorder is depression.

In one embodiment, the invention provides for the use of a compound of this invention in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the invention provides for the use of a compound of this invention in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the invention provides for the use of a compound of this invention in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the invention provides for the use of a compound of this invention in the therapeutic and/or prophylactic treatment of a disorder, disease, or disability associated with Kv7.2 in a subject in need thereof, the neurodegenerative diseases are selected from Alzheimer's disease and motor neuron diseases.

Further provided herein is the use of a a compound of this invention or a pharmaceutical composition comprising the same, for the manufacture of a medicament for the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

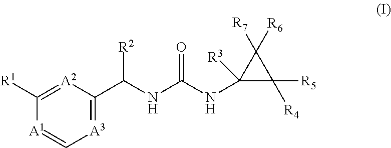

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy; or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
for the manufacture of a medicament for use in the therapeutic and/or prophylactic treatment of a disorder, disease, or disability associated with Kv7.2 in a subject in need thereof.

In one embodiment, the present invention provides the use of a compound of this invention, in particular of formulae (I)-(XI), or a solvate or a pharmaceutically acceptable salt thereof, any exemplified compound, any embodiment, or combinations of embodiments, as described herein, for the manufacture of a medicament for use in the therapeutic and/or prophylactic treatment of a disorder, disease, or disability associated with Kv7.2 in a subject in need thereof.

In one embodiment, the present invention provides the use a compound selected from Table 1, and 2, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound selected from any list of compounds described herein, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the mood disorder is depression.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the use of a compound of this invention for the manufacture of a medicament for use in the therapeutic and/or prophylactic treatment of a disorder, disease, or disability associated with Kv7.2 in a subject in need thereof, the neurodegenerative diseases are selected from Alzheimer's disease and motor neuron diseases.

Further provided a pharmaceutical composition comprising a compound as described herein, or solvate or a pharmaceutically acceptable salt thereof.

Further provided a pharmaceutical composition comprising a compound as described herein, or solvate or a pharmaceutically acceptable salt thereof, wherein the pharmaceutical composition further comprises one or more pharmaceutically acceptable excipients.

In one particularly preferred embodiment, the invention provides a pharmaceutical composition comprising a compound of formula (I), or a solvate or a pharmaceutically acceptable salt thereof:

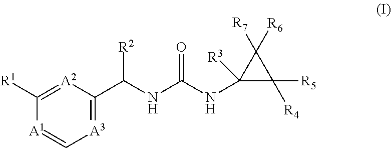

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one particularly preferred embodiment, the invention provides a pharmaceutical composition comprising a compound of formula (I), or a solvate or a pharmaceutically acceptable salt thereof:

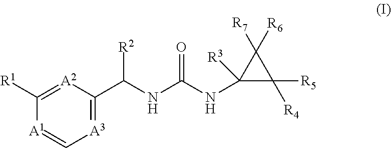

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy; or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

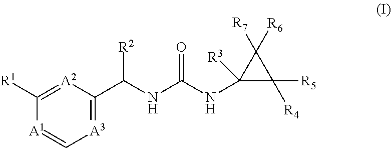

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, and haloC₁₋₆alkyl, wherein phenyl, or cycloalkyl are optionally substituted with one, two, or three substituents independently selected from halogen, haloC₁₋₆alkyl, and haloC₁₋₆alkoxy; and
R⁷ is selected from H, and C₁₋₆alkyl.

In one particularly preferred embodiment, the present invention provides a pharmaceutical composition comprising a compound of formula (I), or a solvate or a pharmaceutically acceptable salt thereof:

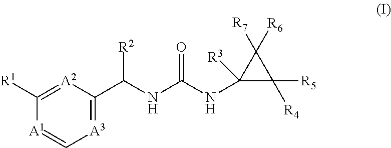

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkylm and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, and C₁₋₆alkyl;
and wherein the pharmaceutical composition further comprises one or more pharmaceutically acceptable excipients.

In one particularly preferred embodiment, the present invention provides a pharmaceutical composition comprising a compound of this invention, in particular of formulae (I)-(XI), or a solvate or a pharmaceutically acceptable salt thereof, as described herein, for use in the therapeutic and/or prophylactic treatment of a disorder, disease, or disability associated with Kv7.2 in a subject in need thereof.

In one particularly prefered embodiment, the present invention provides a pharmaceutical composition comprising a compound of formulae (I)-(XI), or a solvate or a pharmaceutically acceptable salt thereof, as described herein, for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one particularly preferred embodiment, the present invention provides a pharmaceutical composition comprising a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound of formulae (I)-(XI), or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, and 2, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound of this invention for use in a method of the therapeutic and/or prophylactic treatment of disorder, disease or disability associated with Kv7.2 in a subject in need thereof.

Further provided a pharmaceutical composition comprising a compound of this invention, and one or more pharmaceutically acceptable excipients, for use in a method of the therapeutic and/or prophylactic treatment of disorder, disease or disability associated with Kv7.2 in a subject in need thereof.

In one embodiment, the invention provides a pharmaceutical composition for use in a method of treatment comprising a compound of formula (I), or a solvate or a pharmaceutically acceptable salt thereof:

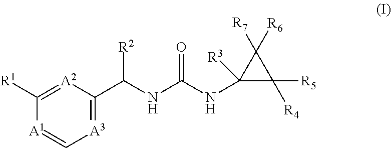

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a pharmaceutical composition for use in a method of treatment comprising a compound of formula (I), or a solvate or a pharmaceutically acceptable salt thereof:

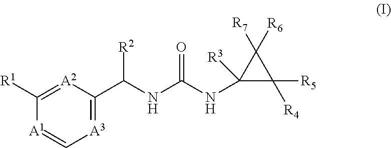

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

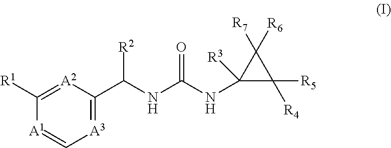

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the present invention provides a pharmaceutical composition for use in a method of treatment comprising a compound of formula (I), or a solvate or a pharmaceutically acceptable salt thereof:

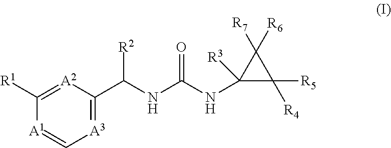

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition for use in a method of treatment comprises a compound of this invention for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the mood disorder is depression.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I)-(XI), or a solvate or a pharmaceutically acceptable salt thereof, any exemplified compound, any embodiment, or combinations of embodiments, as described herein.

In one embodiment, the present invention provides a pharmaceutical composition as described above comprising a compound selected from Table 1, and 2, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease, or disability associated with Kv7.2 in a subject in need thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease or disability associated with Kv7.2 in a subject in need thereof, wherein the pharmaceutical composition further comprises one or more pharmaceutically acceptable excipients.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

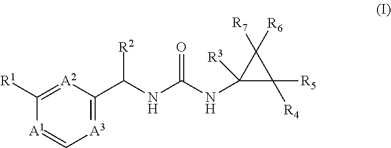

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

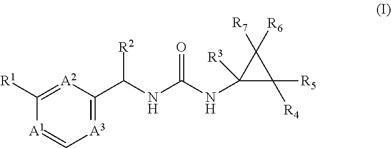

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkyl, C₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

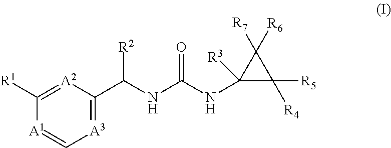

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

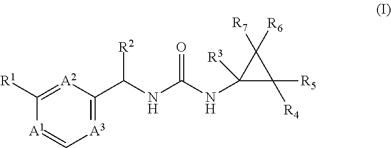

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

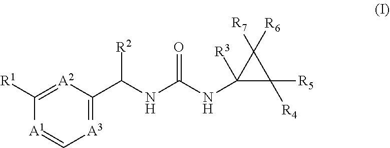

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

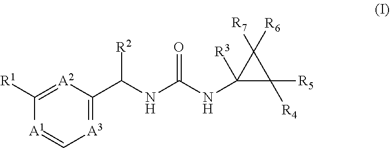

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition a compound of this invention for use in manufacture of a medicament for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I)-(XI), or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, and 2, or a solvate or a pharmaceutically acceptable salt thereof.

In one embodiment, the pharmaceutical composition as described above comprises a compound selected from any list of compounds described herein, or a solvate or a pharmaceutically acceptable salt thereof.

Any of the aforementioned embodiments in this section relating to medical uses, methods of treatment, and compounds for use in treatments may instead of a compound of formula (I) or other formula comprise or relate to a compound of formula (I′) or formula (II′) as described herein.

In addition to the options defined in this section medical uses, methods of treatment, and compounds for use in treatment, the compound of formula (I) may also comprise compounds as provided under the paragraphs Additional Options for Variables, Additional Options for R¹, Additional Options for R², and Formula (I′) as defined herein.

### Combination Therapy

Compounds of the invention may be combined with one or more other compounds of the invention or one or more other therapeutic agent as any combination thereof, in the treatment of the diseases, disorders, or disabilities provided herein. For example, a compound of the invention may be administered simultaneously, sequentially or separately in combination with other therapeutic agents known to be useful for the treatment of a disease or disorder selected from those recited herein.

In some embodiments, compounds of the invention may be combined with another therapeutically active agent having a synergistic effect in the treatment of any diseases, disorders, or disabilities described herein.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Articles of Manufacture

In one embodiment, the present invention provides an article of manufacture, or “kit”, containing materials useful for the treatment of the disorder, disease, or disability described herein is provided.

In one embodiment of the invention, the kit comprises a container comprising a compound of this invention, as described in any embodiment of this invention.

In one embodiment, the present invention provides a kit comprising a container comprising a compound of this invention, or a pharmaceutical composition thereof, as described herein.

In one embodiment, the present invention provides a kit comprising a compound of this invention, any exemplified compound, any embodiment, or combinations of embodiments, or pharmaceutical composition thereof, as described herein.

In one embodiment, the present invention provides a kit, wherein the compound is selected from formulae (I)-(XI), or a solvate, a pharmaceutically acceptable salt, or a pharmaceutical composition thereof, as described herein.

In one embodiment, the present invention provides a kit, wherein the compound is selected from Table 1, and 2, a solvate, a pharmaceutically acceptable salt, or a pharmaceutical composition thereof, as described herein.

In one embodiment, the present invention provides a kit for use in the treatment of a disorder, disease, or disability associated with Kv7.2, comprising:
a) a first pharmaceutical composition comprising a compound of this invention; and
b) instructions for use.

In one embodiment, the present invention provides a kit for use in the therapeutic and/or prophylactic treatment of a disorder, disease, or disability associated with Kv7.2 comprising:
a) a compound of this invention, or a pharmaceutical composition, or a pharmaceutical composition for use thereof; as described herein; and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising:
a) a first pharmaceutical composition comprising a compound of formula (I), or a solvate or a pharmaceutically acceptable salt, a pharmaceutical composition, or a pharmaceutical composition for use; as described herein:

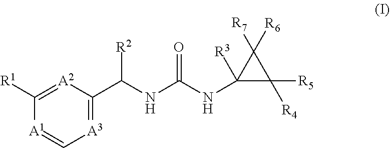

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆ alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkylalkoxy, or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen;
and
b) instructions for use.

In one embodiment, the present invention provides a kit for use in the therapeutic and/or prophylactic treatment of a disorder, disease, or disability associated with Kv7.2. These disorders, diseases or disabilities can be selected from behavioral disorders, mood disorders, neurodevelopmental disorders, intellectual disability, epilepsies, neurodegenerative diseases, pain, migraine, and tinnitus, comprising a compound, a pharmaceutical composition, or a pharmaceutical composition for use thereof, as described herein.

In one embodiment, the present invention provides a kit for use as described herein, wherein the behavioral disorder Attention Deficit Hyperactivity Disorder (ADHD).

In one embodiment, the present invention provides a kit for use as described herein, wherein the mood disorder is depression.

In one embodiment, the present invention provides a kit for use as described herein, wherein the neurodevelopment disorder is selected from autism spectrum disorder (ASD) and syndromic developmental disorders.

In one embodiment, the present invention provides a kit for use as described herein, wherein the syndromic developmental disorder is selected from Dup15q syndrome (Dup15q), Fragile X syndrome (FXS), and Angelman syndrome.

In one embodiment, the present invention provides a kit for use as described herein, wherein the epilepsies are selected from broad pediatric epilepsy, West syndrome, Ohtahara syndrome, and epileptic encephalopathy.

In one embodiment, the present invention provides a kit for use as described herein, wherein the neurodegenerative diseases are selected from Alzheimer's disease, and motor neuron diseases.

In one embodiment, the present invention provides a kit for use as described herein, wherein the kit further comprises a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products.

Suitable containers include, e.g., bottles, vials, syringes, blister pack, etc. The container may be formed from a variety of materials such as glass or plastic.

The container may hold a compound of this invention or a formulation thereof which is effective for treating the condition and may have a sterile access port (e.g., the container may be an intravenous solution bag or a vial having a stopper pierceable by a hypodermic injection needle). At least one active agent in the composition is a compound of this invention. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of this invention can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit further comprises directions for the administration of the compounds of this invention and, if present, the second pharmaceutical formulation. For example, if the kit comprises a first composition comprising a compound of this invention, and a second pharmaceutical formulation, the kit may further comprise directions for the simultaneous, sequential or separate administration of the first and second pharmaceutical compositions to a patient in need thereof.

In one embodiment, the present invention provides a kit for use as described herein, wherein the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

In one embodiment, the present invention provides a kit for use as described herein, wherein the kit comprises (a) a first container with a compound of this invention contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit comprises a composition of this invention and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Any of the aforementioned embodiments in this section relating to articles of manufacture may instead of a compound of formula (I) or other formula comprise a compound of formula (I′) or formula (II′) as described herein.

In addition to the options defined in this section articles of manufacture, the compound of formula (I) may also comprise compounds as provided under the paragraphs Additional Options for Variables, Additional Options for R¹, Additional Options for R², and Formula (I′) as defined herein.

### Methods of Manufacturing

In a further aspect, the present invention provides a compound of formula (I′) or (I), or a solvate or a pharmaceutically acceptable salt, when manufactured according to a process described herein.

The preparation of compounds of this invention, in particular compounds of formulae (I)-(XI), (I′) and (II′) may be carried out in sequential or convergent synthetic routes.

Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

In any of the embodiments relating to schemes or examples described in this application relating to a compound of this invention, any embodiment can be combined with any other embodiment unless contradictory.

If one of the starting materials, intermediates or compounds of this invention, in particular compounds of formula (I)-(XI), (I′) and (II′), contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5^{th} Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of this invention, in particular compounds of formula (I)-(XI), (I′) and (II′) can be obtained as mixtures of diastereomers or enantiomers, which can be separated by methods well known in the art e.g., chiral HPLC, chiral SFC, or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of this invention will typically lead to the respective diastereomerically/enantiomerically enriched compounds of this invention.

A person skilled in the art will acknowledge that in the synthesis of compounds of this invention, in particular compounds of formula (I)-(XI), (I′) and (II′) insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of this invention, i.e. compounds selected from formulae (I)-(XI) etc., or their solvates or pharmaceutically acceptable salts, can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis is not described in literature, they can be prepared in analogy to existing procedures for close analogues or as outlined in the experimental section.

### Abbreviations

EtOAc is ethyl acetate
CDI is 1,1′-carbonyldiimidazole
DCM is dichloromethane
DIPEA is N,N-diisopropylethylamine
DMA is Dimethylacetamide
DMF is N,N-dimethylformamide
HCl is hydrogen chloride
HPLC is high pressure liquid chromatography
LCMS is liquid chromatography mass spectrometry
mCPBA is 3-chloroperoxybenzoic acid
NaHCO₃ is sodium hydrogen carbonate
NaOH is sodium hydroxide
NMP is N-Methylpyrrolidone
MeOH is methanol
MgSO₄ is magnesium sulfate
Pd(dppf)Cl₂ is 1,1′-bis(di-tert-butylphosphino)ferrocene Palladium dichloride
PYBROP is bromotripyrrolidinophosphonium hexafluorophosphate
o/n is overnight
RT is room temperature
TLC is thin-layer chromatography
CHO is chinese hamster ovary
CMV is cytomegalovirus
FBS is fetal bovine serum
NEAA is non-essential amino acids
NaCl is sodium chloride
KCl is potassium chloride
CaCl₂ is calcium chloride
MgCl₂ is magnesium chloride
HEPES is 4-(2-hydroxyethyl)-1-piperazine ethanesulfonic acid
NMDG is N-methyl-D-glucamine diatrizoate
EGTA is ethylene glycol-bis(O-aminoethyl ether)-N,N,N′,N′-tetraacetic acid
EDTA is ethylenediaminetetraacetic acid
DPBS is Dulbecco's phosphate-buffered saline
mV is millivolt
TEA is tetraethylammonium
NADPH is nicotinamide adenine dinucleotide phosphate
CLᵢₙₜ is intrinsic clearance

The present invention provides compounds of formula (I), or a solvate or a pharmaceutically acceptable salts thereof:

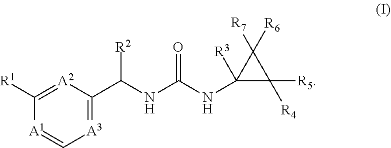

The preparation of compounds of formula (I), or other compounds of this invention may be carried out by reacting an amine with 1,1′-carbonyldiimidazole followed by addition of the second amine in-situ, or by first reacting an amine with para-nitro-phenyl chloroformate or phenyl chloroformate to yield the corresponding carbamate intermediate, which can be purified or used in situ by addition of a second amine. Syntheses of the compounds of the invention are shown in the following Schemes and in the description of 49 specific examples. The skills required for carrying out the reaction and purification of the resulting products are known to those skilled in the art.

In more detail, the compounds this invention, e.g. compounds selected from formula (I)-(XI), (I′) or (II′) or solvates or pharmaceutically acceptable salts thereof can be manufactured by the methods given in the examples or by analogous methods. Starting materials are either commercially available or can be prepared by methods analogous to the methods given below or by methods known in the art.

### General Procedures

For more information on the general procedures, please refer to the embodiments relating to the process of making compounds of this invention, in particular compounds of formula (I′), as described herein.

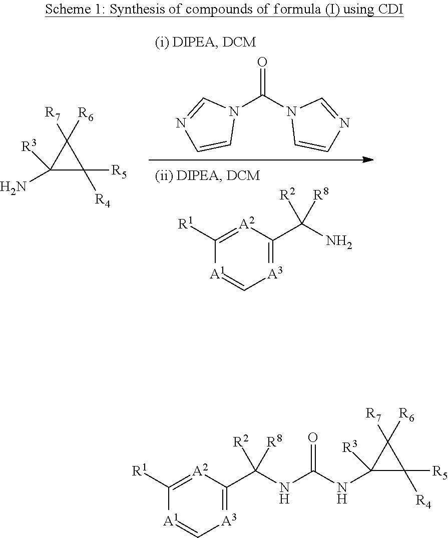

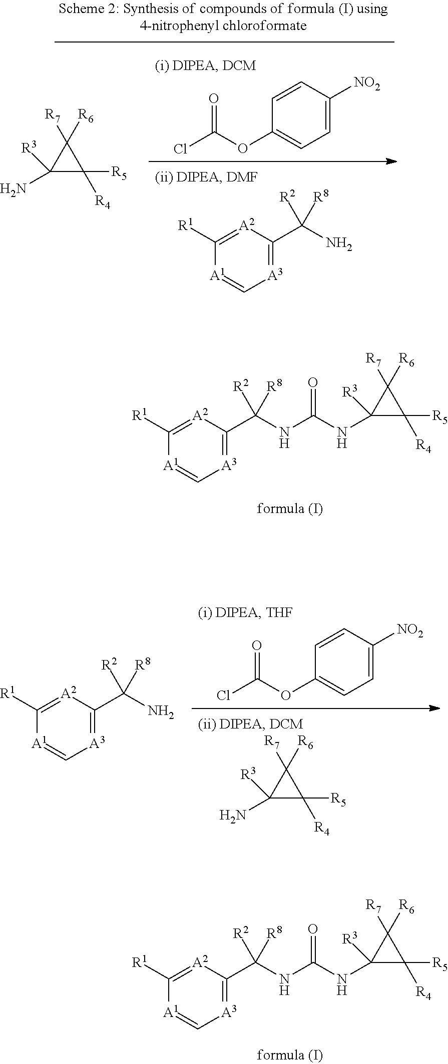

wherein R¹, R², R³, R⁴, R⁵, R⁶, R⁷, and R⁸ are as defined herein.

The urea formation can be accomplished by reacting the first amine with 1,1′-carbonyldiimidazole (Scheme 1) in a solvent (DCM, AcN, THF) and in the presence of a suitable base (DIPEA, NEt₃) to generate the activated urea prior to the addition of the second amine (or the corresponding salt), or by reacting the first amine with para-nitrophenyl chloroformate (Scheme 2) or phenyl chloroformate in a solvent (DCM, THF) and in the presence of a base (DIPEA, NEt₃), to generate the carbamate which can be purified or used in situ with a second amine to yield the desired urea.

Preferred conditions are using CDI with DIPEA as a base and with DCM as a solvent at 0° C. for 45 min and then adding the second amine and stirring at 35° C. for 2-6 hours.

Urea of formula I can be prepared by Suzuki coupling between a 2-bromo-pyridine and a boronic acid or ester using a Palladium catalyst like Pd(dppf)Cl₂ in the presence of a base like potassium carbonate in a solvent like acetonitrile (Scheme 3), either on the Boc protected amine or urea. Alternatively, a 2-bromo-pyridine can be reacted with a Bromo-oxetane in the presence of pyridine-2,6-dicarboximidamide dihydrochloride, Nickel(II) iodide, Sodium Iodide, Zinc and trifluoroacetic acid in a solvent like DMA to give a 2-(oxetan-3-yl)pyridine-based product.

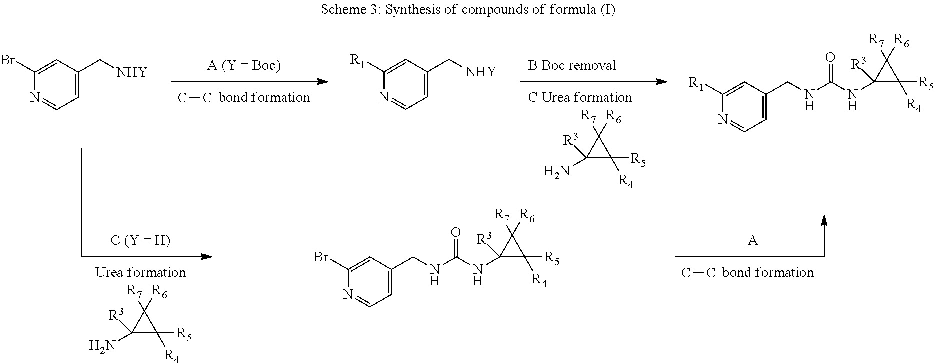

Urea of formula (I) (R² is H) can be prepared by reacting a pyridine with mCPBA in a solvent like DCM to generate an N-oxide intermediate (either as a di-Boc protected amine or urea), which can then be reacted with a nucleophile like a substituted imidazole or phenol in the presence of PYBROP and a base like NaHCO₃ in a solvent like DCM (Scheme 4).

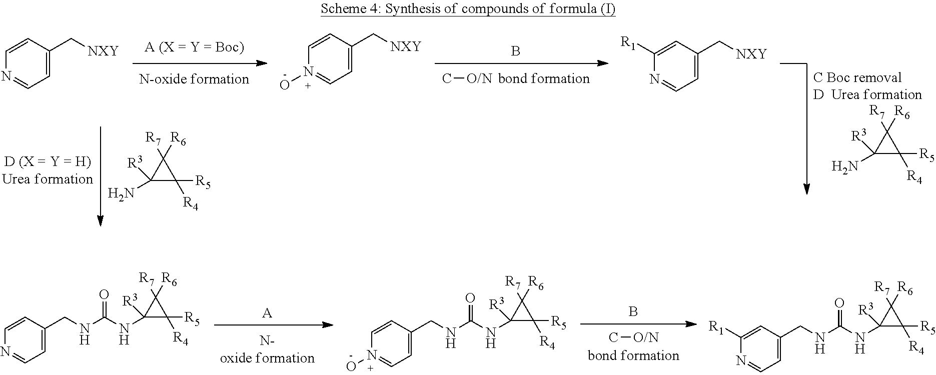

A pyridinone can be transformed to its corresponding 2-(difluoromethoxy)pyridine (Scheme 5) by reaction with sodium chlorodifluoroacetate in the presence of a base like potassium carbonate and in a solvent like NMP. Alternatively, a pyridinone can be reacted with 2-((fluoromethyl)(p-tolyl)-14-sulfaneylidene)malonate in the presence of a base like cesium carbonate and in a solvent like DMF to generate a 2-(fluoromethoxy)pyridine X

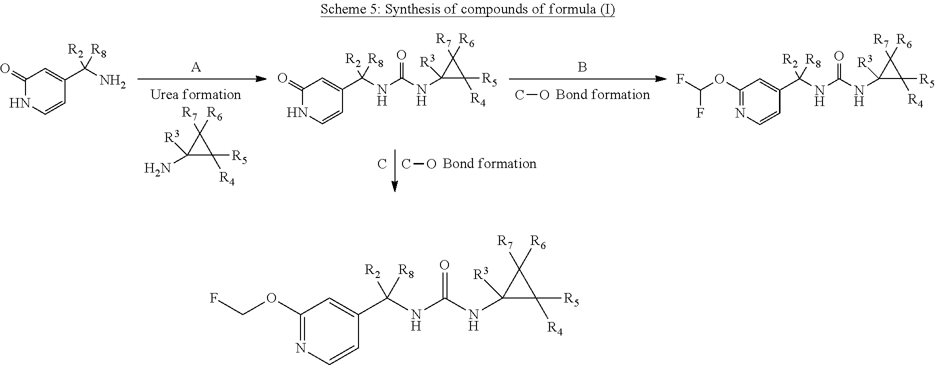

For more information on the general procedures, please refer to the embodiments relating to the process of making compounds of this invention, in particular compounds of formula (I)-(XI), as described herein.

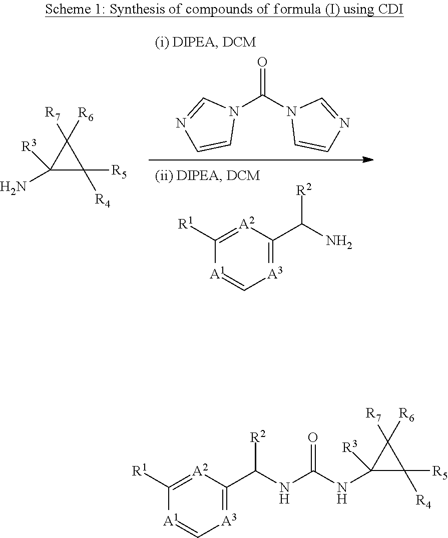

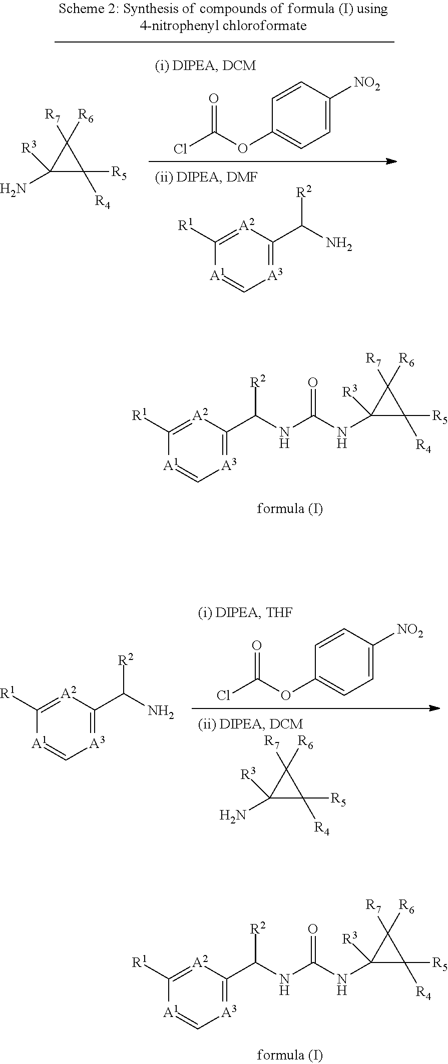

wherein R¹, R², R³, R⁴, R⁵, R⁶ and R⁷ are as defined herein.

The urea formation can be accomplished by reacting the first amine with 1,1′-carbonyldiimidazole (Scheme 1) in a solvent (DCM, AcN, THF) and in the presence of a suitable base (DIPEA, NEt₃) to generate the activated urea prior to the addition of the second amine (or the corresponding salt), or by reacting the first amine with para-nitrophenyl chloroformate (Scheme 2) or phenyl chloroformate in a solvent (DCM, THF) and in the presence of a base (DIPEA, NEt₃), to generate the carbamate which can be purified or used in situ with a second amine to yield the desired urea.

Preferred conditions are using CDI with DIPEA as a base and with DCM as a solvent at 0° C. for 45 min and then adding the second amine and stirring at 35° C. for 2-6 hours.

Urea of formula I can be prepared by Suzuki coupling between a 2-bromo-pyridine and a boronic acid or ester using a Palladium catalyst like Pd(dppf)Cl₂ in the presence of a base like potassium carbonate in a solvent like acetonitrile (Scheme 3), either on the Boc protected amine or urea. Alternatively, a 2-bromo-pyridine can be reacted with a Bromo-oxetane in the presence of pyridine-2,6-dicarboximidamide dihydrochloride, Nickel(II) iodide, Sodium Iodide, Zinc and trifluoroacetic acid in a solvent like DMA to give a 2-(oxetan-3-yl)pyridine-based product.

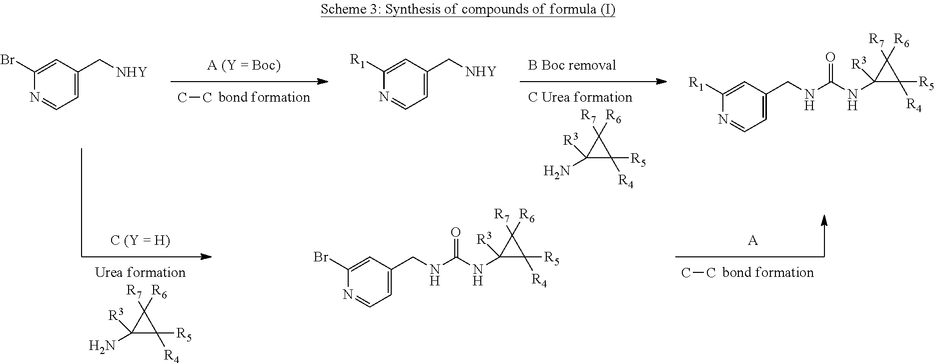

Urea of formula (I) (R² is H) can be prepared by reacting a pyridine with mCPBA in a solvent like DCM to generate an N-oxide intermediate (either as a di-Boc protected amine or urea), which can then be reacted with a nucleophile like a substituted imidazole or phenol in the presence of PYBROP and a base like NaHCO₃ in a solvent like DCM (Scheme 4).

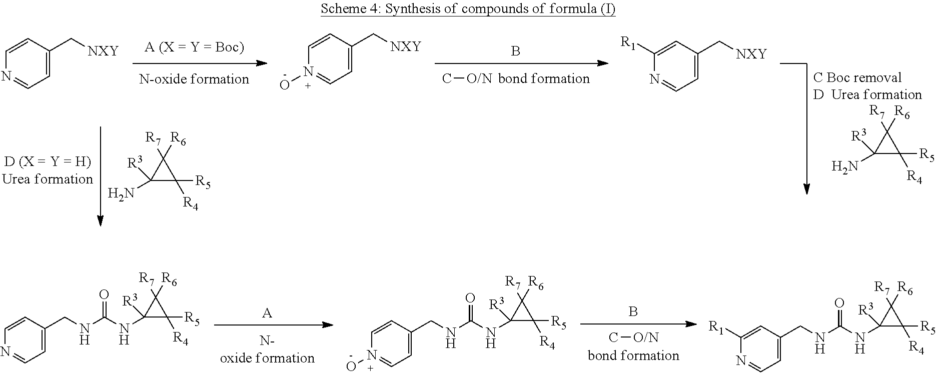

A pyridinone can be transformed to its corresponding 2-(difluoromethoxy)pyridine (Scheme 5) by reaction with sodium chlorodifluoroacetate in the presence of a base like potassium carbonate and in a solvent like NMP. Alternatively, a pyridinone can be reacted with 2-((fluoromethyl)(p-tolyl)-14-sulfaneylidene)malonate in the presence of a base like cesium carbonate and in a solvent like DMF to generate a 2-(fluoromethoxy)pyridine X

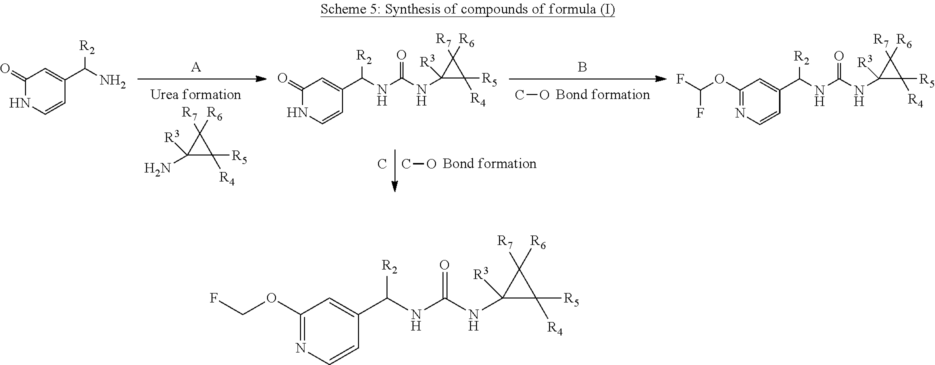

### Isolation and Purification of the Compounds

Isolation and purification of the compounds and intermediates described herein can be effected, if desired, by any suitable separation or purification procedure such as, for example, filtration, extraction, crystallization, column chromatography, thick-layer chromatography, preparative low or high-pressure liquid chromatography or a combination of these procedures. However, other equivalent separation or isolation procedures could, of course, also be used. Mixture of chiral compounds of formula (I) can be separated using preparative chiral HPLC purifications. Chiral HPLC purifications were performed on an AccQPrep HP125 (Teledyne ISCO) system, with a 5 μm 250 mm×21.2 mm i.d. chiral column (Amylose-1, Cellulose-1, or Cellulose-4) from Phenomenex, running at a flow rate of 20.8 mL min-1 with UV (214 and 254 nm) and ELS detection. Eluents: water; acetonitrile.

### Example 1: 1-[(4-cyanopyridin-2-yl)methyl]-3-[(1R,2S)-2-phenylcyclopropyl]urea

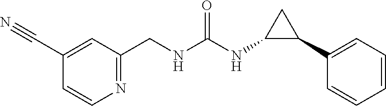 [SMILES: N#Cc1ccnc(CNC(=O)N[C@@H]2C[C@H]2c2ccccc2)c1]

### a) 4-nitrophenyl ((1R,2S)-2-phenylcyclopropyl)carbamate

(1R,2S)-2-phenylcyclopropan-1-amine hydrochloride (500 mg, 2.95 mmol) was stirred in dry DCM (15 mL) with DIPEA (1.13 mL, 6.48 mmol) and cooled to 0° C. under N₂. To this was then slowly added a solution of 4-nitrophenyl chloroformate (623.8 mg, 3.1 mmol) in dry DCM (7.5 mL) and the reaction was stirred at 0° C. for 1 h under N₂ then at RT for 1 h. The reaction was diluted with 40 ml DCM and washed with 30 ml sat NaHCO₃. The aqueous phase was extracted with further 40 ml DCM and the combined organics were washed with 2×30 ml sat NaHCO₃, 20 ml sat brine, dried over MgSO₄ and concentrated in vacuo. The crude was dry-loaded onto silica and purified by flash chromatography to yield 4-nitrophenyl ((1R,2S)-2-phenylcyclopropyl)carbamate (519 mg, 59%).

### b) 1-[(4-cyanopyridin-2-yl)methyl]-3-[(1R,2S)-2-phenylcyclopropyl]urea

To a solution of 4-(aminomethyl)picolinonitrile (35 mg, 0.264 mmol) in DMF (1.5 ml) and DIPEA (92 μl, 0.53 mmol) was added 4-nitrophenyl ((1R,2S)-2-phenylcyclopropyl)carbamate (75 mg, 0.25 mmol). The reaction was stirred at RT for 2 hrs. 1 equivalent of the amine with 5 equivalent of DIPEA was added, the reaction was complete after 2 hrs. Purification by preparative HPLC afforded the title product as a white solid (64% yield).

MS (m/z): 293 [M+H]+.

### Example 2: 1-[rac-(1R,2S)-2-cyclohexylcyclopropyl]-3-[(2-pyrazol-1-ylpyridin-4-yl)methyl]urea

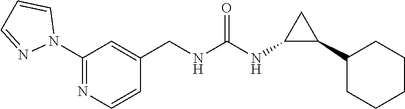 [SMILES: O=C(NCc1ccnc(-n2cccn2)c1)N[C@@H]1C[C@H]1C1CCCCC1]

To a solution of (3-(1H-pyrazol-1-yl)phenyl)methanamine (15 mg, 0.087 mmol) in DCM (0.5 ml) in an ice bath was added DIPEA (15 μl, 0.087 mmol) and phenyl chloroformate (13.6 mg, 0.087 mmol). The reaction was stirred at 0° C. for 1 hr, then a solution of rac-(1R,2S)-2-cyclohexylcyclopropan-1-amine hydrochloride (15 mg, 0.087 mmol) and DIPEA (15 μL, 0.087 mmol) in DCM (0.5 ml) was added. The reaction was stirred at 35° C. for 2 hr. The solvent was removed in vacuo, DMF (2 ml) was added and purification by preparative HPLC afforded the title compound as a white solid (41% yield).

MS (m/z): 340.1 [M+H]+.

### Example 3: 1-[(6-chloropyridin-2-yl)methyl]-3-[rac-(1R,2S)-2-cyclohexylcyclopropyl]urea

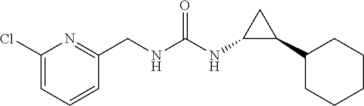 [SMILES: O=C(NCc1cccc(Cl)n1)N[C@@H]1C[C@H]1C1CCCCC1]

The title compound was obtained in analogy to Example 2 as a white solid (30% yield) using (6-chloropyridin-2-yl)methanamine, rac-(1R,2S)-2-cyclohexylcyclopropan-1-amine and phenyl chloroformate.

MS (m/z): 308.6 [M+H]+.

### Example 4: 1-[(2-propan-2-ylpyrimidin-4-yl)methyl]-3-[rac-(1R,2S)-2-cyclohexylcyclopropyl]urea

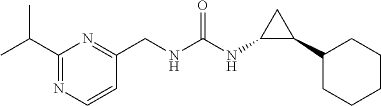 [SMILES: CC(C)c1nccc(CNC(=O)N[C@@H]2C[C@H]2C2CCCCC2)n1]

The title compound was obtained in analogy to Example 2 as a white solid (43% yield) using (2-isopropylpyrimidin-4-yl)methanamine, rac-(1R,2S)-2-cyclohexylcyclopropan-1-amine and phenyl chloroformate.

MS (m/z): 317.6 [M+H]+.

### Example 5: 1-[(2-methoxypyridin-4-yl)methyl]-3-[rac-(1R,2S)-2-phenylcyclopropyl]urea

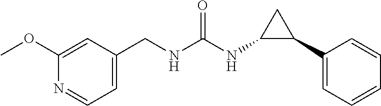 [SMILES: COc1cc(CNC(=O)N[C@@H]2C[C@H]2c2ccccc2)ccn1]

The title compound was obtained in analogy to Example 2 as a white solid (53% yield) using (2-methoxypyridin-4-yl)methanamine, rac-(1R,2S)-2-phenylcyclopropan-1-amine and phenyl chloroformate.

MS (m/z): 298.1 [M+H]+.

### Example 6: 1-[(1R,2S)-2-phenylcyclopropyl]-3-[(2-propan-2-yloxypyridin-4-yl)methyl]urea

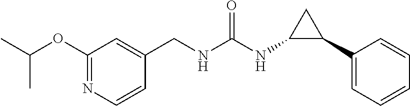 [SMILES: CC(C)Oc1cc(CNC(=O)N[C@@H]2C[C@H]2c2ccccc2)ccn1]

The title compound was obtained in analogy to Example 7 as a white solid (44% yield) using (2-isopropoxypyridin-4-yl)methanamine, (1R,2S)-2-phenylcyclopropan-1-amine and N,N′-carbonyldiimidazole.

MS (m/z): 326.2 [M+H]+.

### Example 7: 1-[[2-(difluoromethoxy)pyridin-4-yl]methyl]-3-[(1R,2S)-2-phenylcyclopropyl]urea

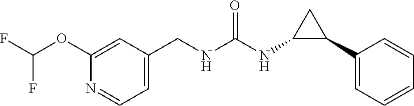 [SMILES: O=C(NCc1ccnc(OC(F)F)c1)N[C@@H]1C[C@H]1c1ccccc1]

To a solution of (1R,2S)-2-phenylcyclopropan-1-amine (20 mg, 0.12 mmol) in DCM (0.5 ml) in an ice batch was added DIPEA (40 μl, 0.24 mmol) and N,N′-carbonyldiimidazole (19.1 mg, 0.12 mmol). The reaction was stirred at 0° C. for 45 min, then (2-(difluoromethoxy)pyridin-4-yl)methanamine (20.5 mg, 0.12 mmol) was added. The reaction was stirred at 35° C. for 15 hrs. The solvent was removed in vacuo. DMF (2 ml) was added and purification by preparative HPLC afforded the title compound as a white solid (39 mg, 64% yield).

MS (m/z): 334.2 [M+H]+.

### Example 8: 1-[(2-cyanopyridin-4-yl)methyl]-3-[rac-(1R,2S)-2-phenylcyclopropyl]urea

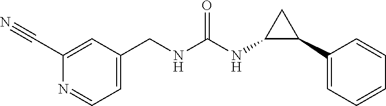 [SMILES: N#Cc1cc(CNC(=O)N[C@@H]2C[C@H]2c2ccccc2)ccn1]

The title compound was obtained in analogy to Example 2 as a white solid (46% yield) using 4-(aminomethyl)picolinonitrile, rac-(1R,2S)-2-phenylcyclopropan-1-amine and phenyl chloroformate.

MS (m/z): 293.3 [M+H]+.

### Example 9: 1-[(2-methoxypyridin-4-yl)methyl]-3-[(1R,2S)-2-phenylcyclopropyl]urea

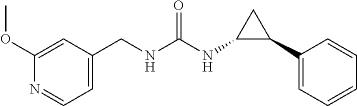 [SMILES: COc1cc(CNC(=O)N[C@@H]2C[C@H]2c2ccccc2)ccn1]

The title compound was obtained in analogy to Example 7 as a white solid (60% yield) using (2-methoxypyridin-4-yl)methanamine, (1R,2S)-2-phenylcyclopropan-1-amine and N,N′-carbonyldiimidazole.

MS (m/z): 298.2 [M+H]+.

### Example 10: 1-[(2-cyanopyridin-4-yl)methyl]-3-[(1R,2S)-2-phenylcyclopropyl]urea

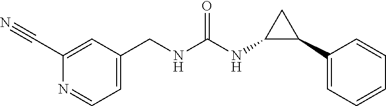 [SMILES: N#Cc1cc(CNC(=O)N[C@@H]2C[C@H]2c2ccccc2)ccn1]

The title compound was obtained in analogy to Example 2 as a white solid (50% yield) using 4-(aminomethyl)picolinonitrile, (1R,2S)-2-phenylcyclopropan-1-amine and phenyl chloroformate.

MS (m/z): 293.3 [M+H]+.

### Example 11: 1-(2-cyclohexylcyclopropyl)-3-[(2-imidazol-1-ylpyridin-4-yl)methyl]urea

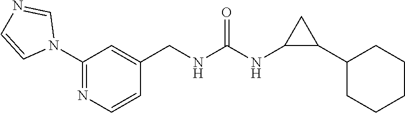 [SMILES: O=C(NCc1ccnc(-n2ccnc2)c1)NC1CC1C1CCCCC1]

The compound can be prepared by methods similar to those described herein.

MS (m/z): 340.1 [M+H]+.

### Example 12: 1-[(2-pyrazol-1-ylpyridin-4-yl)methyl]-3-[rac-(1R,2S)-2-phenylcyclopropyl]urea

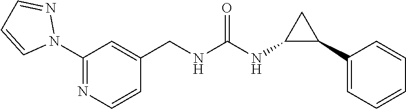 [SMILES: O=C(NCc1ccnc(-n2cccn2)c1)N[C@@H]1C[C@H]1c1ccccc1]

The title compound was obtained in analogy to Example 2 as a white solid (37% yield) using (2-(1H-pyrazol-1-yl)pyridin-4-yl)methanamine, rac-(1R,2S)-2-phenylcyclopropan-1-amine and phenyl chloroformate.

MS (m/z): 334.5 [M+H]+.

### Example 13: 1-[(1R,2S)-2-phenylcyclopropyl]-3-[(2-pyrazol-1-ylpyridin-4-yl)methyl]urea

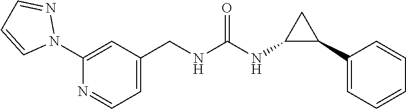 [SMILES: O=C(NCc1ccnc(-n2cccn2)c1)N[C@@H]1C[C@H]1c1ccccc1]

The title compound was obtained in analogy to Example 7 as a white solid (69% yield) using (2-(1H-pyrazol-1-yl)pyridin-4-yl)methanamine, (1R,2S)-2-phenylcyclopropan-1-amine and N,N′-carbonyldiimidazole.

MS (m/z): 334.4 [M+H]+.

### Example 14: 1-[(1R,2S)-2-phenylcyclopropyl]-3-[[2-(2,2,2-trifluoroethoxy)pyridin-4-yl]methyl]urea

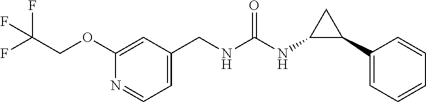 [SMILES: O=C(NCc1ccnc(OCC(F)(F)F)c1)N[C@@H]1C[C@H]1c1ccccc1]

The title compound was obtained in analogy to Example 7 as a white solid (70% yield) using (2-(2,2,2-trifluoroethoxy)pyridin-4-yl)methanamine, (1R,2S)-2-phenylcyclopropan-1-amine and N,N′-carbonyldiimidazole.

MS (m/z): 366.5 [M+H]+.

### Example 15: 1-[(1S,2R)-2-cyclohexylcyclopropyl]-3-[(2-imidazol-1-ylpyridin-4-yl)methyl]urea

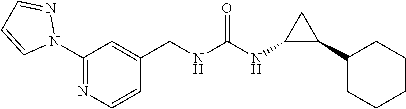 [SMILES: O=C(NCc1ccnc(-n2cccn2)c1)N[C@@H]1C[C@H]1C1CCCCC1]

The title compound was obtained by chiral HPLC separation of Example 11 (114 mg, 38% yield) as a white solid (Amylose 1 column in 40:60 water:MeCN).

MS (m/z): 340.3 [M+H]+.

### Example 16: 1-[[2-(oxetan-3-yloxy)pyridin-4-yl]methyl]-3-[(1R,2S)-2-phenylcyclopropyl]urea

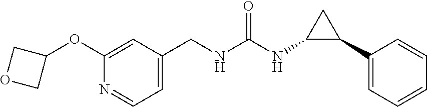 [SMILES: O=C(NCc1ccnc(OC2COC2)c1)N[C@@H]1C[C@H]1c1ccccc1]

The title compound was obtained in analogy to Example 7 as a white solid (55% yield) using (2-(oxetan-3-yloxy)pyridin-4-yl)methanamine, (1R,2S)-2-phenylcyclopropan-1-amine and N,N′-carbonyldiimidazole.

MS (m/z): 340.2 [M+H]+.

### Example 17: 1-[(2-chloropyridin-4-yl)methyl]-3-[rac-(1R,2S)-2-cyclohexylcyclopropyl]urea

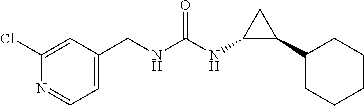 [SMILES: O=C(NCc1ccnc(Cl)c1)N[C@@H]1C[C@H]1C1CCCCC1]

The title compound was obtained in analogy to Example 7 as a white solid (45% yield) using (2-chloropyridin-4-yl)methanamine, rac-(1R,2S)-2-cyclohexylcyclopropan-1-amine and N,N′-carbonyldiimidazole.

MS (m/z): 308.2 [M+H]+.

### Example 18: 1-[(6-cyanopyridin-2-yl)methyl]-3-[(1R,2S)-2-phenylcyclopropyl]urea

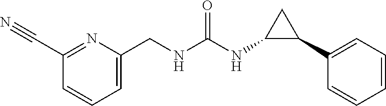 [SMILES: N#Cc1cccc(CNC(=O)N[C@@H]2C[C@H]2c2ccccc2)n1]

The title compound was obtained in analogy to Example 1 as a white solid (60% yield) using 6-(aminomethyl)picolinonitrile, (1R,2S)-2-phenylcyclopropan-1-amine and 4-nitrophenyl chloroformate.

MS (m/z): 293.3 [M+H]+.

### Example 19: 1-[(2-chloropyridin-4-yl)methyl]-3-[(1R,2S)-2-phenylcyclopropyl]urea

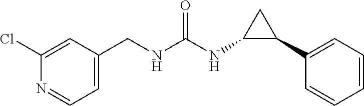 [SMILES: O=C(NCc1ccnc(Cl)c1)N[C@@H]1C[C@H]1c1ccccc1]

The title compound was obtained in analogy to Example 1 as a white solid (28% yield) using (2-chloropyridin-4-yl)methanamine and (1R,2S)-2-phenylcyclopropan-1-amine and 4-nitrophenyl chloroformate.

MS (m/z): 302.3 [M+H]+.

### Example 20: 1-[[2-(oxan-4-yloxy)pyridin-4-yl]methyl]-3-[(1R,2S)-2-phenylcyclopropyl]urea

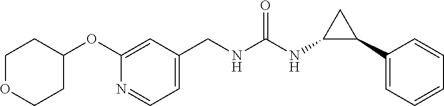 [SMILES: O=C(NCc1ccnc(OC2CCOCC2)c1)N[C@@H]1C[C@H]1c1ccccc1]

The title compound was obtained in analogy to Example 7 as a white solid (42% yield) using (2-((tetrahydro-2H-pyran-4-yl)oxy)pyridin-4-yl)methanamine, (1R,2S)-2-phenylcyclopropan-1-amine and N,N′-carbonyldiimidazole.

MS (m/z): 368.3 [M+H]+.

### Example 21: 1-[[2-(difluoromethyl)pyridin-4-yl]methyl]-3-[(1R,2S)-2-phenylcyclopropyl]urea

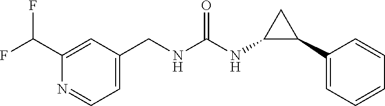 [SMILES: O=C(NCc1ccnc(C(F)F)c1)N[C@@H]1C[C@H]1c1ccccc1]

The title compound was obtained in analogy to Example 7 as a white solid (61% yield) using (2-(difluoromethyl)pyridin-4-yl)methanamine, (1R,2S)-2-phenylcyclopropan-1-amine and N,N′-carbonyldiimidazole.

MS (m/z): 318.3 [M+H]+.

### Example 22: 1-[(1R,2S)-2-phenylcyclopropyl]-3-[[2-(trifluoromethyl)pyridin-4-yl]methyl]urea

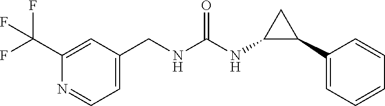 [SMILES: O=C(NCc1ccnc(C(F)(F)F)c1)N[C@@H]1C[C@H]1c1ccccc1]

The title compound was obtained in analogy to Example 7 as a white solid (78% yield) (2-(trifluoromethyl)pyridin-4-yl)methanamine, (1R,2S)-2-phenylcyclopropan-1-amine and N,N′-carbonyldiimidazole.

MS (m/z): 336.3 [M+H]+.

### Example 23: 1-[[2-(1,3-oxazol-5-yl)pyridin-4-yl]methyl]-3-[rac-(1R,2S)-2-cyclohexylcyclopropyl]urea

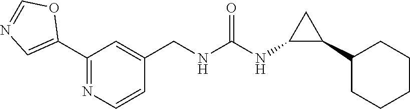 [SMILES: O=C(NCc1ccnc(-c2cnco2)c1)N[C@@H]1C[C@H]1C1CCCCC1]

### a) tert-butyl N-{[2-(1,3-oxazol-5-yl)pyridin-4-yl]methyl}carbamate

To a mixture of tert-butyl-N-[(2-bromopyridin-4-yl)methyl]carbamate (100 mg, 0.35 mmol, 1 eq), Oxazole-5-boronic acid pinacol ester (74.7 mg, 0.38 mmol, 1.1 eq), potassium carbonate (96.3 mg, 0.7 mmol, 2 eq) in Acetonitrile (1.5 mL) and Water (1.5 mL) under N₂ was added 1,1′-bis(di-tert-butylphosphino)ferrocene Palladium dichloride (11.35 mg, 0.02 mmol, 0.05 eq). The reaction mixture was sparged with N₂ again for 5 min and then heated under N₂ at 80° C. for 2 hr, then allowed to cool to RT and left o/n. The mixture was partitioned between EtOAc (25 ml) and water (15 ml). The organic layer was washed with brine (20 ml), dried (MgSO₄) and evaporated in vacuo to afford a crude brown oil (130 mg), which was purified by flash chromatography on silica eluting with a Heptane-60% EtOAc/Heptane gradient to yield tert-butyl N-{[2-(1,3-oxazol-5-yl)pyridin-4-yl]methyl}carbamate (94 mg, 0.29 mmol, 97%) as a white solid.

MS (m/z): 176 [M-Boc+H]+.

### b) [2-(1,3-oxazol-5-yl)pyridin-4-yl]methanamine

To a solution of tert-butyl N-{[2-(1,3-oxazol-5-yl)pyridin-4-yl]methyl}carbamate (90 mg, 0.33 mmol, 1 eq) in Ethyl Acetate (2 mL) under N₂ was added HCl (4M in Dioxane) (2.04 mL (4M), 8.17 mmol, 25 eq) slowly. The reaction was stirred at RT under N₂ o/n. The reaction mixture was cooled in an ice bath and the white precipitate filtered off and washed with cold ether. The collected solid was dried under high vacuum to afford [2-(1,3-oxazol-5-yl)pyridin-4-yl]methanamine dihydrochloride as an off-white solid (73 mg, 90%), which was taken forward without further purification.

MS (m/z): 176 [M+H]+.

### c) 1-[[2-(1,3-oxazol-5-yl)pyridin-4-yl]methyl]-3-[rac-(1R,2S)-2-cyclohexylcyclopropyl]urea

The title compound was obtained in analogy to Example 7 as a white solid (33% yield) using [2-(1,3-oxazol-5-yl)pyridin-4-yl]methanamine dihydrochloride, rac-(1R,2S)-2-cyclohexylcyclopropan-1-amine hydrochloride and N,N′-carbonyldiimidazole.

MS (m/z): 341 [M+H]+.

### Example 24: 1-[rac-(1R,2S)-2-cyclohexylcyclopropyl]-3-[[2-[4-(trifluoromethyl)imidazol-1-yl]pyridin-4-yl]methyl]urea

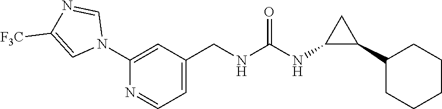 [SMILES: O=C(NCc1ccnc(-n2cnc(C(F)(F)F)c2)c1)N[C@@H]1C[C@H]1C1CCCCC1]

### a) 4-((bis(tert-butoxycarbonyl)amino)methyl)pyridine 1-oxide

Tert-butyl (tert-butoxycarbonyl)(pyridin-4-ylmethyl)carbamate (1 g, 3.24 mmol, 1 eq) and 3-chloroperoxybenzoic acid (2.14 mL (77%), 0.56 g/mL, 5.35 mmol, 1.65 eq) were mixed in a flask and DCM (35 mL) was added. The reaction was complete after minutes by TLC and 1 N NaOH sol. (40 ml) and DCM (40 ml) were added. The aqueous phase was extracted with DCM (2×40 ml). The combined organic phase was dried (MgSO₄) and evaporated in vacuo to afford 4-((bis(tert-butoxycarbonyl)amino)methyl)pyridine 1-oxide as a yellow oil (1.1 g, 3.79 mmol, 100%).

MS (m/z): 325 [M+H]+.

### b) tert-butyl N-(tert-butoxycarbonyl)-N-({2-[4-(trifluoromethyl)imidazol-1-yl]pyridin-4-yl}methyl)carbamate

To an N₂ flushed flask containing 4-((bis(tert-butoxycarbonyl)amino)methyl)pyridine 1-oxide (400 mg, 1.23 mmol, 1 eq), DCM (6 mL) was added followed by DIPEA (0.81 mL, 4.62 mmol, 3.75 eq) and 4-(Trifluoromethyl)-1H-imidazole (218.14 mg, 1.6 mmol, 1.3 eq). The reaction mixture was sonicated to get the imidazole into solution. PYBROP (747.3 mg, 1.6 mmol, 1.3 eq) was added and left to stir at RT o/n. DCM (30 ml) and Sat. NaHCO₃ sol. (25 ml) were added and partitioned. The organic layer was dried (MgSO₄) and evaporated in vacuo to afford a crude pale yellow oil (1.35 g). Purification by flash chromatography on silica eluting with Heptane-32% EtOAc/Heptane (gradient) affored the desired tert-butyl N-(tert-butoxycarbonyl)-N-({2-[4-(trifluoromethyl)imidazol-1-yl]pyridin-4-yl}methyl)carbamate (463 mg, 1.05 mmol, 85%).

MS (m/z): 443 [M+H]+.

### c) (2-(4-(trifluoromethyl)-1H-imidazol-1-yl)pyridin-4-yl)methanamine

To a solution of tert-butyl N-(tert-butoxycarbonyl)-N-({2-[4-(trifluoromethyl)imidazol-1-yl]pyridin-4-yl}methyl)carbamate (460 mg, 1.04 mmol, 1 eq) in Ethyl Acetate (4 mL) under N₂, HCl (4M in Dioxane) (4.16 mL (4M), 16.64 mmol, 16 eq) was added slowly. The reaction was stirred at RT under N₂ o/n. The reaction mixture was cooled in an ice bath and filtered off and washed with cold ether. The white precipitate was collected and dried under vacuum to afford (2-(4-(trifluoromethyl)-1H-imidazol-1-yl)pyridin-4-yl)methanamine as an off-white solid (264 mg, 0.84 mmol, 80.7%).

MS (m/z): 243 [M+H]+.

### d) 1-[rac-(1R,2S)-2-cyclohexylcyclopropyl]-3-[[2-[4-(trifluoromethyl)imidazol-1-yl]pyridin-4-yl]methyl]urea

The title compound was obtained in analogy to Example 7 as a white solid (64% yield) using (2-(4-(trifluoromethyl)-1H-imidazol-1-yl)pyridin-4-yl)methanamine, rac-(1R,2S)-2-cyclohexylcyclopropan-1-amine and N,N′-carbonyldiimidazole.

MS (m/z): 408 [M+H]+.

### Example 25: 1-[(2-cyclobutyloxypyridin-4-yl)methyl]-3-[(1R,2S)-2-phenylcyclopropyl]urea

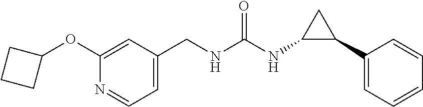 [SMILES: O=C(NCc1ccnc(OC2CCC2)c1)N[C@@H]1C[C@H]1c1ccccc1]

The title compound was obtained in analogy to Example 7 as a white solid (32% yield) using (2-cyclobutoxypyridin-4-yl)methanamine, (1R,2S)-2-phenylcyclopropan-1-amine and N,N′-carbonyldiimidazole.

MS (m/z): 338.4 [M+H]+.

### Example 26: 1-[[2-(1,1-difluoroethyl)pyridin-4-yl]methyl]-3-[(1R,2S)-2-phenylcyclopropyl]urea

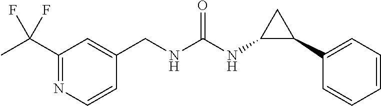 [SMILES: CC(F)(F)c1cc(CNC(=O)N[C@@H]2C[C@H]2c2ccccc2)ccn1]

The title compound was obtained in analogy to Example 1 as a white solid (72% yield) using (2-(1,1-difluoroethyl) pyridin-4-yl) methanamine, (1R,2S)-2-phenylcyclopropan-1-amine and 4-nitrophenyl chloroformate.

MS (m/z): 332 [M+H]+.

### Example 27: 1-[(2-imidazol-1-ylpyridin-4-yl)methyl]-3-[(1R,2S)-2-phenylcyclopropyl]urea

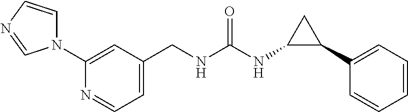 [SMILES: O=C(NCc1ccnc(-n2ccnc2)c1)N[C@@H]1C[C@H]1c1ccccc1]

The title compound was obtained in analogy to Example 1 as a white solid (70% yield) using (2-(1H-imidazol-1-yl)pyridin-4-yl)methanamine, (1R,2S)-2-phenylcyclopropan-1-amine and 4-nitrophenyl chloroformate.

MS (m/z): 334 [M+H]+.

### Example 28: 1-[(1R,2S)-2-phenylcyclopropyl]-3-[[2-(trifluoromethoxy)pyridin-4-yl]methyl]urea

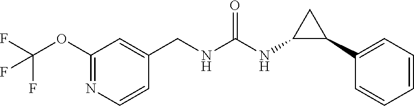 [SMILES: O=C(NCc1ccnc(OC(F)(F)F)c1)N[C@@H]1C[C@H]1c1ccccc1]

The title compound was obtained in analogy to Example 1 as a white solid (84% yield) using (2-(trifluoromethoxy)pyridin-4-yl)methanamine, (1R,2S)-2-phenylcyclopropan-1-amine and 4-nitrophenyl chloroformate.

MS (m/z): 352 [M+H]+.

### Example 29: 1-[(1S)-1-(2-methoxypyridin-4-yl)ethyl]-3-[(1R,2S)-2-phenylcyclopropyl]urea

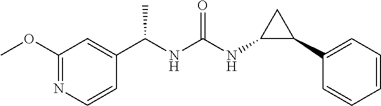 [SMILES: COc1cc([C@H](C)NC(=O)N[C@@H]2C[C@H]2c2ccccc2)ccn1]

The title compound was obtained in analogy to Example 1 as a white solid (68% yield) using (S)-1-(3-methoxyphenyl)ethan-1-amine, (1R,2S)-2-phenylcyclopropan-1-amine and 4-nitrophenyl chloroformate.

MS (m/z): 312 [M+H]+.

### Example 30: 1-((S)-1-(2-(difluoromethoxy)pyridin-4-yl)ethyl)-3-((1R,2S)-2-phenylcyclopropyl)urea

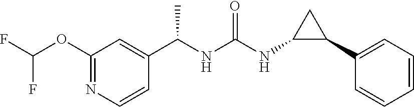 [SMILES: C[C@H](NC(=O)N[C@@H]1C[C@H]1c1ccccc1)c1ccnc(OC(F)F)c1]

### a) 1-(1-(2-oxo-1,2-dihydropyridin-4-yl)ethyl)-3-((1R,2S)-2-phenylcyclopropyl)urea

The title compound was obtained in analogy to Example 1 as a white solid (84% yield) using 4-(1-aminoethyl)pyridin-2-ol, (1R,2S)-2-phenylcyclopropan-1-amine and 4-nitrophenyl chloroformate.

MS (m/z): 298.1 [M+H]+.

### b) 1-((S)-1-(2-(difluoromethoxy)pyridin-4-yl)ethyl)-3-((1R,2S)-2-phenylcyclopropyl)urea

To an oven-dried reaction vial, equipped with a magnetic stirrer and under nitrogen was added sodium chlorodifluoroacetate 97% (75.9 mg, 0.5 mmol) and potassium carbonate (68.8 mg, 0.5 mmol). A solution of 1-(1-(2-oxo-1,2-dihydropyridin-4-yl)ethyl)-3-((1R,2S)-2-phenylcyclopropyl)urea (98.7 mg, 0.33 mmol) in NMP (2 mL) was added, and the mixture was stirred at 75° C. for 6 hrs, then left to stir at 60° C. overnight. LCMS showed incomplete reaction. The reaction mixture was allowed to cool, then extra Sodium chlorodifluoroacetate (75.9 mg, 0.5 mmol), potassium carbonate (68.8 mg, 0.5 mmol), and NMP (1.5 mL) were added and the reaction was heated at 80° C. for 3 hrs. The reaction was allowed to cool, then concentrated in vacuo. The material was filtered (0.2 um PTFE) and the filter was rinsed through with DMSO (1 mL). The combined solution was directly injected and purified by preparative HPLC automated flash chromatography to yield 1-(1-(2-(difluoromethoxy)pyridin-4-yl)ethyl)-3-((1R,2S)-2-phenylcyclopropyl)urea (59 mg, 51% yield). Chiral HPLC separation (Amylose 1 column in 55:45 water:MeCN) afforded the title compound (22.3 mg, 37.9% yield) as a white solid, as well as its diastereomer (Example 38).

MS (m/z): 348.2 [M+H]+.

### Example 31: 1-[(1R,2S)-2-phenylcyclopropyl]-3-[[2-[4-(trifluoromethyl)imidazol-1-yl]pyridin-4-yl]methyl]urea

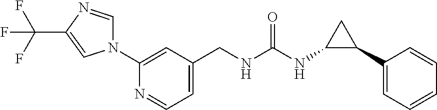 [SMILES: O=C(NCc1ccnc(-n2cnc(C(F)(F)F)c2)c1)N[C@@H]1C[C@H]1c1ccccc1]

### a) 1-((1R,2S)-2-phenylcyclopropyl)-3-(pyridin-4-ylmethyl)urea

The compound was obtained in analogy to Example 7 as a white solid (80% yield) using pyridin-4-ylmethanamine, (1R,2S)-2-phenylcyclopropan-1-amine and N,N′-carbonyldiimidazole.

MS (m/z): 268 [M+H]+.

### b) 4-((3-((1R,2S)-2-phenylcyclopropyl)ureido)methyl)pyridine 1-oxide

To a solution of 1-((1R,2S)-2-phenylcyclopropyl)-3-(pyridin-4-ylmethyl)urea (1 g, 3.74 mmol) in DCM (50 mL) was added 3-chloroperoxybenzoic acid (2.47 mL (77%), 0.56 g/mL, 6.17 mmol). The mixture immediately went into solution. The reaction was left to stir at RT o/n. DCM (50 ml) and 1N NaOH (50 ml) were added. The layers were separated and the aqeuous phase was reexttracted with DCM (6×100 ml+few mls of iPrOH). The combined organic phases were dried (MgSO4) and evaporated in vacuo to afford a crude oil (1.2 g). Purification by flash chromatography (silica) eluting with DCM −15% MeOH/DCM gradient afforded the desired product as a white foam (84.3%).

MS (m/z): 284 [M+H]+.

### c) 1-[(1R,2S)-2-phenylcyclopropyl]-3-[[2-[4-(trifluoromethyl)imidazol-1-yl]pyridin-4-yl]methyl]urea

To an N₂ flushed flask containing 4-((3-((1R,2S)-2-phenylcyclopropyl)ureido)methyl)pyridine 1-oxide (60 mg, 0.21 mmol, 1 eq) in DCM (2 mL) was added DIPEA (0.14 mL, 0.79 mmol, 3.75 eq) and 4-(Trifluoromethyl)-1H-imidazole (37.5 mg, 0.28 mmol, 1.3 eq). PYBROP (128.34 mg, 0.28 mmol, 1.3 eq) was then added and the reaction was left to stir at RT for 48 hrs. DCM (20 ml) and Sat. NaHCO₃ sol. (20 ml) were added and partitioned. The combined organic phases were dried (MgSO₄) and evaporated in vacuo to afford a crude oil (215 mg) which was applied to a silica column eluting with DCM-7% MeOH/DCM to afford the impure product (82 mg). The solid was dissolved in DMSO (2 ml) and purified by prep HPLC to yield the title product as a white solid (47 mg, 55% yield).

MS (m/z): 402 [M+H]+.

### Example 32: 1-[[2-(oxetan-3-yl)pyridin-4-yl]methyl]-3-[(1R,2S)-2-phenylcyclopropyl]urea

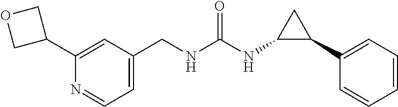 [SMILES: O=C(NCc1ccnc(C2COC2)c1)N[C@@H]1C[C@H]1c1ccccc1]

### a) 1-((2-bromopyridin-4-yl)methyl)-3-((1R,2S)-2-phenylcyclopropyl)urea

The title compound was obtained in analogy to Example 1 as a white solid (57% yield) using (2-bromopyridin-4-yl)methanamine di-hydrochloride, (1R,2S)-2-phenylcyclopropan-1-amine and 4-nitrophenyl chloroformate.

LCMS: m/z=346.1 [M+H].

### b) 1-[[2-(oxetan-3-yl)pyridin-4-yl]methyl]-3-[(1R,2S)-2-phenylcyclopropyl]urea

To an 8 mL vial was added sequentially DMA (1 mL), pyridine-2,6-dicarboximidamide dihydrochloride (1.77 mg, 7.51 mol, 0.05 eq), Nickel(II) iodide (2.35 mg, 7.51 mol, 0.05 eq), Sodium Iodide (5.63 mg, 0.04 mmol, 0.25 eq). To this was then added 1-[(2-bromopyridin-4-yl)methyl]-3-[(1R,2S)-2-phenylcyclopropyl]urea (52 mg, 0.15 mmol, 1 eq) dissolved in DMA (1 mL) followed by 3-Bromooxetane (24.92 μL, 1.65 g/mL, 0.3 mmol, 2 eq), Zinc (19.64 mg, 0.3 mmol, 2 eq) and finally trifluoroacetic acid (1.15 μL, 0.02 mmol, 0.1 eq). The vial was sealed with a septa-lid and the headspace purged with N₂. The reaction mixture was then agitated at 60 C on the heater/shaker overnight (after ˜10 mins Zn goes into solution). LMCS indicated only trace reaction. Additional reagents (same quantities as at start of reaction) with the exception of the urea SM were added to the vial again, the headspace purged and the reaction mixture heated at 60° C. on the heater/shaker for a further 18 hours. LCMS now showed product to be the major peak. The reaction mixture was diluted with EtOAc and filtered through a celite cartridge (2.5 g) washing with EtOAc. The filtrate was then washed with diluted aqueous ammonia solution. The aqueous was then back-extracted with EtOAc once, combined organics were dried over MgSO₄ and concentrated under reduced pressure to yield an orange oil. This was taken up in 0.5 ml of 10% MeOH/DMSO, filtered through a cotton wool plug and then purified by preparative HPLC eluting with 5 to 95% ACN/0.1% FA in Water/0.1% FA to yield the title product as a white powder (8.0% yield).

LCMS: m/z=324.2 [M+H].

### Example 33: 1-[(1R,2S)-2-phenylcyclopropyl]-3-[(1S)-1-[2-[4-(trifluoromethyl)imidazol-1-yl]pyridin-4-yl]ethyl]urea

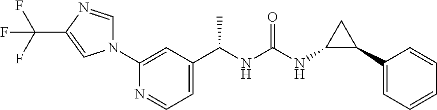 [SMILES: C[C@H](NC(=O)N[C@@H]1C[C@H]1c1ccccc1)c1ccnc(-n2cnc(C(F)(F)F)c2)c1]

The title compound was obtained in analogy to Example 31 as a white solid (81% yield) using 4-((S)-1-(3-((1R,2S)-2-phenylcyclopropyl)ureido) ethyl)pyridine 1-oxide, 4-(Trifluoromethyl)-1H-imidazole and PYBROP.

MS (m/z): 416 [M+H]+.

### Example 34: 1-[(2-phenoxypyridin-4-yl)methyl]-3-[(1R,2S)-2-phenylcyclopropyl]urea

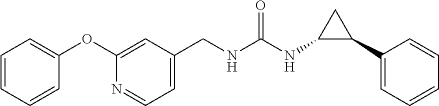 [SMILES: O=C(NCc1ccnc(Oc2ccccc2)c1)N[C@@H]1C[C@H]1c1ccccc1]

The title compound was obtained in analogy to Example 31 as a white solid (76% yield) using 4-((3-((1R,2S)-2-phenylcyclopropyl)ureido)methyl) pyridine 1-oxide, phenol and PYBROP.

MS (m/z): 360 [M+H]+.

### Example 35: 1-[[2-(2,2-difluoroethoxy)pyridin-4-yl]methyl]-3-[(1R,2S)-2-phenylcyclopropyl]urea

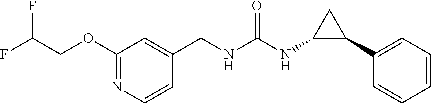 [SMILES: O=C(NCc1ccnc(OCC(F)F)c1)N[C@@H]1C[C@H]1c1ccccc1]

The title compound was obtained in analogy to example 1 as a white solid (43% yield) using (2-(2,2-difluoroethoxy)pyridin-4-yl)methanamine, (1R,2S)-2-phenylcyclopropan-1-amine and 4-nitrophenyl chloroformate.

MS (m/z): 348 [M+H]+.

### Example 36: 1-[[2-(difluoromethoxy)pyridin-4-yl]methyl]-3-[rac-(1R,2S)-1-methyl-2-phenylcyclopropyl]urea

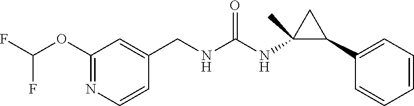 [SMILES: C[C@@]1(NC(=O)NCc2ccnc(OC(F)F)c2)C[C@H]1c1ccccc1]

The title compound was obtained in analogy to Example 1 as a white solid (27% yield) using (2-(difluoromethoxy)pyridin-4-yl)methanamine, rac-(1R,2S)-1-methyl-2-phenylcyclopropan-1-amine and 4-nitrophenyl chloroformate.

MS (m/z): 348 [M+H]+.

### Example 37: 1-[[2-(fluoromethoxy)pyridin-4-yl]methyl]-3-[(1R,2S)-2-phenylcyclopropyl]urea

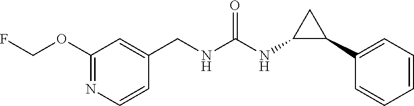 [SMILES: O=C(NCc1ccnc(OCF)c1)N[C@@H]1C[C@H]1c1ccccc1]

### a) 1-((2-hydroxypyridin-4-yl)methyl)-3-((1R,2S)-2-phenylcyclopropyl)urea

1-((2-hydroxypyridin-4-yl)methyl)-3-((1R,2S)-2-phenylcyclopropyl)urea was obtained in analogy to Example 1 as a white solid (74% yield) using 4-(aminomethyl)pyridin-2-ol hydrochloride, (1R,2S)-2-phenylcyclopropan-1-amine and 4-nitrophenyl chloroformate.

MS (m/z): 284 [M+H]+.

### b) 1-[[2-(fluoromethoxy)pyridin-4-yl]methyl]-3-[(1R,2S)-2-phenylcyclopropyl]urea

1-((2-hydroxypyridin-4-yl)methyl)-3-((1R,2S)-2-phenylcyclopropyl)urea (0.50 mmol, 1.0 equiv), cesium carbonate (325 mg, 1.0 mmol, 2.0 equiv), and the ylide dimethyl 2-((fluoromethyl)(p-tolyl)-14-sulfaneylidene)malonate⁽¹⁾ (272 mg, 1.0 mmol, 2.0 equiv) were placed into an oven-dried Schlenk tube that was equipped with a stirring bar under nitrogen. The tube was quickly sealed with a rubber stopper and 3.0 mL of freshly distilled DMF was added. The reaction was stirred at 40° C. for 30 min. Then the reaction mixture was cooled to room temperature. 20 mL of distilled water and 40 mL of Et₂O were added then the organic phase was separated.

The organic phase was washed with H₂O (5×20 mL) and the organic extracts were dried (Na₂SO₄) and concentrated in vacuo. The crude product was purified by flash chromatography on silica to yield the title product as a white solid (3.3% yield).

MS (m/z): 316 [M+H]+.

### Example 38: 1-[1-[2-(difluoromethoxy)pyridin-4-yl]ethyl]-3-[(1R,2S)-2-phenylcyclopropyl]urea

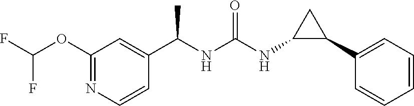 [SMILES: C[C@@H](NC(=O)N[C@@H]1C[C@H]1c1ccccc1)c1ccnc(OC(F)F)c1]

The title compound is the other diastereomer isolated by chiral separation of Example 30 (21.9 mg, 37.2% yield).

MS (m/z): 348.2 [M+H]+.

### Example 39: 1-[(1R,2S)-2-phenylcyclopropyl]-3-[[2-[3-(trifluoromethyl)pyrazol-1-yl]pyridin-4-yl]methyl]urea

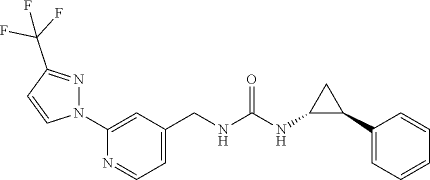 [SMILES: O=C(NCc1ccnc(-n2ccc(C(F)(F)F)n2)c1)N[C@@H]1C[C@H]1c1ccccc1]

The title compound was obtained in analogy to Example 31 as a white solid (56% yield) using 4-((3-((1R,2S)-2-phenylcyclopropyl)ureido)methyl)pyridine 1-oxide, 3-(trifluoromethyl)-1H-pyrazole and PYBROP.

MS (m/z): 402.2 [M+H]+.

### Example 40: 1-[rac-(1R,2S)-2-cyclohexylcyclopropyl]-3-[[2-(1,2-thiazol-4-yl)pyridin-4-yl]methyl]urea

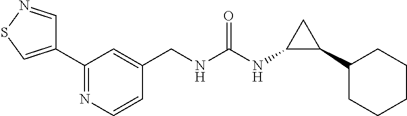 [SMILES: O=C(NCc1ccnc(-c2cnsc2)c1)N[C@@H]1C[C@H]1C1CCCCC1]

### a) 1-[(2-bromopyridin-4-yl)methyl]-3-[(1R,2S)-2-cyclohexylcyclopropyl]urea

The title compound was obtained in analogy to Example 1 as a yellowish solid (65% yield) using (2-bromopyridin-4-yl)methanamine, rac-(1R,2S)-2-cyclohexylcyclopropan-1-amine and 4-nitrophenyl chloroformate.

MS (m/z): 352 [M+H]+.

### b) 1-[rac-(1R,2S)-2-cyclohexylcyclopropyl]-3-[[2-(1,2-thiazol-4-yl)pyridin-4-yl]methyl]urea

A mixture of Isothiazole-4-boronic acid pinacol ester (32.0 mg, 0.15 mmol, 1 eq), 1-[(2-bromopyridin-4-yl)methyl]-3-[(1R,2S)-2-cyclohexylcyclopropyl]urea (80 mg, 0.23 mmol, 1.5 eq) and 1,1′-bis(di-tert-butylphosphino)ferrocene Palladium dichloride (4.94 mg, 0.01 mmol, 0.05 eq) in acetonitrile (1 mL) and water (0.1 mL) was made in a 8 mL microwave vial and degassed for 10 mins. The reaction was microvwave irradiated to 100° C. for 20 mins. The reaction was filtered through celite, and washed with EtOAc and concetrated. Crude was purified by preparative HPLC eluting with 5 to 95% ACN/0.1% FA in Water/0.1% FA to yield the title product as a peach colored solid (25% yield).

MS (m/z): 357.2 [M+H]+.

### Example 41: 1-[[2-(difluoromethoxy)pyridin-4-yl]methyl]-3-[(1R,2R)-2-(trifluoromethyl)cyclopropyl]urea

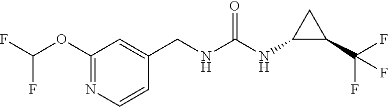 [SMILES: O=C(NCc1ccnc(OC(F)F)c1)N[C@@H]1C[C@H]1C(F)(F)F]

### a) 4-nitrophenyl N-{[2-(difluoromethoxy)pyridin4-yl]methyl}carbamate

A solution of 4-Nitrophenyl chloroformate (6.94 g, 34.45 mmol, 1.2 eq) in THF (130 mL) under N₂ was cooled to −5° C. To this was added dropwise a solution of 1-[2-(difluoromethoxy)pyridin-4-yl]methanamine (5 g, 28.71 mmol, 1 eq) and DIPEA (5 mL, 28.71 mmol, 1 eq) in THF (100 mL) over 45 min. After 5 min the cooling bath was removed to allow the reaction to warm to RT. After 2.5 h the reaction was complete by LCMS and concentrated in vacuo to afford a yellow oil, which was partitioned between EtOAc (2×120 ml) and NaHCO₃ sol (120 ml). The combined organic layers was dried (MgSO₄) and concentrated in vacuo to afford a crude yellow solid (11 g) which was purified by flash chromatography on Silica eluting with heptane-43% EtOAc/heptane (gradient) to afford 4-nitrophenyl N-{[2-(difluoromethoxy)pyridin4-yl]methyl}carbamate as a slightly impure pale yellow solid (8.17 g, 24.08 mmol, 83.9%).

### b) 1-[[2-(difluoromethoxy)pyridin-4-yl]methyl]-3-[(1R,2R)-2-(trifluoromethyl)cyclopropyl]urea

To a mixture of (1R,2R)-2-(Trifluoromethyl)cyclopropan-1-amine hydrochloride (48.5 mg, 0.3 mmol, 1 eq) in DCM (3 mL) was added DIPEA (0.1 mL, 0.6 mmol, 2 eq) followed by 4-nitrophenyl N-{[2-(difluoromethoxy)pyridin4-yl]methyl}carbamate (101.8 mg, 0.3 mmol, 1 eq). The reaction was stirred at RT o/n, then concentrated in vacuo to afford a crude oil, which was dissolved in DMSO (2 ml) and purified by preparative HPLC to afford the title product (75 mg, 0.23 mmol, 76.9% yield).

MS (m/z): 326.1 [M+H]+.

### Example 42: 1-[[2-(difluoromethoxy)pyridin-4-yl]methyl]-3-[rac-(1R,3R)-2,2-difluoro-3-methylcyclopropyl]urea

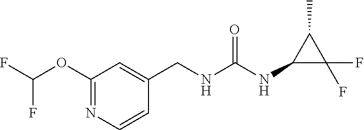 [SMILES: C[C@H]1[C@H](NC(=O)NCc2ccnc(OC(F)F)c2)C1(F)F]

The title compound was obtained in analogy to Example 7 as an off-white solid (66% yield) using (2-(difluoromethoxy)pyridin-4-yl)methanamine hydrochloride, rac-(1S,3S)-2,2-difluoro-3-methylcyclopropan-1-amine hydrochloride and N,N′-carbonyldiimidazole.

MS (m/z): 308 [M+H]+.

### Example 43: 1-[[2-(difluoromethoxy)pyridin-4-yl]methyl]-3-[rac-(1R,2R)-2-(oxolan-2-yl)cyclopropyl]urea

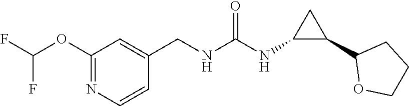 [SMILES: O=C(NCc1ccnc(OC(F)F)c1)N[C@@H]1C[C@H]1C1CCCO1]

The title compound was obtained in analogy to Example 41 as a white solid (28% yield) using (2-(difluoromethoxy)pyridin-4-yl)methanamine, rac-(1R,2R)-2-(oxolan-2-yl)cyclopropan-1-amine oxalic acid and 4-nitrophenyl chloroformate.

MS (m/z): 328.1 [M+H]+.

### Example 44: 1-[[2-(difluoromethoxy)pyridin-4-yl]methyl]-3-[rac-(1R,2R)-2-(1,1-difluoroethyl)cyclopropyl]urea

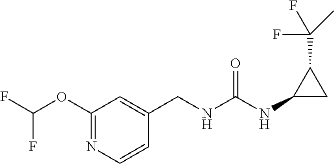 [SMILES: CC(F)(F)[C@@H]1C[C@H]1NC(=O)NCc1ccnc(OC(F)F)c1]

The title compound was obtained in analogy to Example 7 as a peach colored solid (47% yield) using (2-(difluoromethoxy)pyridin-4-yl)methanamine hydrochloride, rac-(1R,2R)-2-(1,1-difluoroethyl)cyclopropan-1-amine hydrochloride and N,N′-carbonyldiimidazole.

MS (m/z): 322.1 [M+H]+.

### Example 45: 1-(2-cyclohexylcyclopropyl)-3-[[2-(oxan-4-yloxy)pyridin-4-yl]methyl]urea

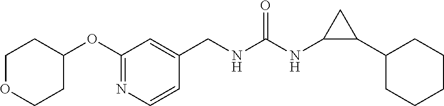 [SMILES: O=C(NCc1ccnc(OC2CCOCC2)c1)NC1CC1C1CCCCC1]

The compound can be prepared by methods similar to those described herein.

MS (m/z): 374.2 [M+H]+.

### Example 46: 1-[[2-(difluoromethoxy)pyridin-4-yl]methyl]-3-[rac-(1R,2R)-2-methylcyclopropyl]urea

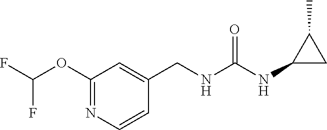 [SMILES: C[C@@H]1C[C@H]1NC(=O)NCc1ccnc(OC(F)F)c1]

The title compound was obtained in analogy to Example 7 as a peach colored solid (38% yield) using (2-(difluoromethoxy)pyridin-4-yl)methanamine hydrochloride, rac-(1R,2R)-2-methylcyclopropan-1-amine hydrochloride and N,N′-carbonyldiimidazole.

MS (m/z): 272.1 [M+H]+.

### Example 47: 1-[[2-(difluoromethoxy)pyridin-4-yl]methyl]-3-[rac-(1R,2R)-2-methyl-2-(trifluoromethyl)cyclopropyl]urea

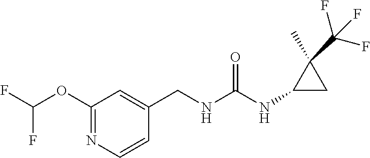 [SMILES: C[C@]1(C(F)(F)F)C[C@@H]1NC(=O)NCc1ccnc(OC(F)F)c1]

The title compound was obtained in analogy to Example 41 as an off-white solid (49% yield) using (2-(difluoromethoxy)pyridin-4-yl)methanamine, rac-(1R,2R)-2-methyl-2-trifluoromethyl)cyclopropan-1-amine hydrochloride and 4-nitrophenyl chloroformate.

MS (m/z): 340.1 [M+H]+.

### Example 48: 1-[[2-(2,3-difluoropropoxy)pyridin-4-yl]methyl]-3-[(1R,2S)-2-phenylcyclopropyl]urea

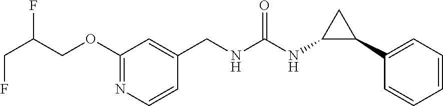 [SMILES: O=C(NCc1ccnc(OCC(F)CF)c1)N[C@@H]1C[C@H]1c1ccccc1]

### a) (2-(2,3-difluoropropoxy)pyridin-4-yl)methanamine

tert-Butyl-(2-chloropyridin-4-yl)methylcarbamate (1 g, 4.12 mmol, 1 eq), tBuBrettPhos Pd G3 (70.41 mg, 0.08 mmol, 0.02 eq) and cesium carbonate (2.01 g, 6.18 mmol, 1.5 eq) were combined in a dry 100 ml flask. This was flushed with nitrogen before adding degassed Toluene (15 mL) with 2,3-difluoropropan-1-ol (0.59 g, 6.18 mmol, 1.5 eq) as a solution. The resultant brown mixture was heated at 80° C. under N₂. After 3 h the reaction mixture was allowed to cool, diluted with CH2Cl2, and filtered through Celite. The filtrate was evaporated in vacuo to afford a crude solid (1.49 g), which was purified by flash chromatography on Silica eluting with heptane-25% EtOAc/heptane to afford tert-butyl N-{[2-(2,3-difluoropropoxy)pyridin-4-yl]methyl}carbamate as a yellow oil (1.05 g, 3.46 mmol, 84.1%). The product was then dissolved in Ethyl Acetate (15 mL) and HCl (4M in Dioxane) (17.2 mL (4M), 68.8 mmol, 20 eq) was added and the mixture stirred o/n. White precipitate had formed. The reaction was cooled in an ice bath and some cold ether was added. The precipitate was filtered off, washed with further cold ether and dried in vacuo to afford [2-(2,3-difluoropropoxy)pyridin-4-yl]methanamine dihydrochloride (825 mg, 87.2%) as a white solid.

MS (m/z): 203 [M+H]+.

### b) 1-[[2-(2,3-difluoropropoxy)pyridin-4-yl]methyl]-3-[(1R,2S)-2-phenylcyclopropyl]urea

The title compound was obtained in analogy to Example 41 as a white solid (55% yield) using (2-(2,3-difluoropropoxy)pyridin-4-yl)methanamine dihydrochloride, (1R,2S)-2-phenylcyclopropan-1-amine hydrochloride and 4-nitrophenyl chloroformate.

MS (m/z): 362.1 [M+H]+.

### Example 49: 1-[[2-(difluoromethoxy)pyrimidin-4-yl]methyl]-3-[(1R,2R)-2-(trifluoromethyl)cyclopropyl]urea

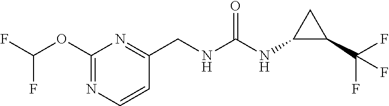 [SMILES: O=C(NCc1ccnc(OC(F)F)n1)N[C@@H]1C[C@H]1C(F)(F)F]

The title compound was obtained in analogy to Example 1 as a cream colored solid (36% yield) using (1R,2R)-2-(trifluoromethyl)cyclopropan-1-amine, [2-(difluoromethoxy)pyrimidin-4-yl]methanamine and 4-nitrophenyl chloroformate.

MS (m/z): 327.1 [M+H]+.

### Example 50: 1-[dideuterio-[2-(difluoromethoxy)pyridin-4-yl]methyl]-3-[rac-(1R,2R)-2-(trifluoromethyl)cyclopropyl]urea

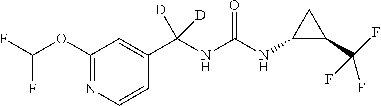 [SMILES: [2H]C([2H])(NC(=O)N[C@@H]1C[C@H]1C(F)(F)F)c1ccnc(OC(F)F)c1]

### a) tert-butyl N-{[2-(difluoromethoxy)pyridin-4-yl](²H₂)methyl}carbamate

Nickel (II) Chloride, dimethoxyethane adduct (49.0 mg, 0.22 mmol) was added to a degassed solution of 4-bromo-2-(difluoromethoxy)pyridine (1 g, 4.46 mmol), Boc-Gly-OH-2,2-d2 (1186.6 mg, 6.7 mmol), (4,4′-Di-tbutyl-2,2′-bipyridine)bis[3,5-difluoro-2-[5-trifluoromethyl-2-pyridinyl-kN)phenyl-kC]iridium(III) hexafluorophosphate, 99% (50.1 mg, 0.04 mmol) and 4,4′-di-tert-butyl-2,2′-dipyridyl (59.9 mg, 0.22 mmol) in dry DMF (40 mL). The solution was then split into two 40 mL vials containing cesium carbonate (1.09 g, 3.35 mmol). The vials were irradiated under a blue LED (450 nm, 54 W at 60% intensity) at 22° C. for 2 h. The reaction was then diluted in water and product was extracted with EtOAc, and organic layers were combined, washed with 5% LiCl (aq) and brine, dried with MgSO₄, filtered and concentrated. The crude was purified by silica gel chromatography (eluting with 0 to 65% Ethyl Acetate in Heptane) to give the product (280 mg, 1.01 mmol, 22.7% yield) as an off-white solid.

MS (m/z): 277.2 [M+H]+.

### b) (2-(difluoromethoxy)pyridin-4-yl)(²H₂)methanamine

HCl (4M in Dioxane) (2.53 mL, 10.13 mmol) was added to a solution of tert-butyl N-{[2-(difluoromethoxy)pyridin-4-yl](²H2)methyl}carbamate (280 mg, 1.01 mmol) in Ethyl Acetate (10 mL) and stirred at rt overnight. Another HCl (4M in Dioxane) (2.53 mL, 10.13 mmol) was added and stirred for 3 h. HCl was further preciptated with cold ether and then filtered under vaccum. The salt was then washed with more cold ether and left to dry under vacuum for 20 mins to yield the product as the hydrochloride salt (160 mg, 0.75 mmol, 74.3% yield) as a white solid.

MS (m/z): 177.2 [M+H]+.

### c) 1-[dideuterio-[2-(difluoromethoxy)pyridin-4-yl]methyl]-3-[rac-(1R,2R)-2-(trifluoromethyl)cyclopropyl]urea

The title compound was obtained in analogy to Example 7 as a white solid (59.6% yield) using rac-(1R,2R)-2-(trifluoromethyl)cyclopropan1-amine hydrochloride, (2-(difluoromethoxy)pyridin-4-yl)(²H)methanamine hydrochloride and N,N′-carbonyldiimidazole.

MS (m/z): 328.1 [M+H]+.

### Example 51: 1-[rac-(1R,2R)-2-(trifluoromethyl)cyclopropyl]-3-[[6-(2,2,2-trifluoroethoxy)pyrimidin-4-yl]methyl]urea

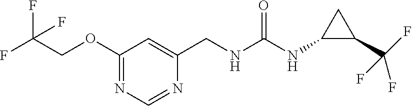 [SMILES: O=C(NCc1cc(OCC(F)(F)F)ncn1)N[C@@H]1C[C@H]1C(F)(F)F]

The title compound was obtained in analogy to Example 1 as a white solid (50.6% yield) using [6-(2,2,2-trifluoroethoxy)pyrimidin-4-yl]methanamine hydrochloride, rac-(1R,2R)-2-(trifluoromethyl)cyclopropan1-amine hydrochloride and 4-nitrophenyl chloroformate.

MS (m/z): 359.1 [M+H]+.

### Example 52: 1-[[6-(difluoromethoxy)pyrimidin-4-yl]methyl]-3-[(1S,2S)-2-(trifluoromethyl)cyclopropyl]urea

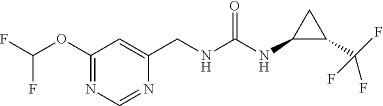 [SMILES: O=C(NCc1cc(OC(F)F)ncn1)N[C@H]1C[C@@H]1C(F)(F)F]

### a) 4-(difluoromethoxy)-6-methylpyrimidine

6-Methyl-3,4-dihydropyrimidin-4-one (1 g, 9.08 mmol) and sodium chloro(difluoro)acetate (1.66 g, 10.9 mmol) in Acetonitrile (50 mL) was purged under N₂ and then heated at 90° C. for 32 h. The reaction was cooled to room temperature then filtered through celite, and washed with pentane, the filtrate was concentrated and the crude purified by silica gel chromatography (eluting with 0 to 100% Dichloromethane in Pentane) to yield the product (152.5 mg, 0.95 mmol, 10.5% yield) as a cream solid.

MS (m/z): 161 [M+H]+.

### b) 4-(bromomethyl)-6-(difluoromethoxy)pyrimidine

To a solution of 4-(difluoromethoxy)-6-methylpyrimidine (312 mg, 1.95 mmol) in carbon tetrachloride (15 mL) was added N-bromosuccinimide (346.8 mg, 1.95 mmol) and benzoyl peroxide (0.02 mL, 1.16 g/mL, 0.1 mmol). The reaction was further sparged with nitrogen, then heated to relfux at 80° C. for 16 h. The reaction was thereafter permitted to cool to rt. The reaction was diluted with dichloromethane and the crude was adsorbed on to isolute for purification by silica gel chromatography (eluting with 0-20% ethyl acetate in heptane) to yield the product (44.34 mg, 0.19 mmol, 9.5% yield) as a beige solid.

### c) (6-(difluoromethoxy)pyrimidin-4-yl)methanamine

4-(bromomethyl)-6-(difluoromethoxy)pyrimidine (44 mg, 0.18 mmol) was dissolved in ammonia (0.15 mL, 1.02 g/mL, 7M, 9.2 mmol) in a 30 mL microwave vial. This was microwaved at 80° C. for 10 min. The excess ammonia was removed under reduced pressure, then co-evaporated with EtOAc (3×5 mL) to give crude product as cream solid (35.86 mg, 0.18 mmol, 99% yield).

MS (m/z): 176 [M+H]+.

### d) 1-[[6-(difluoromethoxy)pyrimidin-4-yl]methyl]-3-[(1S,2S)-2-(trifluoromethyl)cyclopropyl]urea

The title compound was obtained in analogy to Example 7 as a cream colored solid (40.7% yield) using (1S,2S)-2-(trifluoromethyl)cyclopropan-1-amine hydrochloride, (6-(difluoromethoxy)pyrimidin-4-yl)methanamine and N,N′-carbonyldiimidazole.

MS (m/z): 327.1 [M+H]+.

### Example 53: 1-[[2-[(2R)-1,1,1-trifluoro-3-methoxypropan-2-yl]oxypyridin-4-yl]methyl]-3-[rac-(1R,2R)-2-(trifluoromethyl)cyclopropyl]urea

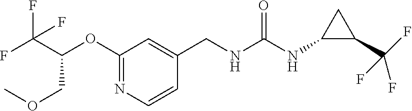 [SMILES: COC[C@@H](Oc1cc(CNC(=O)N[C@@H]2C[C@H]2C(F)(F)F)ccn1)C(F)(F)F]

### a) 2-[(1,1,1-trifluoro-3-methoxypropan-2-yl)oxy]pyridine-4-carbonitrile

To an ice cooled solution of 1,1,1-trifluoro-3-methoxypropan-2-ol (623.98 mg, 4.33 mmol) in DMF (15 mL) under N2 was added sodium hydride (164.54 mg (60%), 4.11 mmol) in one portion. The reaction was allowed to stir for 5 min, then a solution of 2-chloroisonicotinonitrile (300 mg, 2.17 mmol) in DMF (5 mL) was added slowly. After 5 min the reaction was allowed to warm to RT and the reaction was stirred for 2 hours. The mixture was partitioned between EtOAc (40 ml) and NH₄Cl sol. (30 ml), the organic layer was washed with brine (3×30 ml). The organic layer was dried (MgSO₄) and concentrated in vacuo to afford a crude solid, which was purified by silica gel chromatography (eluting with heptane-20% EtOAc/Heptane) to afford the desired product as a colourless liquid (385 mg, 1.56 mmol, 72.2% yield).

MS (m/z): 247 [M+H]+.

### b) {2-[(1,1,1-trifluoro-3-methoxypropan-2-yl)oxy]pyridin-4-yl}methanamine

Cobalt (II) chloride (443.0 mg, 3.41 mmol) was added to a solution of 2-[(1,1,1-trifluoro-3-methoxypropan-2-yl)oxy]pyridine-4-carbonitrile (420 mg, 1.71 mmol) in Methanol (12 mL) and THF (12 mL) under N₂ at 0° C. The reaction was stirred for 1 min, then sodium borohydride (645.4 mg, 17.06 mmol) was added portionwise over 10 min, then allowed to slowly warm to RT and left to stir o/n. EtOAc was added and filtered through a plug of celite. The filtrate was washed with water (basified with a few mls NH₃(aq)), and the aqueous layer was extracted with further EtOAc (50 ml). The combined organic layers were dried (MgSO₄) and concentrated in vacuo to afford the crude product (420 mg, 1.68 mmol, 98.4% yield) as a light brown liquid, which was taken forward without further purification.

MS (m/z): 251 [M+H]+.

### c) 1-[[2-[(2R)-1,1,1-trifluoro-3-methoxypropan-2-yl]oxypyridin-4-yl]methyl]-3-[rac-(1R,2R)-2-(trifluoromethyl)cyclopropyl]urea

1-({2-[(1,1,1-trifluoro-3-methoxypropan-2-yl)oxy]pyridin-4-yl}methyl)-3-[(1R,2R)-2-(trifluoromethyl)cyclopropyl]urea was obtained in analogy to Example 1 as a white solid (50.6% yield) using {2-[(1,1,1-trifluoro-3-methoxypropan-2-yl)oxy]pyridin-4-yl}methanamine, rac-(1R,2R)-2-(trifluoromethyl)cyclopropan1-amine hydrochloride and 4-nitrophenyl chloroformate. Purification by chiral HPLC (Cellulose-4 column, eluted with water:acetonitrile 65:35) gave the title product (22.3% yield, 100% de) as a colorless gum.

MS (m/z): 402.1 [M+H]+.

### REFERENCE

(1) Monofluoromethyl-Substituted Sulfonium Ylides: Electrophilic Monofluoromethylating Reagents with Broad Substrate Scopes. Liu, Y.; Lu, L.; Shen, Q. Angew. Chem. Int. Ed. 2017, 56, 9930-9934. [DOI: 10.1002/anie.201704175].

### BIOLOGICAL EXPERIMENTS

The potency of Kv7 potentiators was determined using the SyncroPatch 384 (Nanion) high throughput electrophysiology platform.

### Example 54 Cell Culture

CHO cells stably expressing either human Kv7.2, Kv7.4 or Kv7.5/7.3 under a constitutive CMV promoter were used for these studies. Cells were maintained in F12 Hams+1 mM L-glutamine (Hyclone) supplemented with 10% FBS (Sigma), 0.3×NEAA (Non-essential Gamino-acids) and 400 ug/ml G418 at 37° C. in 5% CO₂. Cells were cultured in T-225 flasks (Nunc) for 2-3 days to reach 85-95% confluence prior to electrophysiological recording.

### Example 55 Solutions

Solutions were of the following composition:

Earle's balanced salt solution (in mM): 135 NaCl, 5.4 KCl, 5 Glucose, 2 CaCl₂, 1 MgCl₂, 5 HEPES, pH 7.4. Seal enhancer solution (in mM): 90 NaCl, 3 KCl, 35 CaCl₂, 10 MgCl₂, 10 HEPES, pH 7.4. Extracellular recording solution (in mM): 71 NaCl, 70 NMDG, 13 KCl, 5 Glucose, 2 CaCl₂, 1 MgCl₂, 10 HEPES, pH 7.4. Intracellular recording solution (in mM): 130 KF, 20 KCl, 4 EGTA, 10 HEPES, 2 EDTA, 0.01 Escin, pH 7.2.

### Example 56 Cell Preparation

Cells were harvested for electrophysiological recording upon reaching appropriate confluence. Cells were first washed in DPBS (Hyclone, Cat #SH30028.03) and then 2 ml of Accutase (MP Biomedicals #1000449) was added at 28° C. until ˜90% of cells were suspended. F12 HAM's media+1 mM L-glutamine (Hyclone, SH30026.02) was then added to the flask to dilute the accutase. Cells were then triturated until a single cell suspension was achieved, a cell count was performed, and cells were centrifuged for 2 min. at 1,000 rpm. Media was then aspirated, and cells were resuspended in Earle's balanced salt solution to a concentration of 0.75×10⁶ cells/ml and allowed to recover for 25 min at 10° C.

### Example 57 SyncroPatch Recording

At the beginning of each assay, 20 μl of cell suspension was dispensed into each well of a multi-hole 384-well SyncroPatch chip by the onboard pipettor. Cell sealing was initiated, and seal enhancer solution was added to facilitate seal formation. Upon completion of sealing, cells were washed 3 times with extracellular recording solution and the assay voltage protocol was started. Human Kv7.2, Kv7.4 or Kv7.5/7.3 channels were evaluated using a voltage protocol in which cells were voltage-clamped at a holding potential of −60 mV. Potassium currents were continuously activated with a series of three voltage steps to −30 mV for 3 seconds, 40 mV for 1 second and −90 mV for 4 seconds with 12 seconds between successive voltage sweeps. Potassium currents were measured from the −90 mV repolarizing step. Baseline current was assessed for 3.5 minutes prior to the addition of 5.6 μM zinc pyrithione (1 μM for Kv7.4). Kv7.2, Kv7.4 or Kv7.5/7.3 current in the presence of zinc pyrithione was acquired over five minutes to allow channels to reach steady state activity prior to addition of test agents. Channel activity was monitored for three minutes preceding the addition of 30 μM ML-213 (3 minutes) to achieve maximum activation. 150 mM TEA with 10 μM XE-991 was applied for 2 minutes to measure the leak current during maximum inhibition of Kv7.2, Kv7.4 or Kv7.5/7.3 channels.

### Example 58 Data Analysis

Data were collected on the SyncroPatch platform using PatchControl software (Nanion) and processed and analyzed using DataControl Software (Nanion). Percent activation was calculated from potassium current as follows: The average current for the 5 sweeps in the presence of zinc pyrithione immediately preceding test agent addition was taken as the ‘control’ data. Likewise, the average potassium current in the presence of test agents 5 sweeps immediately preceding addition of 30 μM ML-213 was determined for ‘drug’ data. The average potassium current 5 sweeps immediately prior to addition of 150 mM TEA+10 μM XE-991 was determined for ‘max activation’ data while average currents from 5 sweeps immediately following addition of TEA was determined for ‘max inhibition’ data. Percent activation for each of the 384-wells of a sealchip was calculated as ((‘drug’−‘max inhibition’)/(‘max activation’−‘max inhibition’))*100 with Pipeline Pilot (Accelrys). Percent activation was plotted as a function of concentration and concentration-response curves were fitted with a logistic equation {Y is Bottom+(Top−Bottom)/(1+10\hat{ }((Log EC50−X)*HillSlope))} for determination of the EC₅₀ (IDBS ABASE). EC₅₀ values for human Kv7.2 are provided in Table 1.

### Example 59 Microsomal Clearance

Pooled mixed gender human liver microsomes were purchased from BioIVT at a concentration of 20 mg protein/mL and stored at −80° C. Incubations were conducted in V-bottomed 350 μL polypropylene 96-well plates. Compounds were diluted in phosphate buffer (final concentrations 0.5 μM compound, 0.5% DMSO) and mixed with diluted liver microsomes (fc 0.25 mg/mL). This mixture was aliquoted onto the assay plate (six wells per compound) and pre-warmed at 37° C. for ten minutes. Addition of NADPH (fc 0.5 mM, final incubation volume 100 μL) to each well started the time course. Reactions were stopped at 0.5, 3, 5, 10, 20 & 30 minutes by addition of 150 μL acetonitrile containing internal standard (IS; 0.125 μg/mL daidzein) and stopped samples were removed to a clean polypropylene plate. At the end of the assay the plate was refrigerated for an hour and then centrifuged at 3000 rpm for 10 minutes at 4° C. A sample of the supernatant was removed to a fresh plate and diluted 10-fold with 50:50 acetonitrile: water, then heat sealed. Analysis was by liquid chromatography coupled with tandem rmass spectrometry (LC-MS/MS) for analyte and IS levels. Ratios of the analyte and IS peak areas were generated for results calculation (IS ratio). Dextromethorphan and verapamil were included in each experiment as controls for high clearance compounds. IS ratio data were converted into natural log values which were plotted against time and a linear regression fit applied. The slope of the line was returned and converted into the elimination constant (kₑₗ) by multiplying by −1. The elimination constant was used to calculate the in vitro CLᵢₙₜ: in vitro CLᵢₙₜ (μL/min/mg protein) is kₑₗ*(1000/protein conc in mg/mL).

### Example 60

A compound of formula (I), or a solvate or a pharmaceutically acceptable salt thereof can be used in a manner known per se as the active ingredient for the production of tablets of the following composition:

Per tablet

| | | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 61

A compound of formula (I), or a solvate or a pharmaceutically acceptable salt thereof, can be used in a manner known per se as the active ingredient for the production of capsules of the following composition:

Per capsule

| | | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

### Specific Embodiments and Combinations

The following numbered clauses represent specific embodiments and combinations of features forming part of the present invention.

1. A compound of formula (I), or a solvate, or a pharmaceutically acceptable salt thereof:

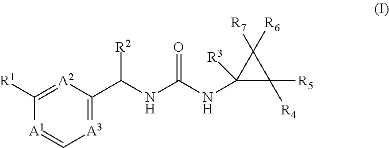

wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy;
R² is H or C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

2. A compound according to clause 1, wherein (i) only A¹; (ii) only A²; (iii) only A³; or (iii) A¹ and A² both are N.

3. A compound according to clause 2, wherein only A¹ is N.

4. A compound according to any one of clauses 1-3, wherein R¹ is selected from cyano, haloC₁₋₆alkyl, halogen, 5 membered heteroaryl, haloC₁₋₆alkoxy, 4-6 membered heterocycloalkyl, 4-6 membered cycloalkylC₀₋₆oxy, and 4-6 membered heterocycloalkylC₀₋₆alkoxy.

5. A compound according to any one of clauses 1-4, wherein R¹ is unsubstituted 5 membered heteroaryl selected from pyrazolyl, imidazolyl, oxazolyl, and thiazolyl.

6. A compound according to clause 4, wherein R¹ is (i) haloC₁₋₆alkoxy selected from CHF₂O—, CFH₂O—, CF₃O—, CHF₂CH₂O—, FCH₂CFHCH₂O—, and CF₃CH₂O— or (ii) haloC₁₋₆alkyl selected from CF₃—, CHF₂—, CH₃CF₂—, and CFH₂—.

7. A compound according to clause 6, wherein R¹ is CHF₂O—.

8. A compound according to any one of clauses 1-7, wherein R² is H.

9. A compound according to any one clauses 1-8, where R³ is H.

10. A compound according to any one of clauses 1-9, wherein both of R⁴ and R⁵ are H.

11. A compound according to any one of clauses 1-9, wherein (i) one of R⁴ and R⁵ is H and the other one is halogen; or (ii) both of R⁴ and R⁵ are halogen.

12. A compound according to clause 11, wherein both of R⁴ and R⁵ are halogen.

13. A compound according to clauses 11 or 12, wherein each halogen is F—.

14. A compound according to any one of clauses 1-13, wherein R⁶ is selected from haloC₁₋₆alkyl, phenyl and C₃₋₆ saturated monocyclic cycloalkyl, which phenyl and C₃₋₆ saturated monocyclic cycloalkyl are optionally substituted with one, two, or three substituents independently selected from halogen, haloC₁₋₆alkoxy, C₁₋₆alkyl, and C₁₋₆alkoxy.

15. A compound according to clause 14, where R⁶ is haloC₁₋₆alkyl selected from (CH₃)₂FC—, CF₃CH₂—, CH₃CF₂—, CF₃—, CH₂F—, and CHF₂—.

16. A compound according to clause 15, wherein R⁶ is CF₃—.

17. A compound according to clause 14, wherein R⁶ is unsubstituted phenyl or unsubstituted C₃₋₆ saturated monocyclic cycloalkyl.

18. A compound according to clause 17, wherein R⁶ is unsubstituted phenyl.

19. A compound according to clause 17, wherein R⁶ is unsubstituted cyclohexanyl.

20. A compound according to any one of clauses 1-19, wherein R⁷ is H.

21. A compound according to clause 1, wherein A¹ is N, A² is CH, A³ is CH, R¹ is haloC₁₋₆ alkoxy, R² is H, R³ is H, R⁴ and R⁵ are both H, R⁶ is selected from haloC₁₋₆alkyl, unsubstituted phenyl, and unsubstituted C₃₋₆ saturated monocyclic cycloalkyl, and R⁷ is H.

22. A compound according to clause 21, wherein A¹ is N, A² is CH, A³ is CH, R¹ is haloC₁₋₆ alkoxy, R² is H, R³ is H, R⁴ and R⁵ are both H, R⁶ is haloC₁₋₆alkyl, and R⁷ is H.

23. A compound according to clause 1, selected from the group consisting of:

| Example | Name | Structure |
|---|---|---|
| 1 | 1-[(4-cyanopyridin- 2-yl)methyl]-3-[(1R,2S)-2- phenylcyclopropyl]urea | 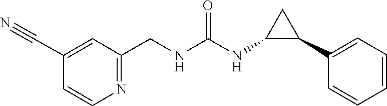 [SMILES: N#Cc1ccnc(CNC(=O)N[C@@H]2C[C@H]2c2ccccc2)c1] |
| 2 | 1-[rac-(1R,2S)-2- cyclohexylcyclopropyl]-3- [(2-pyrazol-1-ylpyridin-4- yl)methyl]urea | 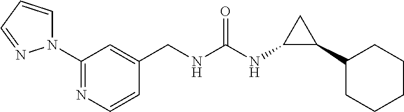 [SMILES: O=C(NCc1ccnc(-n2cccn2)c1)N[C@@H]1C[C@H]1C1CCCCC1] |
| 3 | 1-[(6-chloropyridin- 2-yl)methyl]-3-[rac- (1R,2S)-2- cyclohexylcyclopropyl]urea | 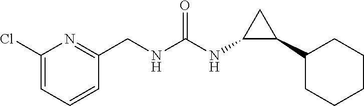 [SMILES: O=C(NCc1cccc(Cl)n1)N[C@@H]1C[C@H]1C1CCCCC1] |
| 4 | 1-[(2-propan-2- ylpyrimidin-4-yl)methyl]- 3-[rac-(1R,2S)-2- cyclohexylcyclopropyl]urea | 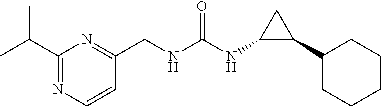 [SMILES: CC(C)c1nccc(CNC(=O)N[C@@H]2C[C@H]2C2CCCCC2)n1] |
| 5 | 1-[(2- methoxypyridin-4- yl)methyl]-3-[rac-(1R,2S)- 2-phenylcyclopropyl]urea | 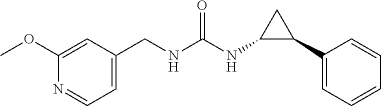 [SMILES: COc1cc(CNC(=O)N[C@@H]2C[C@H]2c2ccccc2)ccn1] |
| 6 | 1-[(1R,2S)-2- phenylcyclopropyl]-3-[(2- propan-2-yloxypyridin-4- yl)methyl]urea | 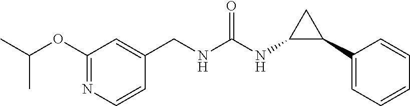 [SMILES: CC(C)Oc1cc(CNC(=O)N[C@@H]2C[C@H]2c2ccccc2)ccn1] |
| 7 | 1-[[2- (difluoromethoxy)pyridin- 4-yl]methyl]-3-[(1R,2S)-2- phenylcyclopropyl]urea | 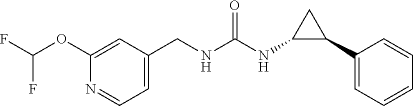 [SMILES: O=C(NCc1ccnc(OC(F)F)c1)N[C@@H]1C[C@H]1c1ccccc1] |
| 8 | 1-[(2-cyanopyridin- 4-yl)methyl]-3-[rac- (1R,2S)-2- phenylcyclopropyl]urea | 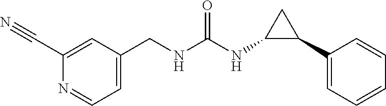 [SMILES: N#Cc1cc(CNC(=O)N[C@@H]2C[C@H]2c2ccccc2)ccn1] |
| 9 | 1-[(2- methoxypyridin-4- yl)methyl]-3-[(1R,2S)-2- phenylcyclopropyl]urea | 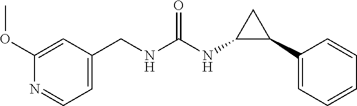 [SMILES: COc1cc(CNC(=O)N[C@@H]2C[C@H]2c2ccccc2)ccn1] |
| 10 | 1-[(2-cyanopyridin- 4-yl)methyl]-3-[(1R,2S)-2- phenylcyclopropyl]urea | 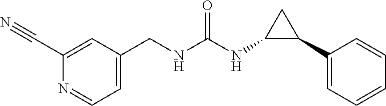 [SMILES: N#Cc1cc(CNC(=O)N[C@@H]2C[C@H]2c2ccccc2)ccn1] |
| 11 | 1-(2- cyclohexylcyclopropyl)-3- [(2-imidazol-1-ylpyridin-4- yl)methyl]urea | 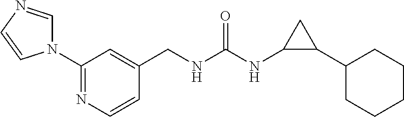 [SMILES: O=C(NCc1ccnc(-n2ccnc2)c1)NC1CC1C1CCCCC1] |
| 12 | 1-[(2-pyrazol-1- ylpyridin-4-yl)methyl]-3- [rac-(1R,2S)-2- phenylcyclopropyl]urea | 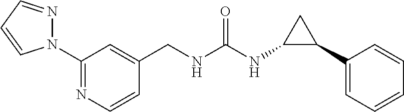 [SMILES: O=C(NCc1ccnc(-n2cccn2)c1)N[C@@H]1C[C@H]1c1ccccc1] |
| 13 | 1-[(1R,2S)-2- phenylcyclopropyl]-3-[(2- pyrazol-1-ylpyridin-4- yl)methyl]urea | 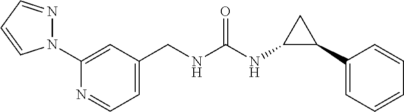 [SMILES: O=C(NCc1ccnc(-n2cccn2)c1)N[C@@H]1C[C@H]1c1ccccc1] |
| 14 | 1-[(1R,2S)-2- phenylcyclopropyl]-3-[2- (2,2,2- trifluoroethoxy)pyridin-4- yl]methyl]urea | 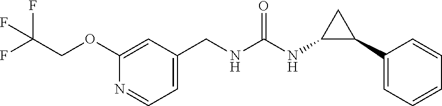 [SMILES: O=C(NCc1ccnc(OCC(F)(F)F)c1)N[C@@H]1C[C@H]1c1ccccc1] |
| 15 | 1-[(1S,2R)-2- cyclohexylcyclopropyl]-3- [(2-imidazol-1-ylpyridin-4- yl)methyl]urea | 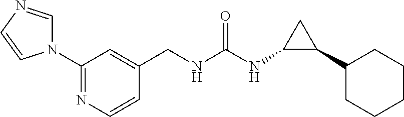 [SMILES: O=C(NCc1ccnc(-n2ccnc2)c1)N[C@@H]1C[C@H]1C1CCCCC1] |
| 16 | 1-[[2-(oxetan-3- yloxy)pyridin-4- yl]methyl]-3-[(1R,2S)-2- phenylcyclopropyl]urea | 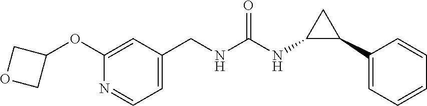 [SMILES: O=C(NCc1ccnc(OC2COC2)c1)N[C@@H]1C[C@H]1c1ccccc1] |
| 17 | 1-[(2-chloropyridin- 4-yl)methyl]-3-[rac- (1R,2S)-2- cyclohexylcyclopropyl] urea | 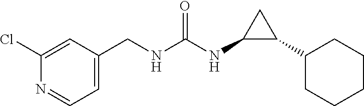 [SMILES: O=C(NCc1ccnc(Cl)c1)N[C@H]1C[C@@H]1C1CCCCC1] |
| 18 | 1-[(6-cyanopyridin- 2-yl)methyl]-3-[(1R,2S)-2- phenylcyclopropyl]urea | 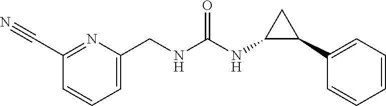 [SMILES: N#Cc1cccc(CNC(=O)N[C@@H]2C[C@H]2c2ccccc2)n1] |
| 19 | 1-[(2-chloropyridin- 4-yl)methyl]-3-[(1R,2S)-2- phenylcyclopropyl]urea | 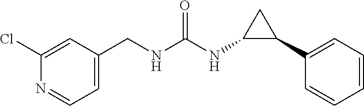 [SMILES: O=C(NCc1ccnc(Cl)c1)N[C@@H]1C[C@H]1c1ccccc1] |
| 20 | 1-[[2-(oxan-4- yloxy)pyridin-4- yl]methyl]-3-[(1R,2S)-2- phenylcyclopropyl]urea | 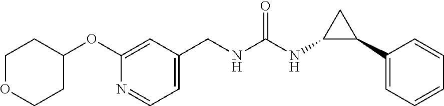 [SMILES: O=C(NCc1ccnc(OC2CCOCC2)c1)N[C@@H]1C[C@H]1c1ccccc1] |
| 21 | 1-[[2- (difluoromethyl)pyridin-4- yl]methyl]-3-[(1R,2S)-2- phenylcyclopropyl]urea | 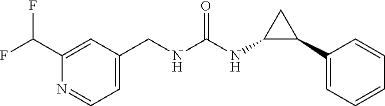 [SMILES: O=C(NCc1ccnc(C(F)F)c1)N[C@@H]1C[C@H]1c1ccccc1] |
| 22 | 1-[(1R,2S)-2- phenylcyclopropyl]-3-[2- (trifluoromethyl)pyridin-4- yl]methyl]urea | 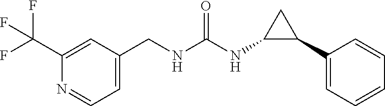 [SMILES: O=C(NCc1ccnc(C(F)(F)F)c1)N[C@@H]1C[C@H]1c1ccccc1] |
| 23 | 1-[[2-(1,3-oxazol-5- yl)pyridin-4-yl]methyl]-3- [rac-(1R,2S)-2- cyclohexylcyclopropyl] urea | 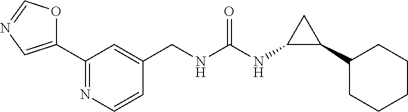 [SMILES: O=C(NCc1ccnc(-c2cnco2)c1)N[C@@H]1C[C@H]1C1CCCCC1] |
| 24 | 1-[rac-(1R,2S)-2- cyclohexylcyclopropyl]-3- [[2-[4- (trifluoromethyl)imidazol- 1-yl]pyridin-4- yl]methyl]urea | 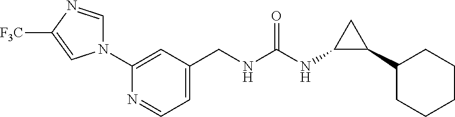 [SMILES: O=C(NCc1ccnc(-n2cnc(C(F)(F)F)c2)c1)N[C@@H]1C[C@H]1C1CCCCC1] |
| 25 | 1-[(2- cyclobutyloxypyridin-4- yl)methyl]-3-[(1R,2S)-2- phenylcyclopropyl]urea | 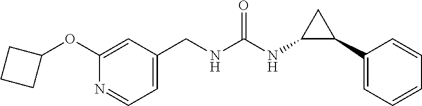 [SMILES: O=C(NCc1ccnc(OC2CCC2)c1)N[C@@H]1C[C@H]1c1ccccc1] |
| 26 | 1-[[2-(1,1- difluoroethyl)pyridin-4- yl]methyl]-3-[(1R,2S)-2- phenylcyclopropyl]urea | 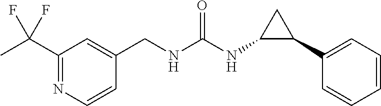 [SMILES: CC(F)(F)c1cc(CNC(=O)N[C@@H]2C[C@H]2c2ccccc2)ccn1] |
| 27 | 1-[(2-imidazol-1- ylpyridin-4-yl)methyl]-3- [(1R,2S)-2- phenylcyclopropyl]urea | 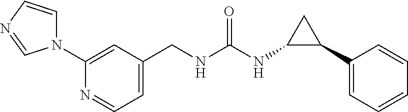 [SMILES: O=C(NCc1ccnc(-n2ccnc2)c1)N[C@@H]1C[C@H]1c1ccccc1] |
| 28 | 1-[(1R,2S)-2- phenylcyclopropyl]-3-[2- (trifluoromethoxy)pyridin- 4-yl]methyl]urea | 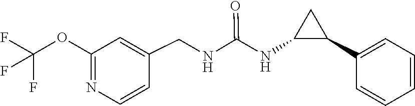 [SMILES: O=C(NCc1ccnc(OC(F)(F)F)c1)N[C@@H]1C[C@H]1c1ccccc1] |
| 29 | 1-[(1S)-1-(2- methoxypyridin-4- yl)ethyl]-3-[(1R,2S)-2- phenylcyclopropyl]urea | 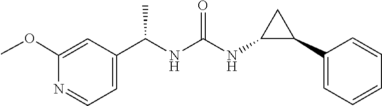 [SMILES: COc1cc([C@H](C)NC(=O)N[C@@H]2C[C@H]2c2ccccc2)ccn1] |
| 30 | 1-((S)-1-(2- (difluoromethoxy)pyridin- 4-yl)ethyl)-3-((1R,2S)-2- phenylcyclopropyl)urea | 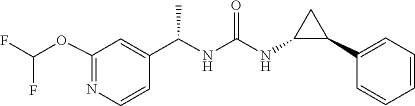 [SMILES: C[C@H](NC(=O)N[C@@H]1C[C@H]1c1ccccc1)c1ccnc(OC(F)F)c1] |
| 31 | 1-[(1R,2S)-2- phenylcyclopropyl]-3-[[2- [4- (trifluoromethyl)imidazol- 1-yl]pyridin-4- yl]methyl]urea | 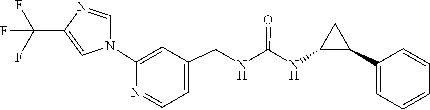 [SMILES: O=C(NCc1ccnc(-n2cnc(C(F)(F)F)c2)c1)N[C@@H]1C[C@H]1c1ccccc1] |
| 32 | 1-[[2-(oxetan-3- yl)pyridin-4-yl]methyl]-3- [(1R,2S)-2- phenylcyclopropyl]urea | 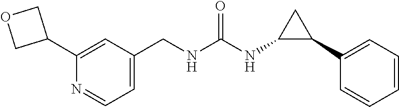 [SMILES: O=C(NCc1ccnc(C2COC2)c1)N[C@@H]1C[C@H]1c1ccccc1] |
| 33 | 1-[(1R,2S)-2- phenylcyclopropyl]-3- [(1S)-1-[2-[4- (trifluoromethyl)imidazol- 1-yl]pyridin-4- yl]ethyl]urea | 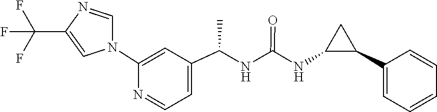 [SMILES: C[C@H](NC(=O)N[C@@H]1C[C@H]1c1ccccc1)c1ccnc(-n2cnc(C(F)(F)F)c2)c1] |
| 34 | 1-[(2- phenoxypyridin-4- yl)methyl]-3-[(1R,2S)-2- phenylcyclopropyl]urea | 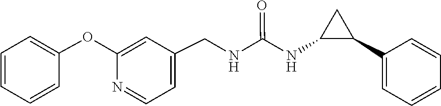 [SMILES: O=C(NCc1ccnc(Oc2ccccc2)c1)N[C@@H]1C[C@H]1c1ccccc1] |
| 35 | 1-[[2-(2,2- difluoroethoxy)pyridin-4- yl]methyl]-3-[(1R,2S)-2- phenylcyclopropyl]urea | 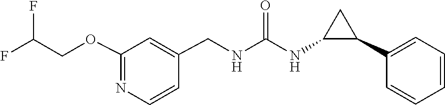 [SMILES: O=C(NCc1ccnc(OCC(F)F)c1)N[C@@H]1C[C@H]1c1ccccc1] |
| 36 | 1-[[2- (difluoromethoxy)pyridin- 4-yl]methyl]-3-[rac- (1R,2S)-1-methyl-2- phenylcyclopropyl]urea | 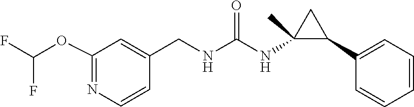 [SMILES: C[C@@]1(NC(=O)NCc2ccnc(OC(F)F)c2)C[C@H]1c1ccccc1] |
| 37 | 1-[[2- (fluoromethoxy)pyridin-4- yl]methyl]-3-[(1R,2S)-2- phenylcyclopropyl]urea | 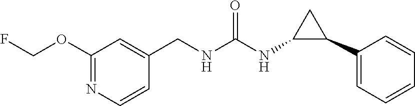 [SMILES: O=C(NCc1ccnc(OCF)c1)N[C@@H]1C[C@H]1c1ccccc1] |
| 38 | 1-((R)-1-(2- (difluoromethoxy)pyridin- 4-yl)ethyl)-3-((1R,2S)-2- phenylcyclopropyl)urea | 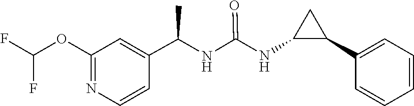 [SMILES: C[C@@H](NC(=O)N[C@@H]1C[C@H]1c1ccccc1)c1ccnc(OC(F)F)c1] |
| 39 | 1-[(1R,2S)-2- phenylcyclopropyl]-3-[2- [3- (trifluoromethyl)pyrazol-1- yl]pyridin-4- yl]methyl]urea | 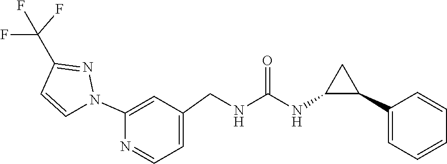 [SMILES: O=C(NCc1ccnc(-n2ccc(C(F)(F)F)n2)c1)N[C@@H]1C[C@H]1c1ccccc1] |
| 40 | 1-[rac-(1R,2S)-2- cyclohexylcyclopropyl]-3- [[2-(1,2-thiazol-4- yl)pyridin-4- yl]methyl]urea | 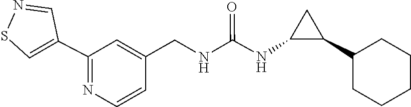 [SMILES: O=C(NCc1ccnc(-c2cnsc2)c1)N[C@@H]1C[C@H]1C1CCCCC1] |
| 41 | 1-[[2- (difluoromethoxy)pyridin- 4-yl]methyl]-3-[(1R,2R)-2- (trifluoromethyl)cyclopropyl] urea | 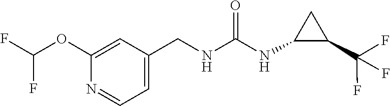 [SMILES: O=C(NCc1ccnc(OC(F)F)c1)N[C@@H]1C[C@H]1C(F)(F)F] |
| 42 | 1-[[2- (difluoromethoxy)pyridin- 4-yl]methyl]-3-[rac- (1R,3R)-2,2-difluoro-3- methylcyclopropyl]urea | 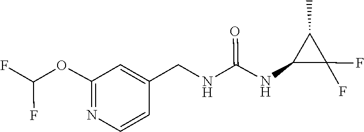 [SMILES: C[C@H]1[C@H](NC(=O)NCc2ccnc(OC(F)F)c2)C1(F)F] |
| 43 | 1-[[2- (difluoromethoxy)pyridin- 4-yl]methyl]-3-[rac- (1R,2R)-2-(oxolan-2- yl)cyclopropyl]urea | 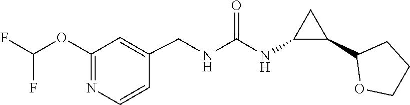 [SMILES: O=C(NCc1ccnc(OC(F)F)c1)N[C@@H]1C[C@H]1C1CCCO1] |
| 44 | 1-[[2- (difluoromethoxy)pyridin- 4-yl]methyl]-3-[rac- (1R,2R)-2-(1,1- difluoroethyl)cyclopropyl] urea | 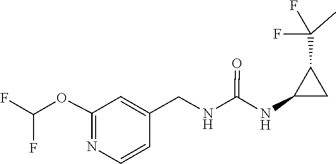 [SMILES: CC(F)(F)[C@@H]1C[C@H]1NC(=O)NCc1ccnc(OC(F)F)c1] |
| 45 | 1-(2- cyclohexylcyclopropyl)-3- [[2-(oxan-4-yloxy)pyridin- 4-yl]methyl]urea | 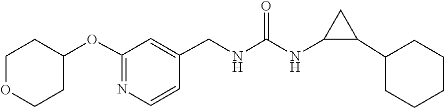 [SMILES: O=C(NCc1ccnc(OC2CCOCC2)c1)NC1CC1C1CCCCC1] |
| 46 | 1-[[2- (difluoromethoxy)pyridin- 4-yl]methyl]-3-[rac- (1R,2R)-2- methylcyclopropyl]urea | 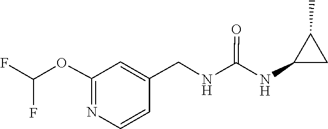 [SMILES: C[C@@H]1C[C@H]1NC(=O)NCc1ccnc(OC(F)F)c1] |
| 47 | 1-[[2- (difluoromethoxy)pyridin- 4-yl]methyl]-3-[rac- (1R,2R)-2-methyl-2- (trifluoromethyl)cyclopropyl] urea | 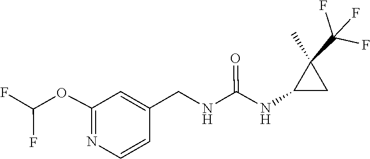 [SMILES: C[C@]1(C(F)(F)F)C[C@@H]1NC(=O)NCc1ccnc(OC(F)F)c1] |
| 48 | 1-[[2-(2,3- difluoropropoxy)pyridin-4- yl]methyl]-3-[(1R,2S)-2- phenylcyclopropyl]urea | 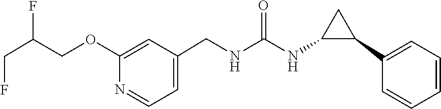 [SMILES: O=C(NCc1ccnc(OCC(F)CF)c1)N[C@@H]1C[C@H]1c1ccccc1] |
| 49 | 1-[[2- (difluoromethoxy)pyrimidin- 4-yl]methyl]-3-[(1R,2R)- 2- (trifluoromethyl)cyclopropyl] urea | 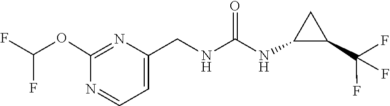 [SMILES: O=C(NCc1ccnc(OC(F)F)n1)N[C@@H]1C[C@H]1C(F)(F)F] |

24. A compound of formula (I), or a solvate or a pharmaceutically acceptable salt thereof, according to any one of clauses 1-23, wherein such compounds of formula (I) show Kv7.2 EC50 values and/or Kv7.3_7.5/Kv7.2 selectivity ratios in the range of: (i) EC50<3 μM, Select.>10×, or (ii) EC50<1 μM, Select.>30×.

25. A pharmaceutical composition comprising a compound of formula (I), or a solvate or a pharmaceutically acceptable salt thereof according to any one of clauses 1-24, for use in the therapeutic and/or prophylactic treatment of a disorder, disease, or disability associated with Kv7.2 in a subject in need thereof.

26. A compound of formula (I), or a solvate or a pharmaceutically acceptable salt thereof according to any one of clauses 1-24; or a pharmaceutical composition according to clause 25; for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

27. The compound or pharmaceutical composition for use according to clause 26, wherein the disorder, disease, or disability is a behavioral disorder which is Attention Deficit Hyperactivity Disorder (ADHD).

28. The compound or pharmaceutical composition for use according to clause 26, wherein the disorder, disease, or disability is a mood disorder which is depression.

29. The compound or pharmaceutical composition for use according to clause 26, wherein the disorder, disease, or disability is a neurodevelopment disorder selected from autism spectrum disorder (ASD) and syndromic developmental disorders.

30. The compound or pharmaceutical composition for use according to clause 26, wherein the disorder, disease, or disability is a syndromic developmental disorder selected from Dup15q syndrome (Dup15q), Fragile X syndrome (FXS) and Angelman syndrome.

31. The compound or pharmaceutical composition for use according to clause 26, wherein the disorder, disease, or disability is an epilepsy selected from broad pediatric epilepsy, West syndrome, Ohtahara syndrome, and epileptic encephalopathy.

32. The compound or pharmaceutical composition for use according to clause 26, wherein disorder, disease, or disability is a neurodegenerative disease selected from Alzheimer's disease, and motor neuron diseases.

33. The compound or pharmaceutical composition for use according to any one of clauses 26-32, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

34. A compound according to any one of clauses 1-24, or a pharmaceutical composition according to clause 25 for use in therapy.

35. A compound according to any one of clauses 1-24, or a pharmaceutical composition according to clause 25, or a pharmaceutical composition for use according to any one of clauses 26-32, for the manufacture of a medicament for use in the therapeutic and/or prophylactic treatment of a disorder, disease, or disability associated with Kv7.2.

36. A method for the therapeutic and/or prophylactic treatment of a disorder, disease, or disability associated with Kv7.2, which method comprises administering a therapeutically effective amount of a compound according to any one of clauses 1-24, or a pharmaceutical composition according to clause 25, or a pharmaceutical composition for use according to any one of clauses 26-32.

37. A kit for use in the therapeutic and/or prophylactic treatment of a disorder, disease, or disability associated with Kv7.2 comprising:
a) a compound according to any one of clauses 1-24, or a pharmaceutical composition according to clause 25, or a pharmaceutical composition for use according to any one of clauses 26-32; and
b) instructions for use.

38. The invention as hereinbefore described.

1. A compound of formula (I′), or a solvate, or a pharmaceutically acceptable salt thereof: 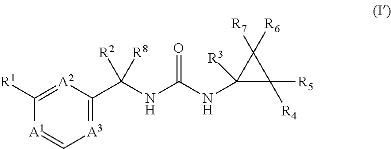
wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy; and wherein haloC₁₋₆alkoxy is optionally substituted with C₁₋₆alkoxy;
R² is selected from H, D, and C₁₋₆alkyl;
R³ is H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen; and
R⁸ is selected from H, D, and C₁₋₆alkyl.

2. A compound according to claim 1 wherein the compound is of formula (I), or a solvate or a pharmaceutically acceptable salt thereof: 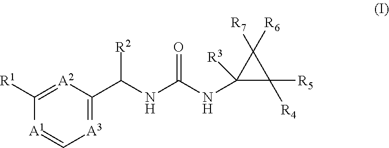
wherein
A¹ is N or CH;
A² is N or CH;
A³ is N or CH; with the proviso that at least one of A¹, A², or A³ is N;
R¹ is selected from cyano, haloC₁₋₆alkyl, halogen, 5 membered heteroaryl, C₁₋₆alkyl, C₁₋₆alkoxy, haloC₁₋₆alkoxy, 3-6 membered cycloalkylC₀₋₆alkoxy, 4-6 membered heterocycloalkyl, 4-6 membered heterocycloalkylC₀₋₆alkoxy, and phenoxy, wherein heteroaryl, heterocycloalkyl, heterocycloalkyloxy, cycloalkyloxy or phenoxy are optionally substituted with one, two, or three substituents independently selected from halogen, haloC₁₋₆alkyl, and haloC₁₋₆alkoxy;
R² is H, or C₁₋₆alkyl;
R³ is selected from H or C₁₋₆alkyl;
R⁴ is H or halogen;
R⁵ is H or halogen;
R⁶ is selected from phenyl, saturated monocyclic 3-6 membered cycloalkyl, C₁₋₆alkyl, 4-6 membered heterocycloalkyl, and haloC₁₋₆alkyl, wherein phenyl, cycloalkyl, or heterocycloalkyl are optionally substituted with one, two, or three substituents independently selected from halogen, haloC₁₋₆alkyl, and haloC₁₋₆alkoxy; and
R⁷ is selected from H, C₁₋₆alkyl, haloC₁₋₆alkyl, and halogen.

3. A compound according to claim 1 or 2, wherein (i) only A¹; (ii) only A²; (iii) only A³; (iv) A¹ and A² both are N, or (v) A¹ and A³ both are N.

4. A compound according to claim 3, wherein (i) only A¹ is N or (v) A¹ and A³ both are N.

5. A compound according to any one of claims 1-4, wherein R¹ is selected from cyano, haloC₁₋₆alkyl, halogen, 5 membered heteroaryl, haloC₁₋₆alkoxy, 4-6 membered heterocycloalkyl, 4-6 membered cycloalkylC₀₋₆oxy, and 4-6 membered heterocycloalkylC₀₋₆alkoxy.

6. A compound according to any one of claims 1-5, wherein R¹ is unsubstituted 5 membered heteroaryl selected from pyrazolyl, imidazolyl, oxazolyl, and thiazolyl.

7. A compound according to claim 5, wherein R¹ is (i) haloC₁₋₆alkoxy selected from CHF₂O—, CFH₂O—, CF₃O—, CHF₂CH₂O—, FCH₂CFHCH₂O—, and CF₃CH₂O—, which is optionally substituted with C₁₋₆alkoxy, o (ii) haloC₁₋₆alkyl selected from CF₃—, CHF₂—, CH₃CF₂—, and CFH₂—.

8. A compound according to claim 7, wherein R¹ is CHF₂O— or CF₃CH₂O—.

9. A compound according to any one of claims 1-8, wherein R² is H or D.

10. A compound according to any one claims 1-9, where R³ is H.

11. A compound according to any one of claims 1-10, wherein both of R⁴ and R⁵ are H.

12. A compound according to any one of claims 1-10, wherein (i) one of R⁴ and R⁵ is H and the other one is halogen; or (ii) both of R⁴ and R⁵ are halogen.

13. A compound according to claim 12, wherein both of R⁴ and R⁵ are halogen.

14. A compound according to claims 12 or 13, wherein each halogen is F—.

15. A compound according to any one of claims 1-14, wherein R⁶ is selected from haloC₁₋₆alkyl, phenyl and C₃₋₆ saturated monocyclic cycloalkyl, which phenyl and C₃₋₆ saturated monocyclic cycloalkyl are optionally substituted with one, two, or three substituents independently selected from halogen, haloC₁₋₆alkoxy, C₁₋₆alkyl, and C₁₋₆alkoxy.

16. A compound according to claim 15, where R⁶ is haloC₁₋₆alkyl selected from (CH₃)₂FC—, CF₃CH₂—, CH₃CF₂—, CF₃—, CH₂F—, and CHF₂—.

17. A compound according to claim 16, wherein R⁶ is CF₃—.

18. A compound according to claim 15, wherein R⁶ is unsubstituted phenyl or unsubstituted C₃₋₆ saturated monocyclic cycloalkyl.

19. A compound according to claim 18, wherein R⁶ is unsubstituted phenyl.

20. A compound according to claim 18, wherein R⁶ is unsubstituted cyclohexanyl.

21. A compound according to any one of claims 1-20, wherein R⁷ is H.

22. A compound according to any one of claims 1-21, wherein R¹ is D.

23. A compound according to any one of claims 1-21, wherein A¹ is N, A² is CH, A³ is CH, R¹ is haloC₁₋₆alkoxy, R² is H, R³ is H, R⁴ and R⁵ are both H, R⁶ is selected from haloC₁₋₆alkyl, unsubstituted phenyl, and unsubstituted C₃₋₆ saturated monocyclic cycloalkyl, R⁷ is H, and R¹ is H.

24. A compound according to any one of claims 1-21, wherein A¹ is N, A² is CH, A³ is CH, R¹ is haloC₁₋₆alkoxy, R² is D, R³ is H, R⁴ and R⁵ are both H, R⁶ is selected from haloC₁₋₆alkyl, unsubstituted phenyl, and unsubstituted C₃₋₆ saturated monocyclic cycloalkyl, R⁷ is H, and R⁸ is D.

25. A compound according to claim 23, wherein A¹ is N, A² is CH, A³ is CH, R¹ is haloC₁₋₆alkoxy, R² is H, R³ is H, R⁴ and R⁵ are both H, R⁶ is haloC₁₋₆alkyl, R⁷ is H, and R⁸ is H.

26. A compound according to claim 24, wherein A¹ is N, A² is CH, A³ is CH, R¹ is haloC₁₋₆ alkoxy, R² is D, R³ is H, R⁴ and R⁵ are both H, R⁶ is haloC₁₋₆alkyl, R⁷ is H, and R⁸ is D.

27. A compound according to any one of claims 1-26, selected from the group consisting of:

| Example | Name | Structure |
|---|---|---|
| 1 | 1-[(4-cyanopyridin- 2-yl)methyl]-3-[(1R,2S)-2- phenylcyclopropyl]urea | 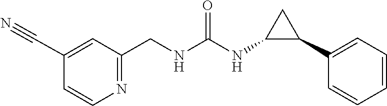 [SMILES: N#Cc1ccnc(CNC(=O)N[C@@H]2C[C@H]2c2ccccc2)c1] |
| 2 | 1-[rac-(1R,2S)-2- cyclohexylcyclopropyl]-3- [(2-pyrazol-1-ylpyridin-4- yl)methyl]urea | 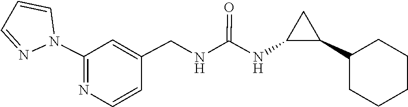 [SMILES: O=C(NCc1ccnc(-n2cccn2)c1)N[C@@H]1C[C@H]1C1CCCCC1] |
| 3 | 1-[(6-chloropyridin- 2-yl)methyl]-3-[rac- (1R,2S)-2- cyclohexylcyclopropyl] urea | 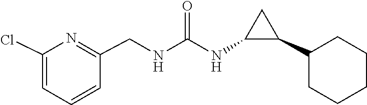 [SMILES: O=C(NCc1cccc(Cl)n1)N[C@@H]1C[C@H]1C1CCCCC1] |
| 4 | 1-[(2-propan-2- ylpyrimidin-4-yl)methyl]- 3-[rac-(1R,2S)-2- cyclohexylcyclopropyl] urea | 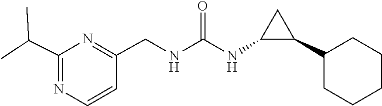 [SMILES: CC(C)c1nccc(CNC(=O)N[C@@H]2C[C@H]2C2CCCCC2)n1] |
| 5 | 1-[(2- methoxypyridin-4- yl)methyl]-3-[rac-(1R,2S)- 2-phenylcyclopropyl]urea | 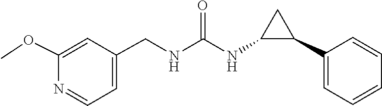 [SMILES: COc1cc(CNC(=O)N[C@@H]2C[C@H]2c2ccccc2)ccn1] |
| 6 | 1-[(1R,2S)-2- phenylcyclopropyl]-3-[(2- propan-2-yloxypyridin-4- yl)methyl]urea | 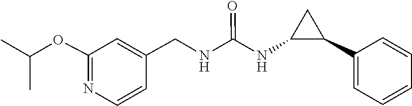 [SMILES: CC(C)Oc1cc(CNC(=O)N[C@@H]2C[C@H]2c2ccccc2)ccn1] |
| 7 | 1-[[2- (difluoromethoxy)pyridin- 4-yl]methyl]-3-[(1R,2S)-2- phenylcyclopropyl]urea | 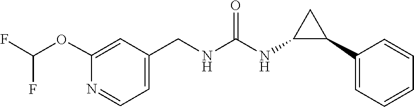 [SMILES: O=C(NCc1ccnc(OC(F)F)c1)N[C@@H]1C[C@H]1c1ccccc1] |
| 8 | 1-[(2-cyanopyridin- 4-yl)methyl]-3-[rac- (1R,2S)-2- phenylcyclopropyl]urea | 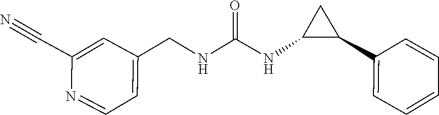 [SMILES: N#Cc1cc(CNC(=O)N[C@@H]2C[C@H]2c2ccccc2)ccn1] |
| 9 | 1-[(2- methoxypyridin-4- yl)methyl]-3-[(1R,2S)-2- phenylcyclopropyl]urea | 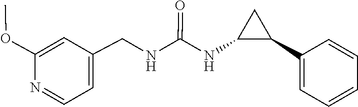 [SMILES: COc1cc(CNC(=O)N[C@@H]2C[C@H]2c2ccccc2)ccn1] |
| 10 | 1-[(2-cyanopyridin- 4-yl)methyl]-3-[(1R,2S)-2- phenylcyclopropyl]urea | 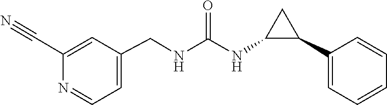 [SMILES: N#Cc1cc(CNC(=O)N[C@@H]2C[C@H]2c2ccccc2)ccn1] |
| 11 | 1-(2- cyclohexylcyclopropyl)-3- [(2-imidazol-1-ylpyridin-4- yl)methyl]urea | 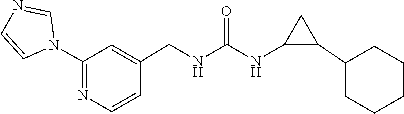 [SMILES: O=C(NCc1ccnc(-n2ccnc2)c1)NC1CC1C1CCCCC1] |
| 12 | 1-[(2-pyrazol-1- ylpyridin-4-yl)methyl]-3- [rac-(1R,2S)-2- phenylcyclopropyl]urea | 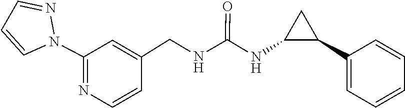 [SMILES: O=C(NCc1ccnc(-n2cccn2)c1)N[C@@H]1C[C@H]1c1ccccc1] |
| 13 | 1-[(1R,2S)-2- phenylcyclopropyl]-3-[(2- pyrazol-1-ylpyridin-4- yl)methyl]urea | 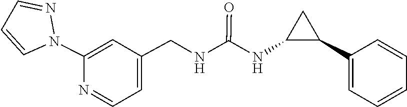 [SMILES: O=C(NCc1ccnc(-n2cccn2)c1)N[C@@H]1C[C@H]1c1ccccc1] |
| 14 | 1-[(1R,2S)-2- phenylcyclopropyl]-3-[[2- (2,2,2- trifluoroethoxy)pyridin-4- yl]methyl]urea | 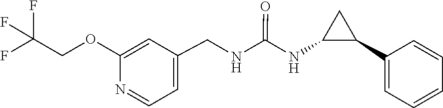 [SMILES: O=C(NCc1ccnc(OCC(F)(F)F)c1)N[C@@H]1C[C@H]1c1ccccc1] |
| 15 | 1-[(1S,2R)-2- cyclohexylcyclopropyl]-3- [(2-imidazol-1-ylpyridin-4- yl)methyl]urea | 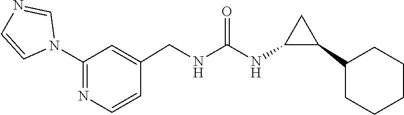 [SMILES: O=C(NCc1ccnc(-n2ccnc2)c1)N[C@@H]1C[C@H]1C1CCCCC1] |
| 16 | 1-[2-(oxetan-3- yloxy)pyridin-4- yl]methyl]-3-[(1R,2S)-2- phenylcyclopropyl]urea | 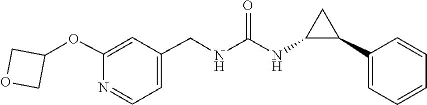 [SMILES: O=C(NCc1ccnc(OC2COC2)c1)N[C@@H]1C[C@H]1c1ccccc1] |
| 17 | 1-[(2-chloropyridin- 4-yl)methyl]-3-[rac- (1R,2S)-2- cyclohexylcyclopropyl]urea | 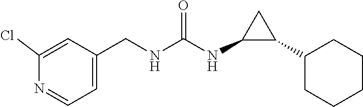 [SMILES: O=C(NCc1ccnc(Cl)c1)N[C@H]1C[C@@H]1C1CCCCC1] |
| 18 | 1-[(6-cyanopyridin- 2-yl)methyl]-3-[(1R,2S)-2- phenylcyclopropyl]urea | 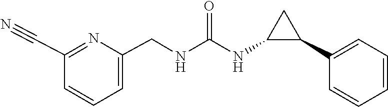 [SMILES: N#Cc1cccc(CNC(=O)N[C@@H]2C[C@H]2c2ccccc2)n1] |
| 19 | 1-[(2-chloropyridin- 4-yl)methyl]-3-[(1R,2S)-2- phenylcyclopropyl]urea | 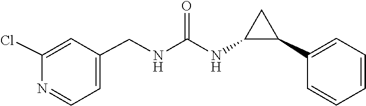 [SMILES: O=C(NCc1ccnc(Cl)c1)N[C@@H]1C[C@H]1c1ccccc1] |
| 20 | 1-[[2-(oxan-4- yloxy)pyridin-4- yl]methyl]-3-[(1R,2S)-2- phenylcyclopropyl]urea | 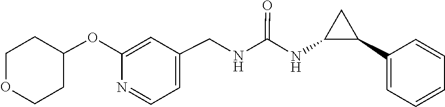 [SMILES: O=C(NCc1ccnc(OC2CCOCC2)c1)N[C@@H]1C[C@H]1c1ccccc1] |
| 21 | 1-[[2- (difluoromethyl)pyridin-4- yl]methyl]-3-[(1R,2S)-2- phenylcyclopropyl]urea | 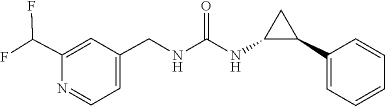 [SMILES: O=C(NCc1ccnc(C(F)F)c1)N[C@@H]1C[C@H]1c1ccccc1] |
| 22 | 1-[(1R,2S)-2- phenylcyclopropyl]-3-[[2- (trifluoromethyl)pyridin-4- yl]methyl]urea | 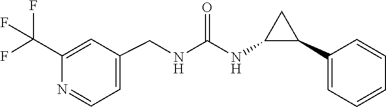 [SMILES: O=C(NCc1ccnc(C(F)(F)F)c1)N[C@@H]1C[C@H]1c1ccccc1] |
| 23 | 1-[2-(1,3-oxazol-5- yl)pyridin-4-yl]methyl]-3- [rac-(1R,2S)-2- cyclohexylcyclopropyl]urea | 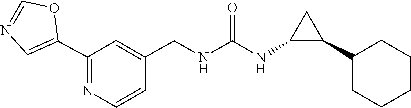 [SMILES: O=C(NCc1ccnc(-c2cnco2)c1)N[C@@H]1C[C@H]1C1CCCCC1] |
| 24 | 1-[rac-(1R,2S)-2- cyclohexylcyclopropyl]-3- [2-[4- (trifluoromethyl)imidazol- 1-yl]pyridin-4- yl]methyl]urea | 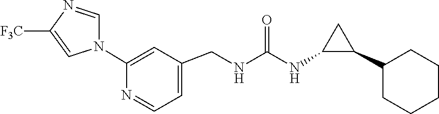 [SMILES: O=C(NCc1ccnc(-n2cnc(C(F)(F)F)c2)c1)N[C@@H]1C[C@H]1C1CCCCC1] |
| 25 | 1-[(2- cyclobutyloxypyridin-4- yl)methyl]-3-[(1R,2S)-2- phenylcyclopropyl]urea | 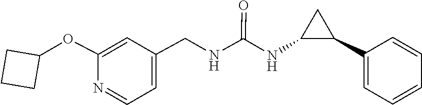 [SMILES: O=C(NCc1ccnc(OC2CCC2)c1)N[C@@H]1C[C@H]1c1ccccc1] |
| 26 | 1-[[2-(1,1- difluoroethyl)pyridin-4- yl]methyl]-3-[(1R,2S)-2- phenylcyclopropyl]urea | 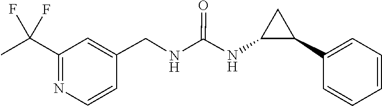 [SMILES: CC(F)(F)c1cc(CNC(=O)N[C@@H]2C[C@H]2c2ccccc2)ccn1] |
| 27 | 1-[(2-imidazol-1- ylpyridin-4-yl)methyl]-3- [(1R,2S)-2- phenylcyclopropyl]urea | 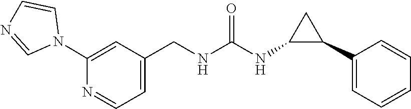 [SMILES: O=C(NCc1ccnc(-n2ccnc2)c1)N[C@@H]1C[C@H]1c1ccccc1] |
| 28 | 1-[(1R,2S)-2- phenylcyclopropyl]-3-[[2- (trifluoromethoxy)pyridin- 4-yl]methyl]urea | 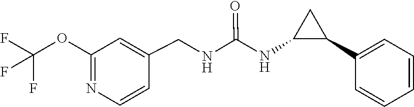 [SMILES: O=C(NCc1ccnc(OC(F)(F)F)c1)N[C@@H]1C[C@H]1c1ccccc1] |
| 29 | 1-[(1S)-1-(2- methoxypyridin-4- yl)ethyl]-3-[(1R,2S)-2- phenylcyclopropyl]urea | 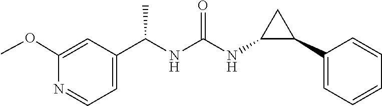 [SMILES: COc1cc([C@H](C)NC(=O)N[C@@H]2C[C@H]2c2ccccc2)ccn1] |
| 30 | 1-((S)-1-(2- (difluoromethoxy)pyridin- 4-yl)ethyl)-3-((1R,2S)-2- phenylcyclopropyl)urea | 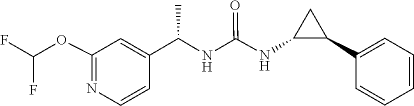 [SMILES: C[C@H](NC(=O)N[C@@H]1C[C@H]1c1ccccc1)c1ccnc(OC(F)F)c1] |
| 31 | 1-[(1R,2S)-2- phenylcyclopropyl]-3-[2- [4- (trifluoromethyl)imidazol- 1-yl]pyridin-4- yl]methyl]urea | 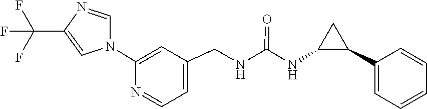 [SMILES: O=C(NCc1ccnc(-n2cnc(C(F)(F)F)c2)c1)N[C@@H]1C[C@H]1c1ccccc1] |
| 32 | 1-[[2-(oxetan-3- yl)pyridin-4-yl]methyl]-3- [(1R,2S)-2- phenylcyclopropyl]urea | 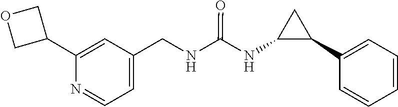 [SMILES: O=C(NCc1ccnc(C2COC2)c1)N[C@@H]1C[C@H]1c1ccccc1] |
| 33 | 1-[(1R,2S)-2- phenylcyclopropyl]-3- [(1S)-1-[2-[4- (trifluoromethyl)imidazol- 1-yl]pyridin-4- yl]ethyl]urea | 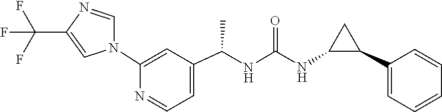 [SMILES: C[C@H](NC(=O)N[C@@H]1C[C@H]1c1ccccc1)c1ccnc(-n2cnc(C(F)(F)F)c2)c1] |
| 34 | 1-[(2- phenoxypyridin-4- yl)methyl]-3-[(1R,2S)-2- phenylcyclopropyl]urea | 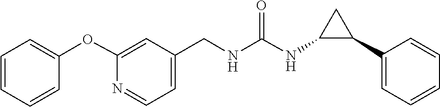 [SMILES: O=C(NCc1ccnc(Oc2ccccc2)c1)N[C@@H]1C[C@H]1c1ccccc1] |
| 35 | 1-[[2-(2,2- difluoroethoxy)pyridin-4- yl]methyl]-3-[(1R,2S)-2- phenylcyclopropyl]urea | 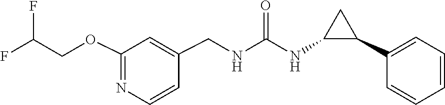 [SMILES: O=C(NCc1ccnc(OCC(F)F)c1)N[C@@H]1C[C@H]1c1ccccc1] |
| 36 | 1-[[2- (difluoromethoxy)pyridin- 4-yl]methyl]-3-[rac- (1R,2S)-1-methyl-2- phenylcyclopropyl]urea | 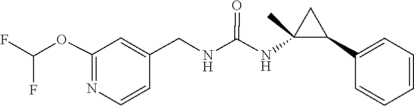 [SMILES: C[C@@]1(NC(=O)NCc2ccnc(OC(F)F)c2)C[C@H]1c1ccccc1] |
| 37 | 1-[[2- (fluoromethoxy)pyridin-4- yl]methyl]-3-[(1R,2S)-2- phenylcyclopropyl]urea | 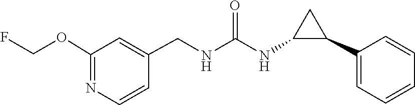 [SMILES: O=C(NCc1ccnc(OCF)c1)N[C@@H]1C[C@H]1c1ccccc1] |
| 38 | 1-((R)-1-(2- (difluoromethoxy)pyridin- 4-yl)ethyl)-3-((1R,2S)-2- phenylcyclopropyl)urea | 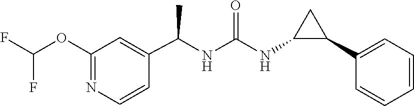 [SMILES: C[C@@H](NC(=O)N[C@@H]1C[C@H]1c1ccccc1)c1ccnc(OC(F)F)c1] |
| 39 | 1-[(1R,2S)-2- phenylcyclopropyl]-3-[2- [3- (trifluoromethyl)pyrazol-1- yl]pyridin-4- yl]methyl]urea | 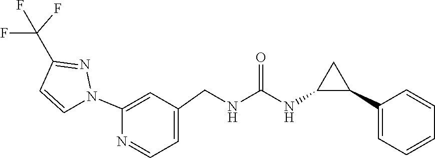 [SMILES: O=C(NCc1ccnc(-n2ccc(C(F)(F)F)n2)c1)N[C@@H]1C[C@H]1c1ccccc1] |
| 40 | 1-[rac-(1R,2S)-2- cyclohexylcyclopropyl]-3- [[2-(1,2-thiazol-4- yl)pyridin-4- yl]methyl]urea | 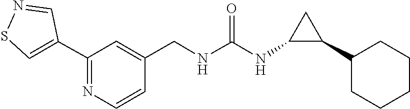 [SMILES: O=C(NCc1ccnc(-c2cnsc2)c1)N[C@@H]1C[C@H]1C1CCCCC1] |
| 41 | 1-[[2- (difluoromethoxy)pyridin- 4-yl]methyl]-3-[(1R,2R)-2- (trifluoromethyl)cyclopropyl] urea | 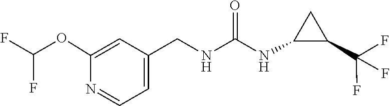 [SMILES: O=C(NCc1ccnc(OC(F)F)c1)N[C@@H]1C[C@H]1C(F)(F)F] |
| 42 | 1-[[2- (difluoromethoxy)pyridin- 4-yl]methyl]-3-[rac- (1R,3R)-2,2-difluoro-3- methylcyclopropyl]urea | 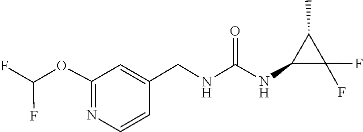 [SMILES: C[C@H]1[C@H](NC(=O)NCc2ccnc(OC(F)F)c2)C1(F)F] |
| 43 | 1-[[2- (difluoromethoxy)pyridin- 4-yl]methyl]-3-[rac- (1R,2R)-2-(oxolan-2- yl)cyclopropyl]urea | 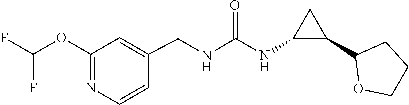 [SMILES: O=C(NCc1ccnc(OC(F)F)c1)N[C@@H]1C[C@H]1C1CCCO1] |
| 44 | 1-[2- (difluoromethoxy)pyridin- 4-yl]methyl]-3-[rac- (1R,2R)-2-(1,1- difluoroethyl)cyclopropyl] urea | 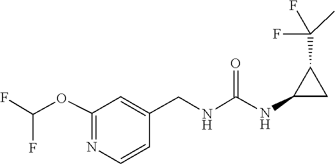 [SMILES: CC(F)(F)[C@@H]1C[C@H]1NC(=O)NCc1ccnc(OC(F)F)c1] |
| 45 | 1-(2- cyclohexylcyclopropyl)-3- [2-(oxan-4-yloxy)pyridin- 4-yl]methyl]urea | 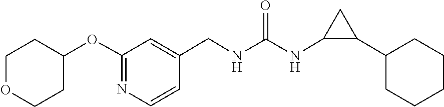 [SMILES: O=C(NCc1ccnc(OC2CCOCC2)c1)NC1CC1C1CCCCC1] |
| 46 | 1-[[2- (difluoromethoxy)pyridin- 4-yl]methyl]-3-[rac- (1R,2R)-2- methylcyclopropyl]urea | 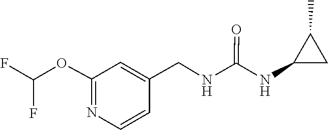 [SMILES: C[C@@H]1C[C@H]1NC(=O)NCc1ccnc(OC(F)F)c1] |
| 47 | 1-[[2- (difluoromethoxy)pyridin- 4-yl]methyl]-3-[rac- (1R,2R)-2-methyl-2- (trifluoromethyl)cyclopropyl] urea | 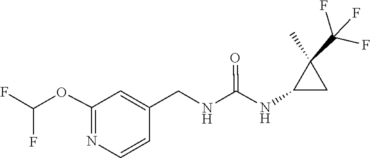 [SMILES: C[C@]1(C(F)(F)F)C[C@@H]1NC(=O)NCc1ccnc(OC(F)F)c1] |
| 48 | 1-[[2-(2,3- difluoropropoxy)pyridin-4- yl]methyl]-3-[(1R,2S)-2- phenylcyclopropyl]urea | 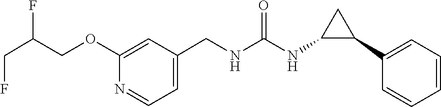 [SMILES: O=C(NCc1ccnc(OCC(F)CF)c1)N[C@@H]1C[C@H]1c1ccccc1] |
| 49 | 1-[[2-n- (difluoromethoxy)pyrimidin- 4-yl]methyl]-3-[(1R,2R)- 2- (trifluoromethyl)cyclopropyl] urea | 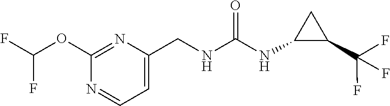 [SMILES: O=C(NCc1ccnc(OC(F)F)n1)N[C@@H]1C[C@H]1C(F)(F)F] |
| 50 | 1-[dideuterio-[2- (difluoromethoxy)pyridin- 4-yl]methyl]-3-[rac- (1R,2R)-2- (trifluoromethyl)cyclopropyl] urea | 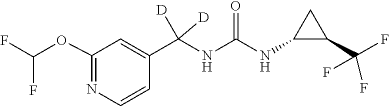 [SMILES: [2H]C([2H])(NC(=O)N[C@@H]1C[C@H]1C(F)(F)F)c1ccnc(OC(F)F)c1] |
| 51 | 1-[rac-(1R,2R)-2- (trifluoromethyl)cyclopropyl]- 3-[[6-(2,2,2- trifluoroethoxy)pyrimidin- 4-yl]methyl]urea | 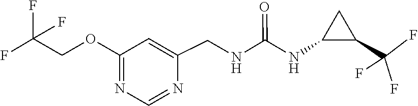 [SMILES: O=C(NCc1cc(OCC(F)(F)F)ncn1)N[C@@H]1C[C@H]1C(F)(F)F] |
| 52 | 1-[[6- (difluoromethoxy)pyrimidin- 4-yl]methyl]-3-[(1S,2S)- 2- (trifluoromethyl)cyclopropyl] urea | 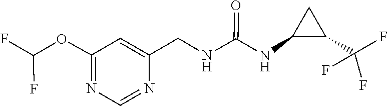 [SMILES: O=C(NCc1cc(OC(F)F)ncn1)N[C@H]1C[C@@H]1C(F)(F)F] |
| 53 | 1-[[2-[(2R)-1,1,1-trifluoro-3- methoxypropan-2- ylJoxypyridin-4- yl]methyl]-3-[rac-(1R,2R)-2- (trifluoromethyl)cyclopropyl]urea | 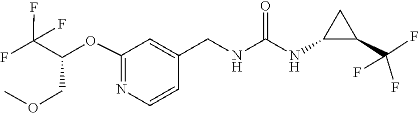 [SMILES: COC[C@@H](Oc1cc(CNC(=O)N[C@@H]2C[C@H]2C(F)(F)F)ccn1)C(F)(F)F] |

28. A compound of formula (I) or (I′), or a solvate or a pharmaceutically acceptable salt thereof, according to any one of claims 1-26, wherein such compounds of formula (I) or (I′) show Kv7.2 EC₅₀ values and/or Kv7.5_7.3/Kv7.2 selectivity ratios in the range of: i) EC₅₀<3 μM, Select.>10×, or (ii) EC₅₀<1 μM, Select.>30×.

29. A pharmaceutical composition comprising a compound of formula (I) or (I′), or a solvate or a pharmaceutically acceptable salt thereof according to any one of claims 1-28, for use in the therapeutic and/or prophylactic treatment of a disorder, disease, or disability associated with Kv7.2 in a subject in need thereof.

30. A compound of formula (I) or (I′), or a solvate or a pharmaceutically acceptable salt thereof according to any one of claims 1-27; or a pharmaceutical composition according to claim 26; for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

31. The compound or pharmaceutical composition for use according to claim 30, wherein the disorder, disease, or disability is a behavioral disorder which is Attention Deficit Hyperactivity Disorder (ADHD).

32. The compound or pharmaceutical composition for use according to claim 30, wherein the disorder, disease, or disability is a mood disorder which is depression.

33. The compound or pharmaceutical composition for use according to claim 30, wherein the disorder, disease, or disability is a neurodevelopment disorder selected from autism spectrum disorder (ASD) and syndromic developmental disorders.

34. The compound or pharmaceutical composition for use according to claim 30, wherein the disorder, disease, or disability is a syndromic developmental disorder selected from Dup15q syndrome (Dup15q), Fragile X syndrome (FXS) and Angelman syndrome.

35. The compound or pharmaceutical composition for use according to claim 30, wherein the disorder, disease, or disability is an epilepsy selected from broad pediatric epilepsy, West syndrome, Ohtahara syndrome, and epileptic encephalopathy.

36. The compound or pharmaceutical composition for use according to claim 30, wherein disorder, disease, or disability is a neurodegenerative disease selected from Alzheimer's disease, and motor neuron diseases.

37. The compound or pharmaceutical composition for use according to any one of claims 30-36, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

38. A compound according to any one of claims 1-28, or a pharmaceutical composition according to claim 29 for use in therapy.

39. A compound according to any one of claims 1-28, or a pharmaceutical composition according to claim 28, or a pharmaceutical composition for use according to any one of claims 30-36, for the manufacture of a medicament for use in the therapeutic and/or prophylactic treatment of a disorder, disease, or disability associated with Kv7.2.

40. A method for the therapeutic and/or prophylactic treatment of a disorder, disease, or disability associated with Kv7.2, which method comprises administering a therapeutically effective amount of a compound according to any one of claims 1-28 or a pharmaceutical composition according to claim 29, or a pharmaceutical composition for use according to any one of claims 30-36.

41. A kit for use in the therapeutic and/or prophylactic treatment of a disorder, disease, or disability associated with Kv7.2 comprising:
a) a compound according to any one of claims 1-28, or a pharmaceutical composition according to claim 29, or a pharmaceutical composition for use according to any one of claims 30-36; and
b) instructions for use.

42. The invention as hereinbefore described.
